SEQUENCE LISTING (13 sequences)

SEQ ID NO: 1 - protein, 188 aa, human
MTEYKLVVVGACGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 2 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 3 - protein, 189 aa, human
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 4 - DNA, 45675 bp, Homo Sapiens
ggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggtacggagcggaccacccctcctgggcccctgcccgggtcccgaccctctttgccggcgccgggcggggccggcggcgagtgaatgaattaggggtccccggaggggcgggtggggggcgcgggcgcggggtcggggcgggctgggtgagaggggtctgcaggggggaggcgcgcggacgcggcggcgcggggagtgaggaatgggcggtgcggggctgaggagggtgaggctggaggcggtcgccgctggtgctgcttcctggacggggaaccccttccttcctcctccccgagagccgcggctggaggcttctggggagaaactcgggccgggccggctgcccctcggagcggtggggtgcggtggaggttactcccgcggcgccccggcctcccctccccctctccccgctcccgcacctcttgcctccctttccagcactcggctgcctcggtccagccttccctgctgcatttggcatctctaggacgaaggtataaacttctccctcgagcgcaggctggacggatagtggtccttttccgtgtgtaggggatgtgtgagtaagaggggaggtcacgttttggaagagcataggaaagtgcttagagaccactgtttgaggttattgtgtttggaaaaaaatgcatctgcctccgagttcctgaatgctcccctcccccatgtatgggctgtgacattgctgtggccacaaaggaggaggtggaggtagagatggtggaagaacaggtggccaacaccctacacgtagagcctgtgacctacagtgaaaaggaaaaagttaatcccagatggtctgttttgcttggtcaagttaaacccgaagaaaacccgcagagcagaagcaaggctttttccttgctagttgagtgtagacagcaatagcaaaaatagtacttgaagtttaatttacctgttcttgtcctttcccctatttcttatgtattaccctcatcccctcgtctcttttatactaccctcattttgcagatgtgttctacatctcaagagttattacagtactccaaaacagcacttacatgattttttaaacttacagaggaattgtagcaatccaccagctaaccgcctgaaatagacttaaacatgtgcatctcctttttttttttttttttgagacacagtctcgctctgttgcccaggctggagtgcaatggcgcggtatcggctcactgaaacctccgcctcctgggttcaagcaattctcctgcctcagcctcccgagtagctgggactagtaggtgcacgccaccatgcccagctaatttttgtatttttagtagagacagagtttcatcatgttggtcaggatggtctccatctgctctgttgcccaggctggagtgcagtggcgccgtctcggctcactgcaacctctgcctcctgcattcaagcaattctcctgcctcagcctcccgaataactgggattacaggtgtctgctgccatgcccggctaattttttgtatttttagtagagacgggggtttcaccatgttggtcaggctggtctagaactcctgacctcgtgatctgcccgcctcggcctcccacagtggcatgtgcatcttatagctgaagtctaagccttcttaaatcttgagatccatcaaaacagacaggttttctaattgttatacaatgtatatgttatgtttataatagaaatcattttacaaataagttataaatgggaaaggtctatttgtaattatcagctcagaattaaccataaaactggtgtcactgaagtgactgaggtccaaaatgctgactctgcatgttatagactacagatatcaaatatggttgctaacaatagtttactttgagactgtagccatccacagtatatttgcttttaagagatggtagatggtaattcagttttatgaaaaataaaaatgaattttcttccattacaaaattgttggattcgagtccagtccactccttactagcttttctaactctcggtgagggatcccctcccagcccatgatcttcatttggtaagactcctttggaacccagttctctctagtggatttaaatgtgatttggttttaaaaatctcattcaaggaattttttttttttctggaaacaaccaccgcataaacaagtaaaccggaagatacatgtggctctgaattcatatatatacacaaactctaatccaatgtctgtccacagtatttcctaggctagtaaactttttggccttaacgacccctctaccctctttgtttttttgagagagagagtctcactctgtcacccaggccggaatgcagtggcgcgatctcggcccgctactacctccgactctcaggctcaagcgattctcccgcctcagcttcccgagtagccgggattacaggctcccgccaccgggctaattgtatttttagatacgggatttcaccatgttggccaggctggtctcgacctcctgacctcaggtgatccgcccgcctaagcctcccaaagtgctgggattacaggccaccacacccggcctacactcttaaaaattatcgaaggggccgggcacattggctcttatctgtaatcccagcactttgggagactgaggcgggaggatcgcttgaggccaggagttggagaccagcgtactcaacatagtgagaccttgttataaagaaaaaaaaaatccaggattaaaaaaaatctttgatttgtttgggatttattaatatttaccgtattggaaattaaaacaattttttaaaatgtattcatttaaaaataataagcccattacttggtaacatgaataaaatattttatgaaaaataactattttccaaaacaaaaccaaaacttagaaaagtggtattgtttcacacttcagtaaatctctttaatgatgtggcttaatagaagatatggattcttatatctgcatctgcattcaatctattatgatcacacatctggaaaacttgtgaaagaatgggagttaaaagggtaaaggacatcttaatgttattatgaaaacagttttgacctcttgcacaccagaaaagtcttagtaacctgaggggttcctagaccacattttgagaactgttttaggctatgcaaactggttggggggaggttggggtaggcagagagctagaagatacattttagtgtaattctcctcatctattcctaattgctttggcctacatttgaaataaagcgtggaggcaaacgggataagatacatgtttgtagtggttgttaacttcaccctagacaagcagccaataagtctaggtagagcagagtaaggcggggaactatgccgtgaccgtgtgtgatacaatttttctagcctgtggtgctttttgcggcagggcttaggagtaaggttagtatgttatcatttgggaaaccaaattattattttgggtcttcagtcaattatgatgctgtgtatatttagtgtttatctacaatatatgcacattcattaatttggagctactcatcctataataaatagttgtgcatttactcccatttttttctgcatttctctccttatttataattatgtgttacatgagggaaaggaggtgaaattaaacattcatattatttcaaaaaatttgaaacaactaactaaaaaatatgttttattttctgtatggtgtttgttatacaatctgtcaatattcatgcacctcttgggagacagtgtatgaaaagcaaagagtaacagtcacatggattactgattactgagatatattcacttgcatctttttttttttttgagacggagtggctctgtcgcccaggctggagtgcagtggcgtgatctcggctcactgcaagctccgcctcctgggttcacgccattcttctgcctcagcctcccaagtagctgggactacaggcgcccgccaccacgcccggctaatttttttatatttttagtagagacggggtttcaccgggttagccaggatggtcttgatctcctgacctcgtgatccaccctcctcggcctcccaaagtgctaggattataggcgtgagccaccgtgcccggctcacttgcatctcttaacagctgttttcttactaaaaacagtgtttatctctaatctttttgtttgtttgtttgttttgagatggagtcttactccgtcacccaatctggagtgcagtggcgtgatctgggctcactgcaacctctgcctcccgggttcaagtgattctccttcctcagcctccccagtagctaggactacaggagagcgccaccacgcctgattaatttttgtatttttagtagagagagggtttcaccatattggccaggctggtcttgaactcctggcctcaggtgatccacccgccttggcctctgaaagtgctgggattacaggcatgagccgccgcacccggctttctaatctttatctttttttgtgcagcggtgatacaggattatgtattgtactgaacagttaattcggagttctcttggtttttagctttattttccccagagatttttttttttttttttttttttgagacggagtcttgctctatcgccaggctggagtgcagtggcgccatctcggctcattgcaacctcggactcctattttccccagagatatttcacacattaaaatgtcgtcaaatattgttcttctttgcctcagtgtttaaatttttatttccccatgacacaatccagctttatttgacactcattctctcaactctcatctgattcttactgttaatatttatccaagagaactactgccatgatgctttaaaagtttttctgtagctgttgcatattgacttctaacacttagaggtgggggtccactaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttattacaagataattatgctgaaagttaagttatctgaaatgtaccttgggtttcaagttatatgtaaccattaatatgggaactttactttccttgggagtatgtcagggtccatgatgttcactctctgtgcattttgattggaagtgtatttcagagtttcgtgagagggtagaaatttgtatcctatctggacctaaaagacaatctttttattgtaacttttatttttatgggtttcttggtattgtgacatcatatgtaaaggttagatttaattgtactagtgaaatataattgtttgatggttgatttttttaaacttcatcagcagtattttcctatcttcttctcaacattagagaacctacaactaccggataaattttacaaaatgaattatttgcctaaggtgtggtttatataaaggtactattaccaactttacctttgctttgttgtcatttttaaatttactcaaggaaatactaggatttaaaaaaaaattccttgagtaaatttaaattgttatcatgtttttgaggattattttcagatttttttagtttaatgaaaatttaccaaagtaaagaccagcagcagaatgataagtaaagacctgtaagacaccttgaaggtcatggagtagaacttccatcccaagcagatgaggatttatttaatctcaaagacctccaggaggggacattccccaactgtccttgttaactcattttcagaacatatttattagcatattttacatgtaatttggatcttcatgttaaatttaacatcagtggagatggaaaataagcatatcgccttgtctttgaaatagccctatattgttagattgtttcttaggcttctttaccctgggttaagcagtcctaatactttagcatttattctacatctagtgtactaatttaaaaaaatcagttctgaaaaatttctaagaactttcttcaagttccaagctgtgaaatctagaacaggtcaaagtgccttattaacgtactgtactgtgtagtgtcttgaagagacactttgcgctgaggcaagttctgagggcattgggtggccttgggaagatatttatgcagtttagaacctggagaattgattagataactaatcataaggaaacgtcacatatttttggtactataaaaaagtggagaaataatgcctatttgcaaagatttgatttaaacatagaaacaactttatttggcttccaattttaagaatttacagcagtaaaggggaacagtctaattgaagtagactgcctatgcaatagtctctgtatatttacttttgacaagttaattcaatgtgtactatagttttgtttctttgaagaggtttgaatagtgcacccattttaatctgtattgcaaattcagggttacttggcagactctactatttaaatcagatgtaaaaggaagttttaatataattcactttatgcctgaaagttttcctgggattttggaaggtgattttactggaaatgctgtctgtcttccctgaaaatctgagaaattccattacactttgtttccaatcagaggtcatgagtgctatatgagtatatacagcatgacgtcatgaatgtgataaagtgggttaggaaaccttttgctaatgattgttaaaatgcaatataaatgttgaagaaataaagctaacagttaagcctttatttgggcggaaggctgaaaaagtttataaacttaaacctataactctgcttatgatttctgccaaaccagaagacttgactctgggaagcattggttacctgtgaactttgaaactgacggtccctgacgtagtttagtcacctgggaaaaggtatctgagattatctcttatctcccaagttacagtgagtctctgagggaactgacacattacattaagttcttggtgtagttaaactgtaagaaaggcaggagaacttagtagttaaatagttggttaaatggaaatgctgactccatgttattgtaaaaagttaaaaatttaggaggatatggggatttcactgccattgcaggttttgattggtatttaccaatccgtgtgggtcagagagaaaattagaaaggatatgactgcacattttggaattattagcagtttttctacatttaaaatggaaataaattttttaaaaatttaaatcaagtaatactgtattttttggtgatttagatttttcaaaatttacactaagagatagtaaggagggtggctattgtttctttcaataatgtctctgagaggttgtaactcatctaaggatacgtagctaataagtggtaggatttcaatttaaattctctgagaccaagttaagtagaatttgcactgtactcttgtataactttttaaaactgaaaattagctatctttcaaattaagaaaatatttactaatggagactaattcagatttgtaagtataccaaaatttgaacttagcctgctatctaatggcaacttagtggcagaggtatgatgtaaaatcattcaggtatgacacatagatggagtatgtttgtattcgaggctgtgcacataatcacctttacttgtattgtgaagtatatattgttatcttttatgaagcccactaaagagataatgaaatacctcgttattagggcaagattattgaaaactcaaaatagcccccaaacacaatacttggctagaaatatatacctttatagttcagagatcatttattatcaaaaccctgaagttttttttctaaggtaaaatttggtggaagaggaaaagtctcgttttaaaaaaatgtaggtagttacagagatcagaatgattagttgatcacttaccaaatatatattaagtatctactgtatataatatgctagtaagaataaatatagcaggaagtattttttcccaggctctaattgtttgacatcagcatgcttttattgtggcacttataattcagttcaagtattatgcccctctttgatggaacagtttcctattcagtaaggaagaccagattaatcattggattggtttgtttcatctttagtgttctgagctgtagagtatttatttaccaaggtttattttaatttttattttatttttatttttccatgttcattgtagaattcattttacctacgaatgaagtatgtagattatagagagaaaatttgtaaaattaaactgatactgaagactggtataagaaaagccttatgtaatttgtaagctgctattcttctgagtttatacatatatctttagtaatcaatgagggatggttgggtgactgccctccaggggacatttggcaacatctggagatgtttttggttgccacaacttggggagagagtactgctactggcatctattgagtagatgctattactttaaatggcaaagctgcagttacctttgcaccaacctaatattaaacttcctgcagtgcacgggaaagcccccacaacagggttatctgaccccaaacctcaatggtgttaagatccaaaccttgatatgttaacctgtagctttaaacatcctttaaattgtcaaattcatgtccctgacataaggtttatgttagattttcaagtataacaaagatttaaactttaacttttgtacgttaatgatatgttagcttactccagtcttctattaaaacattctgtttttaaaatcagagacacacagcaattttataaatcatttctcttcaaggctgtgaagctctccccacttttgtgagtgccctctactggtcaaattatttgctttataacaagtaacagtgaaatcctaagtttgtgtagtttcgctgtttaaattatgggtggcatcaatttataaatatattcgttttatttaaaagtcttatatgattgatttcgtatcatttttgctctctgctaatattaatataaagattactgtctgtattagttaggcctaactaagtaggtgagtatagtgaactaagaaaggaaacgaggcagtatataagaaaatagggtggttcagttgttaacacttactgagcttactttgttgaagggactaaaaggcagcagtgtggctctctgagcttctttgcatgcactcaggagctgcttaatggagtccaaggcttggtggtgtgttacaggggatgataggagggtcctattcagaagtggcaaattgtgaaagtgcacattttgtagagttttataggactgtagaatagttgtgagcacctgatttttagaataaacagaaaactcaggtactgtatttaggtcaaattaagaataagtatttattaagacctgaatataaaactttactggtcatggtttttttctaccttgggtttttataaatccaaagatttaaaaacatacaaatggaagttggtaatggaattaagtgaaaggaaaaaatgattttatggtttggaatctcctaagattctggttttaacaatacaactaattccttaatcctagaaatgttcttcactgcccactttgtaccatgcagtcttcctgtgggctagagatacactgaggcgcaaaacagaccagattcctgccttcatggagcttattagttttaggtatctctagatttcttgtaatacctattacaatgcctgcacatcagttcattcatgtgggttcaacgtagtactcagtacatggcaaattcaagttttacttttcggaacttcatggatttttttcctcagaatatcttttatccataattggttgaatctgtagatgcagtacccatggatatggatggcccactttattttgaagagcagtgtttctaggcaatcatgctaattatatatgacttaatttagaggctttatacttaagagcattacatttctggcgtctcttaaccattattatttcataatgtgtaggttatggaacagttaaattattgggatcttaatatagaaattagtagaaataagccagatatggtggctcatgcctgtaatcttagcactttgggaggctgaggctattcgctgtactattttttactacttttctataggtttgaaattttttcaaaataaaacattgaaaaaagtaaggtaggtagtgtgtccctccttaatcctttcaaatattttattttcactatttctattaatttttttttttgtttttgagatggagtctcgctctgttgcccaggctggagtgcagtggcgcgatcttggctcactgcagcctccacctcctgggttccagccattctcctgcctcagcctcctgggtagctggtattacaggcatgcaccaccacacccaattactttttgtatttttagtagagacggggtttcaccatgttggccaggctagtctcgaactcctgacctcgtgatctgcccgcctcagcagtgtcactgcttctagaccgttttcaaggcacagagcttagaaatgcatgttactaagaaatcaagagttaactatttttcaccttctttctcccgcagtgagaaccctggttctaccctgtttctccttgtgtaaattttaatgctaaactatacacttgtgaaataaaaatgataatgtcattcttaaattatggatcttgcagtgttatctaagtaacatagattgagtgatttaactttaggtttccttatttgtggaatttggataaatatttttcacccttgagaaaagtgagactcctttctcatcatcagagtatccttaaaccattaaggcaaacatttgggaaaaaactgagctatctggctgcataaaaattaagttttctttaacaaagatagaagacaaatgaaaacctagaaaaaccatttggttcaagtaacaggaagctatcttatatatgaattagagaaaagcaaacacacaaatagaaaaaaagggatggggggtactaaagatataaatagcttgtctaccaaaaaagaaataaaataaataacatgaacatataaaaagacacttacttcatgaatgtgatgcaagttcaaacaataaataacatttctgtactttcatattggctaaggttaaaatgataactgctaggaagggtatggagaagtgtgcgccttgcactgtagtgggagtatagaccctcagactttatggaggtcagtctggaaatatgtttcaaaatgtaaactacatgtcctttgaccaggtaattcaacttcttgaaatttatccaaggatttaattggataaatgtttaagatgtatatataagaatgtttactgcagtgttgtttatgattttaaaaaaatggaaatcatcttcatgtctaccaatagagaatgggtgaataaattatggtatgtccatatatacaaattacatagttgttggaaatattaggtagatttagatatactgatgttcaaaaatgtccattatgtaagtgaagctgggtcacagcaccttgtgttgagtatgatttcatctagaaacaaaattactccctcatcctttgttgtgttttagttttttaaaataagcttataccattgggctgggggaaaagtaaatactcgttttggagagagaaaagggcactaaagtttcagataccgttagattatttcatgcttatttttcaagcctcaataaattacataattcacatgtagtcttggattaaggaaattgctattaaggctaaataaataatatgagaggtatataatataaaatatgaacattatattggcattaagattggatccacggtcattccagcctctcattcttacctggacttcaagtgatcacttgtgggcaaatgccatctgacttgaacaggttacacatgtatgctcattatatcgttattttcaaaatttgtcatataaattttccttgagttcattcagatttttgaactagttttttctcttgggagtagtacacacttaattctctctagtactaagctaatgttcaccattcttataattttaagtatccagcatttagtaaagaagtctttgttttctttatccttacttttagtgaatgtcttagtttttaattgaaaattctgccatgaaaataagctctttaacatcttcactccctaatcaaaacagaaatccttcatagccttcagttgtagctatccttccctgtgatttgtccagctccattatatttattttgaaatatggtgaccagttttgcaaaattatttcaactgtaggtgcccagtgattttgtaaggagaagatactgtttctgaacagttctcagtagccagtggcctgcccctactttttggcctgcgtgtagtatataaaataatgcagttaactttttatagcacttttcattttataaagagattttcatggtctttaatattaatctatgtataaagtcctgtatgcagttttacctactttcacagctgaaggaacaatagcttagagaagatgtgagataaagtagtttgcccaagcccatagcacaaataagtgaagttcttcggctgtccatggatcgaagactcccaagtctatctctagcctggacttctgtcctgagcaccagacatgtatgtatatcaagatgcctgcaggtcatatccaccaggacaacccatgagtacagggaattcaacatgcccaatatcactcatcttttccttcgccctcccctttgtactcatcccctgtcggtaagctctgttattttaaaaaattgaaatgtattcacatagcatacaatttacacttttcaagtgtacatggtttttagtatattcacaagggttgtgcagtcattactactaattccagaatgttattatcaccccaaaagtcccacatccattagcagccactccccaatcccttctcccaccagcctctaaaaactgctaatttttccatctctgtggatttgtccactctgattatttcatataaagagaatcgtacagacgtggccttttgtgtctggcatcctccacacaggatgatattttcagagttcgtctatgtttttgcttgttgatcattccttcattcctttttctggctgaataatactctgttatatggatataccttattttgtttatctgttcatttgatgggcatttgagtgatttcctctttttggcaattttgaataatgccactataaacatttatgtacacgtttttgtgtgaccatatgttttcacttctctcgggtgtatatctaaggtacagttgctgggttatatggtagctctgtctttgactttttgaggaactgccaagtggttttggtagtgattgtactgtttacattcctaccaacaattttacctaagtatttctcaaatctatttaatcttttcggtccatactgctgttgctgccttagttcagattttgtcatttcttgtaataattcgtagctcatctcccagtctctgctcccctctctccctccctcccccttcttctctctcttatttccacccatttttaacatttatagaagtcaaaagtctagttcagaaagcagaaaccatactagatatttcagcacagagaactaattaggtgttggaagactgaaaggcaaaaaaacactgaagtaacacagtaacatcaagaatgggcactactcctaagattcagggaatgctgggaagatttggggtttatcagaactggaagctcagaggaggggccccttgtcgctgaggcttaatccctgcagaggtgcctttggctgctactggtgaatctgagtgggtatgatgagtcagtgtctgggaagggccaaaacattttgtccctttctataatttgtcatgataatgctagtaatgaatctgatctcccttcctattttaaaaaccttttagtgattttgtataggatgaagtttaaaactccttacttaatatacacatgaccctccgtaagctggcccctgcttgattgtccagtttcacttcttggtgcttattctaaggcctctaagccttagagatcctctaagcctttgagatccccaaaccctggactgcggactggtacccacctgtgtggcctgtgaggaactgggctgcacagccggaaggaggtgagcattacttgccttagctcctgtcagatcggcagcattagattctaataggagcgtgaaccgtgttgtgaactgcccatgcaaggatctaggttgcatactccttaggagaatctaactaatgcttgatggtctgaggtgaaacagtttcatcctgaaatcacccccaactcggtccttggaaaaattgtcttccacgaaactggtccctgatgccggaaaagttggggaccgctgttctaagctaaagttatatggagctccttggttctgtgtcctcaacatgctgttctatgttttttacattctgtttgctccttcctgcttggaatgtccttcccctccccgtctttcttaatgcatacaaagttgatctctcctgtgtgccaccattgtacttcgtcttgcatatggtgttacattcattttattttaattatttatttacgttcatgtctcttccactcaccttagttgcttgaggtcagaaactatataatgtgtgacacggaatgtgacacctagattttcaataagtgtttctatgatacaagggagactgatgtgggtagatgggaatgaactcatcaacctctgtttacataccctaaattccctgtttcttccctattataattctgacagtctacaaccgtctttgatggcttataaacggaaagtgcggaacacatcattctacagtgaatttaaataacctttcggaagagtaacgtaaagtacttgagcattaattgagtaaaagtttctcatcttttcctacaggtgttattaagcagtatgtaaaaagtccttacaatacttaatacattaagaaaacatacaatttcaagaggaaatccccgagtaatacattattgacattttcagcagttctagttatattgagaagagcatctcatggaattggcagaatgaagatggagattaaatgagatgatgtttgtaatatgcttatgacagtatctggcatataagtaagggctcagtaaatgttgactgctgtaattactattaatagtaatatgattacctttagtaaaagttattagtttctttaggttttttgtttactacaatatagtaaacaaaatctatacttggaatgtatatattgttttgttttgatacatggaatatgtctctgtgtcagagtcactgcctgagttggaaaacccatactcgagtatgttaaaaggtgaacacactgaataatttagttattaattataatggaaaaatgacaaacttgatgttctggttaatgaggttatcttatcttgaatgagttagcttttaaattcctcaaaataaaggcatttaataaaccaggaaacacttcattaaaaaaattatgcaagtcagtgtaaaagaagattaaaattccacatgggcaaaggacacacgttggcgataaatatgcagataagaaaaaaaacctatataacattattactcctcaaagaaattggtatgaaaacaataaaaatgtgtagcttatcaaaccaacaaaaatttaaaaatatgaaatccattttaagtaatgataaaatgggtgcactcttagtgctttatagaatagtagtataatgaacctcatgtgtgtaccaaccagctctttcatatcttaacatttagcaacatttgatttagctctttcttttttccaagatagaaaagttaatattgttgaagactcctgcattcttttccctagtcttattttcttccctcccataaatgtgttaaaatctctgtgtgtattgttttggttgtatttttacataaaactttacatattatataaaatttaattgaaggtaaaatttattaaattattcttaatatatattgtaatttaaaaattaacagcttcattgtcttgataaaatttatggtatcttaaacatgtgcttgtttttctaagagaacattgaaacatagattttaaaacaaattgttgaaagattaaaaaatctgcctttgcacactgttacattgaaagtggggcatttgtcgtgaacattcatttcaaatatgtagtatcttcagaatatttgagaaggatttgtattatataattgaaaaatctgttaaattgtatttatgttaactgcttaattctaataaaatttccattcattttttagtatctgcatatatttacatcaaatggattcattcacttatttaagaggcagtactaattacctatagcgttcaagactgttaggtagagggtgtgtagtggtgagtacaacaggcgtgagccctaccaacacggagtttaaagcctagtagaggatatagacttaaacaatttcacaagtaaatacataattacaaattataatacatgctatgaaggaaacataggaggtaccagagaaggaagagtgctttgcatttttatttttaagaccgaagagtgctattggaggactttgagcaagtgaatgacatgatctaacctaccttcgttcattcattcattcattcattttcttccttcctggctcaagcagtcctcccacctgagctccccaaatagctgggactacaggtacacactaccacacctaatttttttttgtattttttgtatttttgatgggattttaccatgttggccaggctggtcttgaactcttgacctcaggtgatccacctgtctcggcctcccaaggtgttgggattataggtgcctagcccatggtgcctagccctaacctacatttataaactatcacttgctgctgtgtggagactatattgtgagattaacagcagggatacctgctaggaagcaattgctgcagattgcctgagacaaaatagttatcatggactagggggatggtggtggtggtggtggtaggtggttggatgtaggatatattttgaagataggtaaatggtgcaagattatgggtcagttttaaatgcttaagtaaattttctttgtaagacattttaggatgccatgttaagaatctctttataactgtcatttaaaaaaaaaccacatattttcttagcataatttcccatagtaacattactatgtcaaaggctatgaacatttgaatgactttagataaatactgtaattgctttccaaaaatattgtgcttattatgtcaccagaaatgtttgaattctgtctacaattcagtcttgccagtatagtacatttcatttagaaaaattttttactatgtagatggaaaaaataatattttagctgggagtggggggactatggggaataactttccttcatttaatattttattgtgagttagtttaagttactttattttatcgtagtttcctaaggctacaaattagtaaccttggtaacttatgtacctaatttaaaagtttacttttttgaaaggctggaaatactaattaaaaacgtaacaccttcatccttgtctttgctccattattaactagtttcattacagaatctctgtgttttaaaatcagatgggttttcataaccagtactttctcagagtggtaaatttaaaaaaatatataaagagaataaataatatttgttgagaatacttcaaataatgtgaagagttattaacttacagcaggagttggcaaacttttctataaagggccatatgggtctttgtcacaaagtcttgggtttttgtttttgtttttttaaacagctatttaactattcctagctaatgggcaatacaaaaacagtgggcaagatttggcctgtgggcagtagcttgctgaaaccttatttagactctaaattttttgaaagagtctacattgatgcatatttttttttcttcctccaaatacagttgacccttgaacaacatgcgtttgagtgaccatgggtccacttgtgatacacgtttttttcccaaccaaatgcagatatggagggctgacttttcatatacctggatgttcctgggccaactgtaggactagaggctgggggggtcttggaaccaatgccgtgtgtataccagggatgactgtttcttatggcctgacctgaagttggaacagaatctttattaatatataatttttgttgcgtttgttttctctttatatttatccattctttttagatcgtatttcatttaacactttttcttctttagtttttaccaagttgcactgaaaatagctcagtgactaattgcacttctaagagtgaggaccctagttaaaattaactctaaaaatactgaatttttaacctaaaccttttatttctaatcaacagtattatttatgagtaggttatagattactttgaaacggaatgtgtctcagaactttgctatcgatatttttaaggtctggtagggaaaagataataggaatgagatttatcagtgaataggggactgctttcccagtttctcggtcgcactggtgtattcaccatggaagcatcttatgaaatatgtacataaactactaatatcccacattacaggttgactattctttatctgaaatgcttaggacctagaagtatttttggattttggtttttcagagtagggatactcagcctacattggtaagtaaagaatgtgaggtgacaggctgggcgcgatggttgacgcctgtaatcccagcactttgggaggccgaggcggatcacctgaggtcaggagttgaagaccagcctggccaatctgtactaaaaatacaaaaattagctggacacagtggcacgtgccagtagtcccagctactcaggaggctgaggtaggagaatcgcttgaacctgggaggcggaggttgcagtgactcgagatcgtgtcactgccctccagcctaggcaacagagcaagactccatctcaaaaaaaaaaaaaaaaaaaaaaaaaaagaatgtgaggtggcagcaataggtaggaagagtctttggtcagctttacatgctctgtagccatgcctgggtaatgggttgactctaagactctgtgctttgctcccacctcctgctttttcattactctttagaatggtttttaatttgtgatctataggagttctttcaagtatttaataagagaataggctaaattaagtaaatgtcaactgaatgctcaaatctctactaaagagcctcttatttagaaaataaatatccatcttttttttctgactggtgagataattaatttttattacagatggtttggaaaataccatatgctttaaaagataagcacaaaattatagtctaatatgtaggttttcatactttaaaaaattgaaaaccaaagaaaaacatttaacatagcatctagtacaaagaaaagagataagcaagagataaatgtcttttttgggacagagttttgctgttgttgcccaggctggagtgcaatggcacaatctcagctcaccgtaacctccacctcccgggttcaagtgattctcctgcctcagcctcccgagtagctgggattacagtcatgcaccaccaggcccaggtaattttgtatgtttagtagagatggggtttctccgtgttggtcaggctgatctcaaactcccgacctcaggtgatctgcccaccttggcctcccaaagtgctgggattacagacatgagccatcgcacccggccaagataaatgtcttttaaattatctccattaaagacataacctttataacattttgatgtatatattaccagtttttaaacacatagtagatttgtataaatacataaacacatattattgtgatcatgctgcacttagacatctttatattctccttatactgtaaacattttgaaatactttactaacaacatttgtaatgaccattctttctctctttctccctctgatagaatggtctacagagtaattcataaactaaacatactttagaggctgggcgcagtggctcatgcctgtaatcccagcactttgagaggctgaggcgtgcagatcacgaggtcaggagttagagaccagcctgactaacatggtgaaaccccatctctactaaaaaaacagtacaaaaattagccgggcgtggtggcgtgcacctagaatcccagctactcaagaggctgaggcaggagaatcactcgagcccaggaggcagaggttgtagtgagccgagattgcaccacagcactccagcctgggcgacagagcgagactccatctcaaaaaaaaaaaaaaaagatacattaatactatagcctacatgtggaacattaagaaaataattgcttttatgtttatgctttatacctgttgttagccctgcttcttatttcatgatttcatggcttcacattgtaacatccctttaccatattttttgaggactgttttggcagaatgtgtgaaatcttgagcagaagtattacccaaaagtcagaagaaaatcagatttttatttcaagattctgttaaagttacccactcccttcttttacttaatcttatagttgcagttctctctctttttagaaaagaaaaaagaggcccctcaggatttgcagatgaaacaatattgctctttagagatatccatctggctgttagattatttttccacagttttcagaagtggatgaggccattagaatcttgagtattgcccatttccttatgtgtgcctttgactatagataaaatagatgcatgacaattatttataagttgattgatttttcttgtcatttaaatcatcttgaataatagagttggtagagctatcccatttttgaaattattttgttttgtcaataactttttgttaccagcatgtacacttgcattgttgactctccatataatacctttaaaaaatttttttttgtggtaaaatatgcataacataaagtttaccatggtagttttctttcatttgttttgtttttgtttttttgagacggagccttgctctgttgccaggctggagtgcagtggagcgatcttggctcactgcaacctccgcctcccgggttcaagcaattcccctgcctcagcctcctgagtagctgggactacaggcgcccgccaccacgcccggctaatattttgtattttaatagagatggggtttcaccatgttggccaggatgttcttgatctcctgacctcatgatccgcccacctcggcctcccaaagtgttgggattgcaagtgtgagccaccgcgcctagaccatggtagttaattttaagtgttcaattcagtgaccttaagtgtgttcataatgttgtgcaaccatcaccatgttgtctaaccattagcactatctgttttgagaacttttttttatcatcccaaattagaattctgtacctgtcaaatagtccccagtaatcctccctcccccagcccctggtaatctgtagtctacttttcgtctttttgaatttgcctattttaggttcctcatataagtggaattatgtggtatttgtccttttgtgttggcttacttcatttagcataatgttttcaaggttcatctgtgttgtagcatgtatatacaggttgaagcatccgttatccaaaatggttgtgaccagaagtggtttggatttcagattttttttttggattttggaatattcatagatacttaactggttcagcatccctcgtccaaaaatccaaaatcagatggagctcagtggctcatgcttgtaatcccaacacgttgggtggccaaggcaggaggatcgcttgagcccaggagttcaaccagcctgagcaacacaagaccctatctctccaaaaaaaaaaaaaaaaaaaaaaagatgaaagaaaaaaaaatccaaaatcaaatgctccagtgagcatttccttttagcatcatgtcaggctctaaaagttacaggttttggagcattttggatttcagatttttggattaacctgcattaatgctcaacctatatgaaattttattcctttttatggctgaataatgttccactgtatgtatatactacattttgtttatccattcatctgttaacagacacttaagttatttccacattttgggtattataaatagtgctgctgcgaacattggtgtacatgtatctgtttgagtccctgtttttagttattttggttatatacctaggaatggaattgctgatcatatggtaattctgtgtttaactttttgaggaactaccactgttttccacaatggcatcaccattttacattcccaccagcaatgcacaaagatttcagtgtctgtatccttgctaacacttattttccattttttgagtttttttgttttgtttttttaataatagccaatcctaatgggtatgtggtagcatctcatggttttgattttattttcctgactattgatgatgttgagcatcttttcaggtgcttagtggccatttgtccgtcatctttggagcaggaacaatgtcttttcaagtcctttgcccatttttaaattgaattttttgttgttgagttgtatataacaccttttttgaagtaaaaggtgcactgtaataatccagactgtgtttctcccttctcaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattataggtgggtttaaattgaatataataagctgacattaaggagtaattatagtttttattttttgagtctttgctaatgccatgcatataatatttaataaaaatttttaaataatgtttatgaggtaggtaatatccctgttttataaatgaagttcttgggggattagagcagtggagtaacttgctccagactgcatcggtagtggtggtgctgggattgaaacctaggcctgtttgactccacagccttctgtactcttgactattctacaaaagcaagactttaaactttttagatacatcattaaaaaagaaaaccataaaaaagaatatgaaaagatgatttgagatggtgtcactttaacagtcttaaaagcaatcgtgtgtatagcatagaattgcttggattggataaacagtggcattatatattttaaaaaataaaagttttgaaagattgaagaatttgggcattacagttctcttaaatctgacaaagctgcataaaactattaaaataatcattattatactattttatattctatttctttgagggtttagttttccaaaaactacatattaagcaaatgaatcactcagtggctatgtcatataataacgagttagcctagttataagaagtttaacattttatttaagaacattgttacagcatgtttactgtatagtctagtaatagaggaaaagacatttgggtgggtggtagtggtagtatttttatagaggagttaccaaatttcagctctattatccaagtttacccagctaatggtgttcggaaccgggaatttgagccaattctgactctgttgtctgctctgctccttcttttgtgctgtgtctttgaaagtcacctaaaattgtgagggaatgtaatttcaccccaaatttagagtttatgcacttgttatattgaaaatgattaacatgtagaagggcttttaatggaataagtggtgtagtaacttcagtgttgcctacctagaaatcaaaatctttctagttgtccactttgttttttgaaaaagtaatatgaaaattatgttaatgctttaattcaggtttttgtaaaatattttttatctttacacatttaacatacgtttctaaaattatagtctgttatatagcactttgggtctagaatttttcagtagtttctgttttactattatgatctacctgcatattaacctattaggttatagttttactatacttctaggtatttgatcttttgagagagatacaaggtttctgtttaaaaaggtaaagaaacaaaataactagtagaagaaggaaggaaaatttggtgtagtggaaactaggaattacattgttttctttcagccaaattttatgacaaaagttgtggacaggttttgaaagatatttgtgttactaatgactgtgctataacttttttttctttcccagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacaggtaagtaacactgaaataaatacagatctgttttctgcaaaatcataactgttatgtcatttaatatatcagtttttctctcaattatgctatactaggaaataaaacaatatttagtaaatgtttttgtctcttgagagggcattgcttcttaatccagtgtccatggtactgcttttggctttggtttctttctacattgaaaatttctcttcaattctgagcacatgttaacatttagaattcaagaggtggggatttttttttcccatggttacatatatatatatatatatatatatatatatatatatatatatatatataaagaacagggcaacaaatttttgcgttttctatttcggtagtacttttaaaccattatgtcatgtttctaggttaaacgttgttgtatttgaagaattttactttggcagaatttttttgaggatgtgtttatttctggagaaaggtctcattaaagaaagacaatacccagaaagccaacagaaattctgttactcatttaatgcatttttctgacaaaaattattgccagagagaacctgaattttgtttcaaaaatcatctttgttttaaaaatgactttttcttcaggtaaaataaaataatttcagttgctattatttaacctgtttgtatgaagagtttaacatataggaaatgaatacataaagataggaaggaattaattgttatatgtagtcatatgtctcttaatgacagggatactttctaagaaatacattgttaggtgattttgtcattgtgcaaacatcatagaatatacttacacaaaccttggtagtataacctactatacacctgggatatgtagtatagtctcttgccccagggatacaaacctgtacagtatgtaactgtactaatgactataaggcaattgttaacacaatggtaagttttgtgtgtctaaacctacacttgggctaccctaagtttatatatttttttaaatttctgttcaataataaattaaccttactttactgtaactttttaaactttttaatttttcctaacattttgacttttgtaatacagcttaaaacacacattatacagctatacaaatttttctttccttatatctttattctgtaagcttttttccatatttaaaattttttgtttgtttttacttattaaacttttttgttaaaaactaagacatgcatgcacattaacctaggcctacacagggtcaggaccatcaatatcattgtcttccacttccacatcttgtcccactggaagatcttcaggggcagtaacacacgtggagctgtcatctcctataataacattgccttcttttggaatacctcctgaaggacctatccaaggctgtttatagttaacttttttttttttttttttttttttttagtaaataggaggagtacactataaaataacaatataggtgctataccattatacaactgacagtgcagtaggtttgtttacaccagcatcaccacaaacacgtgagcaatgtgtcgtactacagtgttaggatggctataacatcactaagcaataggaacttttaaactccattataatcttatgggaccactatcacatatgcaatctcctgtggaccaaaatgtcattatgtggtacatgactgtactaagaaattgatccatctatattccatcaatttgtttagggctttttctggttacatttacctgtgagcccagaaaaccagttttgtagaaattaacttctgtaatgctaggagttaaaaaaaattgctgaacaacttttacattgttaaacatttaaaaacaagcgttctagaagtttatcaaatttcataaaggtgcaaaaatgtaaatgtaaatcattatccagctaatatatatgttgtatttccctagtaggagagcatatgtacctcttcctagttatacaaatttgatatatagtaaagaaacagtaaattctacttcaagtcattttgggaggattaaaaactgaatttctctagtttgaccattgtacagatttatctggcaattttactaaaacctgatttataggttaaacttggtgtatatcatatatcactttactttagaggaattaagatttcacataaatccatttccaggttccaaagaccaggaagaggcttggtttttgtttttctttttactgtctttacagtctccttgacttttcttaggagagaaggtactgagaaaacatgattctaatatttattattttttcttccaacattttcttatgaaacattttcaaatacaaaattgagttttatttaaaacatttgcaaatatactacctagattctaccattgttgttttatatttgctttacttacaacttttaaaagatgctttttataccactgaacattttagcttacatttcacaaagaaaagaaaaaatttaagagactttgcataatgttttaaggggttgcagtaaagaagtgcttcttatattttcttatgcatacaaatcagctgggcttattaaaatccagattctaattcagaaggtttaggtggggaccgagtctgcatttctaacaaactcctaggtggtatttttcttggtacttggaccatactttgagtagaaaagcagtagaggacataaaaagagtcttgttagtcccactttgttgctgtccacttctcatttgataatatcctaaaatagctgtgtctcctttttggtggttgtatgattactacctcagaagtactaattgattcttgctatttgaccttaatactttaatataacacagcattcatatttgatcagaaaactatctggcttccttttataagagatttttaggttttatacagttttgtggccttgggtttttttgtttgatttgtttttttgaaggtatataatatgtaagtagataaacaaatttgatttgtagacatttttatgtggatcatctaattaaaaatggagggatacagtatgaaagaatacttgtacttcttaacagagcactcaacctttcttttacatcctgtttcactgatgttattatgtaatttatgttgctaaactataaattagatatttaatttctgttctttgatttccttttattattaaatggacttgttgatttgcctagaaattaatttgcctttcaaaagtcttattaatcttcctccgttgaaattaatttgatatttgcatgcttctggaagactttaaagagctattccgagtaactgtagagattataaaatgaaatatgggaattttaataaattttacatctccagttactggtgaaaatgtcaagtcctcctttctgcagagtattttgttactcatctgttattcagcttatttatttatttatttatttatttatttttctttctttcttgttttttttttttgagacggagtcttgctttgtcgcccaggctggagtacagtggtgggatcttggctcactgcaggctccgcctcccgggttcacaccattcttctgcctcagcctcccaagtagctgggactacaggcacccgccaccatgccttgctaaatttttgtatttttagtagagacgggtttcactgtgttagccaggatggtctcgatctcttgacctcgtgatccacctgcctcggcctcccaaagtgctgggattacaggcatgagccaccgcgcctggcccttatttgttttttaaacaaaattagtgtgcatatccttgttgtattttatcggcaagttgttttatgccctaacttttggggtcttgatcatgagcctaaaacacgtaaacacccaaaaagaattatattccggttaaaggaacaaaacattcatttagaagttctcatccatgtaaatcagaggctggcaaatattttctgtaaagggccaagatagtaaatgttttaggctttgagggccacaagtggtatctgttgcatttttttttaattatgaccctttaaaatgcaaaaatcgttgttagcttgtgcatagtataaaaataggctggccgcatgctgtggctcatgcctgtaatcccagaaatgaggtgggaagccgaggtgggcacaccacctgaggtcaggagttcgaggccagcctggccaacgtggttgaaaccccgtctctactaaaaatacaaaacttagccaggcgtggtggcgggtgcctgttatcctggctactcaaggggctgaggcagtagaattgcttgaacctgagaggcagaggctgtagtgagcccagatcaagccagtgcacaccagcctggacgaccgagcgagactctgtctcaaaaaaaaaaaaaaaaggctgtggctgcatttggtccattggctgtaatatgctgattcctaattctctgggtaactttagtgtttgattagctactagaagttaggttaaacttttgtattttacaggctaactttaataatcttaaagtaaaacttaacatagttcatggaaaggaaatagaaattttaccctagtactcttttttttttttttttttttttttgaggcagagtctccctctgtcacccaggctggagtgcagtggtgggatcttggctgattgcaacctcctcctcctgggttcaagcaattcttgtgcctcagcctcccgagcagctgggactacaggcacgcaccaccacacctgactgatttttgtatttttagtagagacagggtttcgccatgttggccaggctggtcttgaactcctggcatcaagtgatcctcccatctgagcctcccagtgtgctgggattacagacgtgagtcactgtgcctggtctctagtattttttttttttttgagacggtctcactgttgccaggctggagtgcagtggcgcgatcctggctcactgcaacctccgcttcccggattcaagcgattttcctgcctcagcctcctgagtagctgggactatgggtgcacaccaccacgcccagctaatttttgtatttttagtagagacggggtttcaccatgttggccaatatggtctcaatctcttgacctcgtgatctgcccgtctcggcctcccaaagtgctgggattacaggcgtgagccactgtgcccagctgtactttttaagataagaattgcagggtatatatttttaccaacttaataacttataattttaaaaagctaattacttggctagaatataatgcgttacatattctttacactcagttcagtccatatctgaaaggcaaatagaattattttctgctagtacattgtgtagtccctatgttcctagtgtataaggactgttacctagttcacatttatctgggttgttgacagattttcctggtccctttggacagtgcatggccatgttggcaaaagctgtcaaaattgaaacattgacaccatgagaattgtgtgttttccagtctgctaaaatcaaaagtgggagggttcagtaaggtgaataacagaagcagagttttcggggtatctgttactcctcattcggcttttctgctctctgggggtctcaatttaaatataatgtgaaaattagttttacgaacctaaaaatgttgagtgattcatttcctggttttgttgttaatttctagatatttaaattaattgttagaagaaccccgttaaagaatgctttgcaaaacaacctccttatgtgctatgtctctgtttaatagtagttgagtttgtgtacatgagatcaatattttgaactatagctttttatgagttaaaaattgacggaacagttactgtgcacttgctgtgcaccatggtagtctcccaagtagtggtttttctgcatttcaatagtacatgagataggctgtgggtggcaaggtttcttgagaaagtgagggatgcacagttgggttttagaatacatcttgttcctccatgcccttccccaccaaaaggctggtagtcttgcatttgtatatagttagggtatttgatgtgttgcttccttgacagagttttgcaagaatttgcagatttaacaggaacaaaaacttacttaaaacaaaatctcttagtaaaagcatagtctagcaagatttagaatgatactttggctaacagtactttctctatatggagtgctttgtttccatagcctcacaagtatgttttcagataatagttgagttgaaaatgttgtcaatctcttgattttaaaaaatttacatatttaaagttgtatacttttgttcctacgtattttcagttgttcttaaagtttaataagtgacatttgaaaatgagtatatgtgtataaaaacaaaagtaggctaggcacggtggctcatgcctataatcctagcactttgggaggctgaggcaggcggatcacaaggtcaggagtttgagaccagcctgggcaatatggtgaaacccccctctactaaaaatacaaaaattagctgggtgtggtggtgcatgcctgtagtcccagctactcaggaggctgaggcaggagaatcgcttgaacccggaggtggcggttgcagtgagccgagattgcaccactgcagtccagcctgggcggcagagcgagactccatctcaaaaaaaaaaaacaaaaaaagaaaaagttaaaaaaaaacaaaaaacccccacaaaatgagtatatgtggcaacaagtcctattctcaaaaaaattattgtgtgctagttaagagcttaatgagtagccagtcggtattaaatatctgtttcagctatattttatctttaaaaattatctacagattttggaatgtgaaaaactagtgttttgtttcataggtatatactgtaggcattttaaaaataagagccagtgccagtggtttacagtgtacacaaggataatgttctcatgttctcttgatgtcagtatgactttaaagcatattatcaagaaataactaagtctgaaaaactgtggtaaataactggtactctaaaacctaagtttcttattactaaaaataagaaatggtaaaagtcaccctgtgctgttaattatatgagccactgaggtcctgacactgaattcttggtggtggataataatctcttctttttaattattggcttccaattctctctgcattgctggaaacaaaaatcatatatttcactattggtggtggggatgctgtcactgaaaaagtagacacattcatattgattttagaaataagttaaaatcaaaatttgcttctgctaaattagtagaggaccaatactgtttttctccttcatagtatgttttggtacttctacattgacattataacttttttttttttaaacagaaatagaagtttacattcttagaaaatttatgaaaatatgagcttttacctggtttgtgtgtgtgcgtatatatatacacatatttttaaatttcttacattgattttcaaattgaaagagaaccatttgtgaaagtatcttaacagagctcatgctttacattttacatgctacaaagttattttagtgccttaaattatttatgttgcttattaatgaaaattttggatacataattttttcaagacaaaggtaaaaataataaaccctttccttctgaggattaatgataaatataaactttaaaacgattaaaaaaatttttttagagacagggtcttgctctgttgcccagactgaagtgcagtggtgcagtcatagctcaatgaagcctcaaactcctgggcccaggcaaccctcctgcctcagccttttgagtagctgggacttcaggctcatgccaacatgcctaatttatcttatttttagtagagatgaggtctcaaactcctggcatctcttgccctctcaaagtgctggtactacaggcattagtcaccacacctgacacttaaaatcttttatatacaggtgtaagtgggtatctaacttaaagtgccaacgaatgtagttgaaagtttgtagttggcttagctaactagttaactaaattgattccattaaaaataagataagactgctcttagaatataatgatttttgttattcgttaaatataaatatatcactggatagtatatgttaatgacttgagatacgcattttaacatataatcacgttacttaaatgcctgcctttgaactgaaacttaacattatgaatttaaattaaagtttgactttagaggtaaatttctgtactttactaaagcagttcttaatataattctgagatttctaaaaattagtgtgccctaaagaattgaggtgtgtttttcttaactactgtaggcagtagatgtacagatgacttctgcatgcaaaaattaagccctagccattggtttacttcaactaatacttagttgccaattctctgtgtgtgattgaatttaaaactgcaaatggtactggtgatacattaactttttaggtgctaggtccactttgttacatttggttcagtagaaacattgatgttaccaatctcagaaagctaaaatatgtatgccaatccccaaattaggtaatttattcttaattttaagataaaagaatagaattcccttaaaattaaatgtggagtaaaatataccagctttaaaaaatattcacctttctgttagaagaatgaacataatattacatcttttaatttgcactatatatagattaatatttctgtgtatttctctgtgcccctactttgatggtatgcttttctgaacaaactagcagcacagttaactaagcactttgccccgtttgatgactgcctaattttctagattggaaaatattaaaaacttttatctccatatggccaatatatgattgtacctgttgtcatagctctcttatgtttaagcaagaaaaaccctattaagagtatttaaattagaatggaaggcacacagccagtatgattgaacactgttctaaaaattatttttaagacttgtagtaaggccaggtttggtggctcatggctgtaatcccagcccttaggaggccaaggtgggcggatcacttgtgctcaggagtttgagaccagcccgggcaacatggcaaaaccctgtctctacgaaaaatacaaaaatcagtcaggtgtggtggtgcttgcctgtagtcccagctatttgagaggctgaggcagggggatcacctagcctgggaggtcgaggctgcagtcatgatcgtgccattgcactccatcctgggcaacccagtgagaccctgtctctaaaacaaaaaaataaaaaaagaacttgtagtaaggatacaaaatgctcctattttgtgtgtgtcctttaattcatgatgtttttatattatggtaagcagctctcatttaagattttaataatgtaattaaacatgtacagaagacccagtctcagcttcacttgtataccctggaaatagactgaaaggtgttaaaatttaagataaaactcaaggttccagtttcttgactcacctttgagattcttttatgtttttgttgttttttaacaaaggtttcacgtccatattttaccatttttcttctcattctcccctggaggagggtgtgggaatcgatagtatataaatcacttttttcctaagtcaaagaagtaatttaaagctaacttcagtttaggctttaattccaggactagcaaactaaaatggttgcattaattgacaaacagatgctaatacctgtgtttaggcttgtcataatctctcctaattcctaatttaaaaattttaaaatttaattccattagaaaacaaaactgacttttaagaacaaaccaggattctagcccatattttaaaactgcatcctcagttttattcaaacagtctgatgtctgtttaaaaaaaaaaaaatctcaagctcataatctcaaacttcttgcacatggctttcccagtaaattactcttaccaatgcaacagactttaaagaagttgtgttttacaatgcagagagtggaggatgctttttatacattggtgagggagatccgacaatacagattgaaaaaaatcagcaaagaagaaaagactcctggctgtgtgaaaattaaaaaatgcattataatgtaatctggtaagtttaagttcagcacattaattttggcagaaagcagatgtcttttaaaggtaacaaggtggcaaccactttagaactacttaggtgtagtattctaacttgaagtattaaaagataagaaacttgtttccataattagtacatttatttttaatctagtgggaattaattataattgagacaattttgatggctgtagtagactaatctatatttggcataaagtctaatgatttaatgagtcttaagtaaactaaatatttggaaactgatatttacctttatttttaagggaaaagttttgagataatcagcagcttttttttttttttttttttttttagtagggagaaaaagatatgagctatagtagacagcagtaatattgaatggcccagaaggtgggaaaaagccactcttaaatgtattttttcttttggatattttacaagcaaataataacttctgcctaagttcgccatctcagtggcatcagcagcacagcactttcttatcccagtgagaaacctgggaattttaggatgactcctaccgccctcttttccccctggtttggaagtatccacaaattcctgtgacgttacattctgtgtcttttatgtcatcattagttcaggcccctatcatttcttgttggactgttagaacctcctatttggtttaccagttgctgccatcattcattgtgaaaccggagagatacactttaaagaaatgtcatttttggccgggcgcggtggctcacgcctgtaatcccagcactttgggaggcctaggcgggtgatcacctgaggtcaggagttcaagaccagcctggctaacatggtgaaaccctatttctactaaaaatacaaaaaattagccgggcgtggtggcacgtgcctgtaatcccagctacttgggaggctgaggcaggagaattgcttgaacctgggaggcagaggttgcagtgagctgagaatgcaccattgcactccagcttgagcaacaagagcgaaactctgtctcaaaaaaaaaaaaaaaaagtcattttagctatagaataaaatctcatgttccacatgtgttgcagatagtccttactaccttcccaccactccagctcttttttggtcttatatctaaaaacgtcatcttgcctgaatttcttttgttcttctataaataaataccatgttatttcctaccttcccttgagtcttggctcttgtttggaatgccagtatttttatccctagtcttactaattagctaacactctcatgattccccagtctcctactctctaaaaacctttctttaaacccttagactaggcatggagcccttcctgtgtattcccagaatactattcttaactattatatgcttcccatgttatgttgaaataactaacctcttctgtttcattcctatattacttgacagcaaaatcttagccagaattacatatttttaatctttgcacacccattgcctagtaaggttcctgggacatagtaactacccagtaaatatttattgcgtggaattctcattttcgtttctaaacccgtattaaactctgtcttgctcagaaaatacttcactaggtatcataaagttcatggcagagcttaagctttggatgcatattgtttgtaatatatcatgttcttaagaataggcaataaaattacagttttcaaaaactactacatttattatatttattacaagttggtgttctttattacatgaattttaggtatttcccaaaagtataaaatatacatttgaatagtagactcaatcccaaaagatactacgtggtgtactaatctactaaactcagaaacaaagcatgactggcattaatttttgttgaaatttatgaactctgaatgtttttgaatatcattctgtaaagcaatattttgcaattaaagcaattttgcatgttaaattttaccacaacctctaaaatattgcaaatttaacaatacagtttgaaaagttacacattttaaataacagtaccatgaccagatttaggtggtggttttaattttttattttctcctcctattgtctcaccattagatgattttaaaaatagaattgtttagagtaaaataagtgttatgctctaatttatatttaaaatgaaggtttaagcacgtactattctaaaatttctaatttgtgcaaattatgttttatacagtgactgtaggtgaatgtcacaattgtttgatgtgacgaatccttgtttttcagtacacgtggaagtaattcatataaaagagaagtatacttggtaattaaaaatttaaaattaaatacaatttaaaaaaaaatttatttgacaagctggctgtggtgtgtgtgcctgtagtatcagctgcttgggagcctgaggcaggaggattgcctgaccccaggagtttgaggttgaagggagctatgatggtgccatggcactgtagcctaggcaacagaaagagactccatctcttaaaaaaagtaaaaataaaaaaattttggcacagggacagtggctcacacttataatgccagaactttaggagtccacagcgcgaggactgcttgaggccaggagtttaagaccagactgggcaacgtaatgagaccccacctttaggaaataaatacataaataaaaatttgacaatgataaacatatataaattagcttttcttagtcctgaaaaagataatgttatgtgtatgtgtgagaatgattagttctcatatgagaaaaaaagaattcattgctctgtgtaggttgtgacatttccttcacgattgaaattaattaatttttttttattacttatttatttttaaaatagagacaggttcttgctgtgttgcccaggctggtctcaaactcctggcctcaagcagttctcctgcctcagcctcccaaattgctgtgactgtaggtgtgagccactgcactgggccaaaattacttaattttaacaagatgatgtagagaggagagttcattgcaacataagcctagaatctttgtcagaatcttaggaagtaatgttttcaaattctgtgttttcaccataaaatgtgtcttctctgtgtccatcacatggtttttcattgttttctgctttaccattttagtaccattggcatttttcttcattgtaaaagtagtagaaatggagtagattacataaggatgtgatcagagggaatttattcattcagggtaagggagttagatcctcttttaagattctatcacattctaagggtttatgattctaaactgtcaagtaaattgtcaagtgctggcaagctacagaataatttttattgtatcattggaaattttcccctctatatgtgttaaagagtttagcctgaagggatacatacacatacatatatgtaatcaaaccttgatggtattgtattgctgataaattatttcttaccacttttcctttctcctgtgggagaaacaaaagcatatgtttgtgtagtatcagtaatgatattagagagtgggaaacatcagtgagtgcagtttggggactttattggagactttcactagtgctcaaataaataatgctggtttttatcctactgtttgcttaatgtggactagcctcttattcccattctatgtttacctctcttaaaatattggtcacgctttcttgaattatagatctattaggaaaattcatgaactgtagctaattttcattgttcatgctccagatttattttgaaatatcgttaatcttagtagtacagtaaaggagaaataccacttaacattttttgtttttttttctttgagacagagtcatgctctgtcacccagtctggagtgcagtggtgctatctcggctcactgcaatgcacttcgcctctccgggttcagcaattctcctgcctcagcctcctgagtagctgggattacaggcacctgctaccacacccagctaatttttgtatttttagtagagacagggtttcaccatgttggccaggctggtctgaaactcctcacctcaagtgatccacccgtcttggcctcccaaagtgctgggattacaggcttgagccaccgcaccccgcccacttaacattttaaattaatttcaagataatatcacttgaatatttttacacatataatttttttaatacatttatttacacagtttataatatcctacaaagtgattacaatgagtaaaaacccagttttcattgttcctaaagtggcttgatttatacaacttaatgtgttgggtatttgtttctaagactccctctgctgtctaggtttggaagtattgtgaggttaacagattttctttttatagttactactcagttgaacaggctttaaaatacagagagaatcatattttttcttcattttttgcttttatttatatttttcttttaattggagacatgacaagaattgacttgtgtatggatcttgcataatttaagtactgcaggtttaaaatctactaccagtttgagagtgccatttttcacactgtagattattaggttgaaaagtattatggcttaaaatcgcttttagccattaaatttaaataaccttgctttaatcataaatagatggtggtcacaatgactaactgttaaactctttgaagacaggatatttggctttatatggcaagcttttgaatacaacagaaattaaaactttatgggatagaaagaatctcctccaaattggtaaactataagacctttcaaatgatttagctaatttctccacaaatctgaggtattagtgttttttttaaagtggtattctcctgtgttggggtcactttaaacctttttcttaatgataaatatatgaattgaaactaatcccttaatatatatcatttgaaaactgaaataatatgtttagatactgtttacttgttgataaattattggaataggatgttcgaatactgtttacttcttggtaaatttttaaatccaatggattttacgtaagtatagaactggagctcaaatactgttactgtgtgtgaagatatatgaacatagtttacagttgcatggcttatatctaaagtccagaaacataaggacaattaagtgtacacacacacacatgcatttggattttgatgacttaggtttgccaatgtggaaaaaatagtagcaaattaagttctcctgtgaaaaagtcgttaccttatttaaaattctgtgccattggttatccttgtcttttgtgaaaattagtgttcctgtttataatattgacaaaacacctatgcggatgacatttaagaattctaaaagtcctaatatatgtaatatatattcagttgcctgaagagaaacataaagaatcctttcttaatattttttccattaatgaaatttgttacctgtacacatgaagccatcgtatatattcacattttaatactttttatgtatttcagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 5 - DNA, 12438 bp, Homo Sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggtaagtttatctcatgcatagtgttcggctttgggctgtggaatgttcaggcgtttcactgatgccagaaatggagcagaatctatcagctggagacaaaggccttgggcgggggtccttccatttggtgcctacgtggggagatctttggagacagaagggagaatgggaaggagttgcggcctggaggcttcctgctagagctgagaagccttcggggagtaataggaagggggatttccattgcttaggctgagggcggggcccaaggactgttgaaaaatagctaaggatgggggttgctagaaaactactccagaagtgtgaggccgatattaatccggtgtttttgcgttctctagtcactttaagaaccaaatggaaggtcacactagggttttcatttccattgattatagaaagctttaaagtactgtagatgtggctcgccaattaaccctgattactggtttccaacaggttcttgctggtgtgaaatgactgagtacaaactggtggtggttggagcaggtggtgttgggaaaagcgcactgacaatccagctaatccagaaccactttgtagatgaatatgatcccaccatagaggtgaggcccagtggtagcccgctgacctgatcctgtctctcacttgtcggatcatctttacccatattctgtattaaaggaataagaggagagaaagtaaaaagttattttgggtatacattcagttatgcaataagcttaacgtgtttatagagaacagttcatttttattagctgctgaagtttctaaaacctgtccagtttttaacagttctgtaaactattgcaaactcagtgttgagttcattcatgagtttcttcatatataacagctctattacatgagaaacacaggccatagtagcgagactgtctgattgtatgggagataataggatggagataaaggattcagagatgagtgttcttcaatatttatttattagctagttgaagcagctgagaccagatgattggagtagcaagaacttgagatttttagtctttatgcctaggattttggtccctgtttgcagtttatttagttgtgtgatattgagcaactgaatctctcccaacctcattttcctcatgttttaaattaccataaacttgtcctgcctaccacacagggatgttatggaaagttaaataatatatttaagttatttatgaatggtaaagcactatgtaatagtacttagggattctattgttattatgagagttcatggtacagattgtcttcagtaagtggcacctaaggctctttaaataaagggttttgccggacacggtggctcacgcctgtaatcccagcactttgggaggctgaggcaggcggatcacaaggtcaggagttcaagaccagcctgatcaacatggtgaaaccccgtctctactaaaaatacaaaaattagctgggtgtggtggcaggcacctgtaatcccagctactcaggaggctgaggcaggagaatcgcttgaaccagaggcagagggtgcagtgagccgagatcacaccacagacctccagcctgggcaacagagcgagacttcgtctcaaaaaataaataaatagataaataaataaagggttttgtaattttgttcagtttagaaatgcctaactttagagattattttaatcaacacctggcctccctaccatctggctactcgtgtttaattgatgaaaactaactctaatgtagccactataaaaaattggttgctaacccttggcaaaatctttattttgagcttaacagctttaatattttacatgaaatgtttaatattttaattaaatatttttaaatgtttgatttattgagcaatttacataagtaaaatacataaattttatgtctacagcccagtgctttttgcgtttctatatagtcatgtagctaccacccagataacagtatagagcacttccagtactccagagagttctccaagtgtgatgacattaaaatacaagtaaaagtcctgttgccataaaaccaaaatgaaagtattttttatatgatctatgcatgtttgtcttcctgagaaattaaacataactataccttgtttggaacctttaagaatttgattcaggaatatttcccaaaggtacatctgtcatgataaaaaaaaaaccttctctgaaacaaaggtatttgtatatttagtcataaacacaaatgatgtatatagggccaggttataattggtggaggtatgtttagatttctttaagtaaaataaacagcacaaataaaacagtccagttcatagcttagtgaaatacactgggtacttaatctgtagcctcctggctgcagtagagttgtcatttgagttactgtgttttcttaatcttttccaggaacacagtgaccatatttcttttctgcaggcatatagaatttggtgggttttcttttatgtagggtgatattggatactttttgtttgtgattatatattagcaatttgagggacaaaccagataggcagaaatgggcttgaatagttagatgcttatttaaccttggcaatagcattgcattccctgtggtttttaataaaaattgaacttccctccctccctgcccccttaccctccacacccccaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacaggtactaggagcattattttctctgaaaggatgatctttgtgttctgaatctttatggggaaatgaggttaccacactagggaagatagagctttttaattatgggaagagttggttttaggttgtttgacattgagaatctagggtaattactgaaagttaatactggaatttattttacataatatactgttactataaagtttgataatacataagtgaagcttgctactgggaatgacttggaaccagagttgttgtaattagagatcacgaaggaatttcagagaggaaaacatctccaagaaacatctttcagtatgtaatggaaaagataggccaggcacagtggctcacacctggaatgtcagtgctttgggaggccaaggcgggaggatcactttcagcccaggagttggagaccagcctgggcaacagagcaagaccctgtctctacaaaaataaaaataaaaaaattagtcacacatggtggcagctactcgggaggcagaggtgggaggatcacgtgagctcaggaggtcgaggcatgctcactccactgcactgctgcactccagcctaatcaacagagcaagattctgtctccaaaaaaaataaaaaataaaatgataggagtaagcaaataggaagtccataaagatgaaaacaaagcaagggaacataaagatagactttgtccatagaaccataaagtttcaaagctagattggaccataaaaattctagtacaatattcttattttgcagaatcagaaacagagttcagaatgtcgtttgttaggttttggagtcaggattgttattagtagcagagccaggaccaaaaacccaaagctcctttttcttagcacagtgttcttaaacagaataatataatggttaagaatgagaactctgcctggattgaaacctagctctgtttattagcgacgtgactcaggggctatgtggctttcctaacctataatatggaaataataatacctacctcatagagttgtgaagattacagttttaataaatacgcaaatcactcagaatagtgcctggcacacagtaaatgctacttaagtgttctgcctaaaggcttgagtcttggcttattttctatccatgtgaagatgtctgctctcaaaagcagattggtccaacactgaattcaagtgttcttttcctaacctgttgtacttcccattttttttttgtctaaaagtaatagcagtacttaataaaatgcccacacttggcatgcatctaataaatgttttttgaatttctagaagtcatttttcttctttcttacaagaaatttattcatttttctatatgccttagctcaaaccaaagagtatttaaaacatcttatgaaaatgcatatagtagagcaagataagattaatgaaaaataggcttaagtgagaccaaaaaaataagggtaaaataaacaaatttaggagtgagcatattctgtgattgtgcatgaagttccagtggctttctaaaggtggactacaaatttggtcactgttgtgaaaaggaaagtagccagctgaaagattcagtatctgtatgctgaaagccgttaagttgctcggactagaaggaaattttcccatggatcctcaaagaggcttgtttaatgtaaaaatcagtagtaacctgacagtgacatggtccaggtactttaggctgtcttatcccttaatgtaggctattaccatcaagcacagttttgcaaatagccagtggaatgtagttcagatacatgactttgtgggtaatccaggggtagagactaaaacagtactgtgcagtatgtggttatttacagttaattaagattaaataaaatttaaaaattagttccttgactaccaaatgctcaatagccactagtaggtaccatgttgaacagtacagatatagaccatttccatcatcacataaagtactgttggattgtgttggtcaagacaatctaaagcaattgtttcccaggtgtgctgtgtggtgtgccttacatgtcattgaaaggggtgctgtcaggagttctagatgcttcagcctccctttactaagagcagttcttatgttttctattttatcgcttgggcttccagatacaatgttttgttttaggtttgttttttttgtttgtttgtttttttttttttgagacggagttttgctctggttgcccaagctggatggagtacggtggtgcgatcgcggctcactgcaacctccgcctcccgggttcgagagattctcctgcctcagcttcccgagtagctggaattacaggcgtccaccaccatgcctgactaattttttttgtatttttagtagacttggggtttcaccatgttggccgggctggtttcaaactcctgacctcaggtgatccacccgcctcggcctcccagagggctgggattacaggtgtgaaccaccgtgcccggcctgttttagttttttagagatggagtctccctctgttgcccaagccagagtgcggtggcatgacactctcagggttcaacctctcagggatcaagggatcctcccacctcagcttcctgagtagctggaaccacaggcacatgtgccaccatgcccagctaatttttgtattttttgtagaagcaaggtttcaccatgttgcccaggctggtctcggactcctaggtcaagtgatcctcccacctcaatctcctagagtgctaagactataggcgggagtcaccatgcccagcttcatctacaatttatttgaagaaaatgtttgagcaccacccatcttgaaaagtgatagactgccttccattaaatactgtcacacctagttatttagcagcagtgagcttcactttttatactttagaccttaatctaaagggtgatttctagttgccagttaaatccagagccaagctctttggagaatccaggagcctcactaggtcatgtatcaggataaaatacccatccactcccattagaaggtgagcttgtacttatggcttcctgatggctgctgcaacaagtctaaagcagtctccttagtatacaatgtcttctctaagtggtagaaaaaagcaaaaatactacaagttaatagggctacataaaatttgctagtttcttttttgccctagccatttattccttcctgaaatcttgtctctctctcgctctctctttctctcgctctcactttctttctctttttcttttctcttttcttttctttcttccctttcttttctttcttttttcctgttgcccaggctggagtgcagtggaacaattatggctcactgcagccttgacctttctggacccaggtgatcctcccacctcagtctcccaattagctgggactacaggcatgcgccaccacacccagctataatatatattgtatatatattttttatttataaatatatataaatatatatttatatgtgtaaattatatatatttatatattataaattatatataaatatatatttatatataatatatataatatatatttatttttatatattttatatatattttttggggggggttgggggggatggagtctcactctgtcgcccaggctagagtgtagtggcgtgatcttggctcactgcaatcttcgcctcccaggttcaagcgattctcctgcctcagcttcccgagtagctgggactataggcgcctgccaccacacctggctaatttttgtatttttagtagagatggggtttcaccatattggccaggctggtcttcaactcctgaccttgtgatctgcccacctcaacctcccaaagtgctgggaatacaggcatgagccactgcacccagcctaatctttgtatttttttgtagagaccgggttttgccatgttgcccaggctaatctcaaactcctgggttcaagcagtctgccctcctcagcctcccaaagtgctgagattgcaggcatgagccactgtacccagcctaatcttgtttttcttatgttctgataatatattcccgtttttagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacaggtatggtacagctttcagcatttgtgcaagagtttgcatcagttgattaactctggtagagatgtgatccatattcatattctttgttgttatgcatttttttcatttttatttttttattttttatttttttttaggcagagtctcactctatcttctaagctggagtgcagtggtgtgatctcagctcactgcagcctctgtctcttgggttcaagtgattctcctgcctcagcctcccaagtagctgggactacgggcacatcatcatgcccggctaatttttgtacttttagtagagacagggttttaccatgttgcccaggctggtctcgaactcctggcctcaagcaatcctcccacctgggcctcccagagtgctgggattacaggtgtgagccaccacgcccagcctgttgttaggcatttttagtagtgttctttttcttaacgcttgtttaaacccaaaatgaacttactaatattctgttatggcatgtttactcctgcattaacatccacaaatatttcttgggaagatccttgactaaaaatatttataaacattagttatttctctgtcaacaccagcccgtttatggcttaagcctcctgaatggagtctttagtttaatgtagttttgttccgtgtttctcacattacccttttcctttgcatgaatgtttatttggcaaaatgtgccatttttatatcagcctgttcttgtgattcaataggaatgtgaaatttagtgttctcttccttaaatcaccatattttattttatcagctattcgtttagtaattggaatcttatgtccacataaagagatacaaatgcaagagagcttataatttggattgtgtccgttgagctagctctctcattttttttcattttttccttttatagggtgttgaagatgctttttacacactggtaagagaaatacgccagtaccgaatgaaaaaactcaacagcagtgatgatgggactcagggttgtatgggattgccatgtgtggtgatgtaacaaggtgagcatatggtttcttggcataattacaaatcttagtatatagtattgggcaatttggaggagtgctggtgttattgtctatatgttttttgagtttctgcctatcctcttctgcacattttccatatgacaccctttctgaaagtactgaggtctaaagtgtttaaaacatttgattattccacaggtatctttatatttttggtaacattagaaattataagacattatttatgaaatgtaggcataccctattcctggcaatgaccaggaatttgaaggatcactactttgaaactagttaataaggacatggtttctgttctttttttacagatacttttaaagttttgtcagaaaagagccactttcaaggtaggacaagtttggaaatgtattctcattcctgttaattttgtatatttgtttttcttatactctgaatgtgtcacttatacaaattctgtttctatttcagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattcttatattca

SEQ ID NO: 6 - DNA, 3309 bp, Homo Sapiens
tgccctgcgcccgcaacccgagccgcacccgccgcggacggagcccatgcgcggggcgaaccgcgcgcccccgcccccgccccgccccggcctcggccccggccctggccccgggggcagtcgcgcctgtgaacggtgagtgcgggcagggatcggccgggccgcgcgccctcctcgcccccaggcggcagcaatacgcgcggcgcgggccgggggcgcggggccggcgggcgtaagcggcggcggcggcggcggcggcgggtgggtggggccgggcggggcccgcgggcacaggtgagcgggcgtcgggggctgcggcgggcgggggccccttcctccctggggcctgcgggaatccgggccccacccgtggcctcgcgctgggcacggtccccacgccggcgtacccgggagcctcgggcccggcgccctcacacccgggggcgtctgggaggaggcggccgcggccacggcacgcccgggcacccccgattcagcatcacaggtcgcggaccaggccgggggcctcagccccagtgccttttccctctccgggtctcccgcgccgcttctcggccccttcctgtcgctcagtccctgcttcccaggagctcctctgtcttctccagctttctgtggctgaaagatgcccccggttccccgccgggggtgcggggcgctgcccgggtctgccctcccctcggcggcgcctagtacgcagtaggcgctcagcaaatacttgtcggaggcaccagcgccgcggggcctgcaggctggcactagcctgcccgggcacgccgtggcgcgctccgccgtggccagacctgttctggaggacggtaacctcagccctcgggcgcctccctttagcctttctgccgacccagcagcttctaatttgggtgcgtggttgagagcgctcagctgtcagccctgcctttgagggctgggtcccttttcccatcactgggtcattaagagcaagtgggggcgaggcgacagccctcccgcacgctgggttgcagctgcacaggtaggcacgctgcagtccttgctgcctggcgttggggcccagggaccgctgtgggtttgcccttcagatggccctgccagcagctgccctgtggggcctggggctgggcctgggcctggctgagcagggccctccttggcaggtggggcaggagaccctgtaggaggaccccgggccgcaggcccctgaggagcgatgacggaatataagctggtggtggtgggcgccggcggtgtgggcaagagtgcgctgaccatccagctgatccagaaccattttgtggacgaatacgaccccactatagaggtgagcctggcgccgccgtccaggtgccagcagctgctgcgggcgagcccaggacacagccaggatagggctggctgcagcccctggtcccctgcatggtgctgtggccctgtctcctgcttcctctagaggaggggagtccctcgtctcagcaccccaggagaggagggggcatgaggggcatgagaggtaccagggagaggctggctgtgtgaactccccccacggaaggtcctgagggggtccctgagccctgtcctcctgcaggattcctaccggaagcaggtggtcattgatggggagacgtgcctgttggacatcctggataccgccggccaggaggagtacagcgccatgcgggaccagtacatgcgcaccggggagggcttcctgtgtgtgtttgccatcaacaacaccaagtcttttgaggacatccaccagtacaggtgaaccccgtgaggctggcccgggagcccacgccgcacaggtggggccaggccggctgcgtccaggcaggggcctcctgtcctctctgcgcatgtcctggatgccgctgcgcctgcagcccccgtagccagctctcgctttccacctctcagggagcagatcaaacgggtgaaggactcggatgacgtgcccatggtgctggtggggaacaagtgtgacctggctgcacgcactgtggaatctcggcaggctcaggacctcgcccgaagctacggcatcccctacatcgagacctcggccaagacccggcaggtgaggcagctctccaccccacagctagccagggacccgccccgccccgccccagccagggagcagcactcactgaccctctcccttgacacagggcagccgctctggctctagctccagctccgggaccctctgggaccccccgggacccatgtgacccagcggcccctcgcgctgtaagtctcccgggacggcagggcagtgagggaggcgagggccggggtctgggctcacgccctgcagtcctgggccgacacagctccggggaaggcggaggtccttggggagagctgccctgagccaggccggagcggtgaccctggggcccggcccctcttgtccccagagtgtcccacgggcacctgttggttctgagtcttagtggggctactggggacacgggccgtagctgagtcgagagctgggtgcagggtggtcaaaccctggccagacctggagttcaggagggccccgggccaccctgacctttgaggggctgctgtagcatgatgcgggtggccctgggcacttcgagatggccagagtccagcttcccgtgtgtgtggtgggcctggggaagtggctggtggagtcgggagcttcgggccaggcaaggcttgatcccacagcagggagcccctcacccaggcaggcggccacaggccggtccctcctgatcccatccctcctttcccagggagtggaggatgccttctacacgttggtgcgtgagatccggcagcacaagctgcggaagctgaaccctcctgatgagagtggccccggctgcatgagctgcaagtgtgtgctctcctgacgcaggtgagggggactcccagggcggccgccacgcccaccggatgaccccggctccccgcccctgccggtctcctggcctgcggtcagcagcctcccttgtgccccgcccagcacaagctcaggacatggaggtgccggatgcaggaaggaggtgcagacggaaggaggaggaaggaaggacggaagcaaggaaggaaggaagggctgctggagcccagtcaccccgggaccgtgggccgaggtgactgcagaccctcccagggaggctgtgcacagactgtcttgaacatcccaaatgccaccggaaccccagcccttagctcccctcccaggcctctgtgggcccttgtcgggcacagatgggatcacagtaaattattggatggtcttga

SEQ ID NO: 7 - RNA, 110 nt, Artificial sequence
  Synthesized hsa-mir181a-1 miRNA
ugaguuuugagguugcuucagugaacauucaacgcugucggugaguuuggaauuaaaaucaaaaccaucgaccguugauuguacccuauggcuaaccaucaucuacucca

SEQ ID NO: 8 - RNA, 110 nt, Artificial sequence
  Synthesized hsa-miR181-a2  miRNA
agaagggcuaucaggccagccuucagaggacuccaaggaacauucaacgcugucggugaguuugggauuugaaaaaaccacugaccguugacuguaccuugggguccuua

SEQ ID NO: 9 - RNA, 110 nt, Artificial sequence
  Synthesized hsa-miR181-b1 miRNA
ccugugcagagauuauuuuuuaaaaggucacaaucaacauucauugcugucgguggguugaacuguguggacaagcucacugaacaaugaaugcaacuguggccccgcuu

SEQ ID NO: 10 - RNA, 89 nt, Artificial sequence
  Synthesized hsa-miR181-b2 miRNA
cugauggcugcacucaacauucauugcugucgguggguuugagucugaaucaacucacugaucaaugaaugcaaacugcggaccaaaca

SEQ ID NO: 11 - RNA, 110 nt, Artificial sequence
  Synthesized hsa-miR181-c miRNA
cggaaaauuugccaaggguuugggggaacauucaaccugucggugaguuugggcagcucaggcaaaccaucgaccguugaguggacccugaggccuggaauugccauccu

SEQ ID NO: 12 - RNA, 137 nt, Artificial sequence
  Synthesized hsa-miR181-d miRNA
guccccuccccuaggccacagccgaggucacaaucaacauucauuguugucgguggguugugaggacugaggccagacccaccgggggaugaaugucacuguggcugggccagacacggcuuaaggggaauggggac

SEQ ID NO: 13 - RNA, 106 nt, Artificial sequence
  Synthesized hsa-miR143 miRNA
gcgcagcgcccugucucccagccugaggugcagugcugcaucucuggucaguugggagucugagaugaagcacuguagcucaggaagagagaaguuguucugcagc

(19) United States
(12) Patent Application Publication
(10) Pub. No.: US 2015/0148244 A1
(43) Pub. Date: May 28, 2015

(54) METHODS FOR DETERMINING RESPONSIVENESS TO A DIHYDROFOLATE REDUCTASE INHIBITOR

(71) Applicants: Sarah Bacus, Hinsdale, IL (US); Diarmuid Moran, Westmont, IL (US)

(72) Inventors: Sarah Bacus, Hinsdale, IL (US); Diarmuid Moran, Westmont, IL (US)

(73) Assignee: Targeted Molecular Diagnostics, LLC, Westmont, IL (US)

(21) Appl. No.: 14/343,682

(22) PCT Filed: Sep. 7, 2012
(86) PCT No.: PCT/US12/54248

§ 371 Date: Apr. 25, 2014

Related U.S. Application Data
(60) Provisional application No. 61/532,318, filed on Sep. 8, 2011.

(51) Int. Cl. C12Q 1/68

(52) U.S. Cl. CPC C12Q 1/6886; C12Q 2600/178; C12Q 2600/158; C12Q 2600/136
USPC 506/9; 435/6.13; 435/6.12; 435/6.11

(57) ABSTRACT

The present disclosure provides methods for predicting or determining the sensitivity of a cell and/or biological sample obtained from a subject to a DHFR inhibitor including, for example, an antifolate such as Methotrexate or Pemetrexed. Such methods may comprise determining the expression level of one or more miRNAs (e.g., one or more members of the miR-181 and/or miR-143 families) in a cell or biological sample. The methods may be used to determine the responsiveness of a subject to treatment with a DHFR inhibitor.

### FIELD

The present disclosure provides methods for determining the sensitivity (e.g., responsiveness) of a cell and/or biological sample to a DHFR inhibitor such as Methotrexate or Pemetrexed by determining the expression of one or more miRNAs in the cell and/or biological sample.

### BACKGROUND

Folate (folic acid) is a vitamin that is essential for the life-sustaining processes of DNA synthesis, replication, and repair. Folate is also important for protein biosynthesis, another process that is central to cell viability. The pteridine compound, Methotrexate (MTX), is structurally similar to folate and as a result can bind to the active sites of a number of enzymes that normally use folate as a coenzyme for biosynthesis of purine and pyrimidine nucleotide precursors of DNA and for interconversion of amino acids during protein biosynthesis. Despite its structural similarity to folic acid, Methotrexate cannot be used as a cofactor by enzymes that require folate, and instead competes with the folate cofactor for enzyme binding sites, thereby inhibiting protein and DNA biosynthesis and, hence, cell division.

The ability of the folate antagonist Methotrexate to inhibit cell division has been exploited in the treatment of a number of diseases and conditions that are characterized by rapid or aberrant cell growth such as cancer and autoimmune disease. As an example, autoimmune diseases are characterized by an inappropriate immune response directed against normal autologous tissues and mediated by rapidly replicating T-cells or B-cells. Autoimmune diseases that have been treated with Methotrexate include, without limitation, rheumatoid arthritis and other forms of arthritis, psoriasis, multiple sclerosis, the autoimmune stage of diabetes mellitus (juvenile-onset or Type 1 diabetes), autoimmune uveoretinitis, myasthenia gravis, autoimmune thyroiditis, and systemic lupus erythematosus. A major drawback of Methotrexate therapy is inter-patient variability in the clinical response (Weinblatt et al., Arthritis Rheum. 37:1492-1498 (1994); and Walker et al, Arthritis Rheum. 36:329-335 (1993)). Thus, there exists a need for methods that can predict those patients likely to respond to treatment with an antifolate and methods to determine if patients treated with an antifolate are responding to the therapy.

### SUMMARY

The present disclosure provides methods for predicting or determining the sensitivity (e.g., clinical responsiveness) of a cell and/or biological sample to a DHFR inhibitor including, for example, an antifolate such as Methotrexate or Pemetrexed. Such methods may comprise determining the presence or absence (e.g., expression level) of one or more microRNAs (e.g., one or more miR-181 and/or miR-143 gene family members) in a cell or biological sample. The methods may be used to determine if a subject is responding to treatment with a DHFR inhibitor including, an antifolate such as Methotrexate or Pemetrexed.

The present disclosure provides methods for predicting and/or determining sensitivity of a test cell to a dihydrofolate reductase (DHFR) inhibitor, the method comprising: contacting the test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to predict and/or determine sensitivity of the test cell to the DHFR inhibitor, wherein the test cell has mutated RAS and no amplification of the RAS gene.

In some embodiments of each or any of the above or below mentioned embodiments, the miRNA is from the miR181 family (e.g., miR181c) and/or the miR143 family.

In some embodiments of each or any of the above or below mentioned embodiments, the DHFR inhibitor is an antifolate such as Methotrexate or Pemetrexed.

In some embodiments of each or any of the above or below mentioned embodiments, RAS is KRAS (SEQ ID NO: 1), NRAS (SEQ ID NO: 2) or HRAS (SEQ ID NO: 3). In some embodiments, the KRAS mutations are at one or more of positions 12, 13 or 61. In some embodiments, the KRAS mutations are selected from the group consisting of: G12A, G12N, G12R, G12C, G12S, G12V, G13N and Q61H. In some embodiments, the HRAS or NRAS mutations are at one or more of positions 12, 13 or 61.

In some embodiments of each or any of the above or below mentioned embodiments, the test cell is from a tumor biopsy. In some embodiments, the test cell is from an aspirate, blood, or serum. In some embodiments, the test cell is from a cancer patient.

In some embodiments of each or any of the above or below mentioned embodiments, the test cell is predicted and/or determined to be sensitive to the DHFR inhibitor where expression of the one or more miRNAs in the test cell is elevated as compared to expression of the one or more miRNAs in the control cell or is above the threshold.

In some embodiments of each or any of the above or below mentioned embodiments, the step of assaying the test cell for expression of one or more miRNAs is performed by in situ hybridization (ISH), Northern blot, qRT-PCR or microarray analysis.

In some embodiments of each or any of the above or below mentioned embodiments, the threshold is set at a level of miRNA expression above which the control cell is known to be sensitive to treatment with the DHFR inhibitor and below which the control cell is known to not be sensitive to treatment with the DHFR inhibitor.

In some embodiments of each or any of the above or below mentioned embodiments, the control cell is the same cell type as the test cell. In some embodiments, the control cell is a different cell type than the test cell.

In some embodiments of each or any of the above or below mentioned embodiments, the threshold is set at a level of miRNA expression above which 50%, 60%, 70%, 80%, 90%, or 95% of control cells respond to treatment with the DHFR inhibitor. In some embodiments, the threshold is set at a level of miRNA expression below which 50%, 60%, 70%, 80%, 90%, or 95% of control cells do not respond to treatment with the DHFR inhibitor.

The present disclosure also provides methods for screening one or more dihydrofolate reductase (DHFR) inhibitors for a pharmacological activity, comprising: contacting a test cell with the one or more DHFR inhibitors; assaying the contacted test cell for expression of one or more miRNAs; determining if expression of the one or more miRNAs in the test cell is elevated or reduced compared to a control cell or above or below a threshold; and employing the determination of the expression of the one or more miRNAs in the test cell to determine a pharmacological activity of the DHFR inhibitor, wherein the test cell has mutated RAS and no amplification of the RAS gene. Optionally, the methods may further comprise obtaining the test cell.

The present disclosure also provides methods of identifying a dihydrofolate reductase (DHFR) inhibitor that inhibits Ras activity, comprising, contacting the test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to determine if the DHFR inhibitor inhibits Ras activity, wherein the test cell has mutated RAS and no amplification of the RAS gene. Optionally, the methods may further comprise obtaining the test cell.

The present disclosure also provides methods of identifying a dihydrofolate reductase (DHFR) inhibitor that is toxic to a test cell, comprising, contacting a test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to determine if the DHFR inhibitor is toxic to the cell, wherein the test cell has mutated RAS and no amplification of the RAS gene. Optionally, the methods may further comprise obtaining the test cell.

The present disclosure also provides methods for predicting and/or determining responsiveness of a subject with a disease or disorder to a dihydrofolate reductase (DHFR) inhibitor, the method comprising: administering a DHFR inhibitor to the subject; obtaining a biological sample from the subject; assaying the biological sample for expression of one or more miRNAs; determining if expression of the one or more miRNAs in the biological sample is elevated or reduced compared to a control sample or above or below a threshold; and employing the determination of the expression of the one or more miRNAs in the biological sample to predict and/or determine responsiveness of the subject to the DHFR inhibitor, wherein the biological sample comprises cells with mutated RAS and no amplification of the RAS gene.

In some embodiments of each or any of the above or below mentioned embodiments, the miRNA is from the miR181 family (e.g., miR181c) and/or the miR143 family.

In some embodiments of each or any of the above or below mentioned embodiments, the DHFR inhibitor is an antifolate such as Methotrexate or Pemetrexed.

In some embodiments of each or any of the above or below mentioned embodiments, RAS is KRAS (SEQ ID NO: 1), NRAS (SEQ ID NO: 2) or HRAS (SEQ ID NO: 3). In some embodiments, the KRAS mutations are at one or more of positions 12, 13 or 61. In some embodiments, the KRAS mutations are selected from the group consisting of: G12A, G12N, G12R, G12C, G12S, G12V, G13N and Q61H. In some embodiments, the HRAS or NRAS mutations are at one or more of positions 12, 13 or 61.

In some embodiments of each or any of the above or below mentioned embodiments, the biological sample is from a tumor biopsy or blood sample. In some embodiments, the biological sample is from an aspirate. In some embodiments, the biological sample is from a cancer patient.

In some embodiments of each or any of the above or below mentioned embodiments, the subject is predicted and/or determined to be sensitive to the DHFR inhibitor where expression of the one or more miRNAs in the biological sample is elevated as compared to expression of the one or more miRNAs in the control sample or is above the threshold.

In some embodiments of each or any of the above or below mentioned embodiments, the step of assaying the biological sample for expression of one or more miRNAs is performed by in situ hybridization (ISH), Northern blot, qRT-PCR or microarray analysis.

In some embodiments of each or any of the above or below mentioned embodiments, the threshold is set at a level of miRNA expression in the control sample above which a subject is known to be sensitive to treatment with the DHFR inhibitor and below which a subject is known to not be sensitive to treatment with the DHFR inhibitor.

In some embodiments of each or any of the above or below mentioned embodiments, the threshold is set at a level of miRNA expression above which 50%, 60%, 70%, 80%, 90%, or 95% of subject(s) respond to treatment with the DHFR inhibitor. In some embodiments, the threshold is set at a level of miRNA expression below which 50%, 60%, 70%, 80%, 90%, or 95% of subject(s) do not respond to treatment with the DHFR inhibitor.

In some embodiments of each or any of the above or below mentioned embodiments, the disease or disorder is cancer. In some embodiments, the cancer is selected from the group consisting of gastrointestinal, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

The present disclosure also provides methods for treating a subject with a disease or disorder, the method comprising: administering a DHFR inhibitor to the subject; obtaining a biological sample from a subject; assaying the biological sample for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and administering to the subject a therapeutically effective amount of DHFR inhibitor where the subject is predicted and/or determined to be responsive to the DHFR inhibitor, wherein the biological sample comprises cells with mutated RAS and no amplification of the RAS gene.

In some embodiments of each or any of the above or below mentioned embodiments, the miRNA is from the miR181 family (e.g., miR181c) and/or the miR143 family.

In some embodiments of each or any of the above or below mentioned embodiments, the DHFR inhibitor is an antifolate such as Methotrexate or Pemetrexed.

In some embodiments of each or any of the above or below mentioned embodiments, RAS is KRAS (SEQ ID NO: 1), NRAS (SEQ ID NO: 2) or HRAS (SEQ ID NO: 3). In some embodiments, the KRAS mutations are at one or more of positions 12, 13 or 61. In some embodiments, the KRAS mutations are selected from the group consisting of: G12A, G12N, G12R, G12C, G12S, G12V, G13N and Q61H. In some embodiments, the HRAS or NRAS mutations are at one or more of positions 12, 13 or 61.

In some embodiments of each or any of the above or below mentioned embodiments, the biological sample is from a tumor biopsy or blood sample. In some embodiments, the biological sample is from an aspirate. In some embodiments, the biological sample is from a cancer patient.

In some embodiments of each or any of the above or below mentioned embodiments, the subject is predicted and/or determined to be sensitive to the DHFR inhibitor where expression of the one or more miRNAs in the biological sample is elevated as compared to expression of the one or more miRNAs in the control sample or is above the threshold.

In some embodiments of each or any of the above or below mentioned embodiments, the step of assaying the test cell for expression of one or more miRNAs is performed by in situ hybridization (ISH), Northern blot, qRT-PCR or microarray analysis.

In some embodiments of each or any of the above or below mentioned embodiments, the threshold is set at a level of miRNA expression in a control sample above which a subject is known to be sensitive to treatment with the DHFR inhibitor and below which a subject is known to not be sensitive to treatment with the DHFR inhibitor.

In some embodiments of each or any of the above or below mentioned embodiments, the threshold is set at a level of miRNA expression above which 50%, 60%, 70%, 80%, 90%, or 95% of subject(s) respond to treatment with the DHFR inhibitor. In some embodiments, the threshold is set at a level of miRNA expression below which 50%, 60%, 70%, 80%, 90%, or 95% of subject(s) do not respond to treatment with the DHFR inhibitor.

In some embodiments of each or any of the above or below mentioned embodiments, the disease or disorder is cancer. In some embodiments, the cancer is selected from the group consisting of gastrointestinal, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments of each or any of the above or below mentioned embodiments, the subject is a cancer patient.

In some embodiments of each or any of the above or below mentioned embodiments, the therapeutically effective amount of one or more DHFR inhibitors are optionally adapted for a co-treatment with radiotherapy or radio-immunotherapy.

The present disclosure also provides methods for selecting subjects for a clinical trial for testing the efficacy or safety of a dihydrofolate reductase (DHFR) inhibitor, the method comprising: administering a DHFR inhibitor to the subject; obtaining a biological sample from the subjects; assaying the biological samples obtained from the subjects for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and selecting subjects for inclusion in a clinical trial that are predicted and/or determined to be responsive to the DHFR inhibitor, wherein the biological sample comprises one or more cells with mutated RAS and no amplification of the RAS gene.

In some embodiments of each or any of the above or below mentioned embodiments, the miRNA is from the miR181 family (e.g., miR181c) and/or the miR143 family.

In some embodiments of each or any of the above or below mentioned embodiments, the DHFR inhibitor is an antifolate such as Methotrexate or Pemetrexed.

In some embodiments of each or any of the above or below mentioned embodiments, RAS is KRAS (SEQ ID NO: 1), NRAS (SEQ ID NO: 2) or HRAS (SEQ ID NO: 3). In some embodiments, the KRAS mutations are at one or more of positions 12, 13 or 61. In some embodiments, the KRAS mutations are selected from the group consisting of: G12A, G12N, G12R, G12C, G12S, G12V, G13N and Q61H. In some embodiments, the HRAS or NRAS mutations are at one or more of positions 12, 13 or 61.

In some embodiments of each or any of the above or below mentioned embodiments, the biological sample is from a tumor biopsy or blood sample. In some embodiments, the biological sample is from an aspirate. In some embodiments, the biological sample is from a cancer patient.

In some embodiments of each or any of the above or below mentioned embodiments, the subject is predicted and/or determined to be sensitive to the DHFR inhibitor where expression of the one or more miRNAs in the biological sample is elevated as compared to expression of the one or more miRNAs in the control sample or is above the threshold.

In some embodiments of each or any of the above or below mentioned embodiments, the step of assaying the test cell for expression of one or more miRNAs is performed by in situ hybridization (ISH), Northern blot, qRT-PCR or microarray analysis.

In some embodiments of each or any of the above or below mentioned embodiments, the threshold is set at a level of miRNA expression in a control sample above which a subject is known to be sensitive to treatment with the DHFR inhibitor and below which a subject is known to not be sensitive to treatment with the DHFR inhibitor.

In some embodiments of each or any of the above or below mentioned embodiments, the threshold is set at a level of miRNA expression above which 50%, 60%, 70%, 80%, 90%, or 95% of subject(s) respond to treatment with the DHFR inhibitor. In some embodiments, the threshold is set at a level of miRNA expression below which 50%, 60%, 70%, 80%, 90%, or 95% of subject(s) do not respond to treatment with the DHFR inhibitor.

In some embodiments of each or any of the above or below mentioned embodiments, the disease or disorder is cancer. In some embodiments, the cancer is selected from the group consisting of gastrointestinal, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In some embodiments of each or any of the above or below mentioned embodiments, the subject is a cancer patient.

In some embodiments of each or any of the above or below mentioned embodiments, the method may further comprise seeking regulatory approval for the DHFR inhibitor.

In some embodiments of each or any of the above or below mentioned embodiments, the clinical trial is a phase I, phase II, phase III or phase IV clinical trial.

### BRIEF DESCRIPTION OF THE DRAWINGS

The foregoing summary, as well as the following detailed description of the disclosure, will be better understood when read in conjunction with the appended figures. For the purpose of illustrating the disclosure, shown in the figures are embodiments which are presently preferred. It should be understood, however, that the disclosure is not limited to the precise arrangements, examples and instrumentalities shown.

FIG. 1 shows genes in the one carbon pool by folate pathway with significant expression differences between KRASmut and KRASwt NSCLC cell lines (significance p<0.05) (A). Expression of DHFR was determined by qPCR in KRASmut (A549 and H460) cells and KRASwt (H661 and H2126) cells (B). Gene expression was normalized to internal β-2 microglobulin control and expressed as fold change versus H2126 expression.

FIG. 2 shows a Southern blot for KRAS, DHFR, and TYMS expression in A549 cells transfected with an expression vector containing KRAS ORF (pcDNA KRAS) or an empty control vector (pcDNA3.1) (A). A quantitative PCR for DHFR, KRAS, or TYMS expression in A549 cells transfected with siRNA targeting KRAS (siKRAS) or non-targeting control (siCTRL) was also performed. Gene expression was normalized to internal β-2 microglobulin control and expressed as fold change versus siCTRL (B).

FIG. 3 shows a Southern blot for KRAS, DHFR, TYMS, E2F-1, pRb, and Rb expression in lysates obtained at 24, 48 or 72 hours from KRAS mutant (A549 and H460) and KRAS wild-type (H2126 and H661) NSCLC cells lines treated with Methotrexate (M) or vehicle control (V).

FIG. 4 shows the IC50 (μM) for KRAS mutant (KRASmut), KRAS mutant-amplified (KRASmut-amp), NRAS mutant (NRASmut), and KRAS wild-type (KRASwt) NSCLC cell lines treated with Methotrexate (A) or Pemetrexed (B).

FIG. 5 shows that KRAS mutant NSCLC cell lines treated with Methotrexate, Trimetrexate, or Baker's antifolate display higher sensitivity to anti-folate drugs compared to their wild-type counterparts.

FIG. 6 shows a diagram of cell cycle progression in representative KRAS mutant (A549 and H460) and KRAS wild-type (H2126 and H661) NSCLC cells lines treated with Methotrexate (M) or DMSO (V) as control. Cells in each phase are represented as a percentage of the total live cell population.

FIGS. 7A-C show a qPCR analysis of KRAS gene expression in KRASmut non-amplified (A549 and H460) cells (A), KRASwt (H2126 and H661) cells (B), and KRASmut amplified (H2009 and H727) cells (C) that were treated with 0.1 μM Methotrexate (MTX), 0.1 μM Pemetrexed (PEM), or control (Vehicle). Gene expression was normalized to internal β-2 microglobulin control and expressed as fold change versus untreated control.

FIG. 8 shows a microRNA analysis for RNA harvested at 24 and 48 hours from a representative KRAS mutant (H460) NSCLC cell line treated with Methotrexate (MTX).

FIG. 9 shows the GI₅₀ (μL) for KRAS mutant versus KRAS wild type NCI-60 NSCLC cell lines treated with antifolates such as Methotrexate, Trimetrexate, soluble bakers antifolate, or 5-fluorouracil.

FIG. 10 shows a growth curve of KRAS mutant cells (KRASmut), KRAS mutant and KRAS amplified cells (KRASmut-amp), and KRAS wild type cells (KRASwt) treated with Methotrexate.

FIG. 11 shows a Southern blot for basal KRAS expression in KRAS mutant cells that harbor a KRAS amplification (H2009 and H727) or do not harbor a KRAS amplification (H460 and A549) (A). The relative change in KRAS mRNA expression levels in KRAS mutant cells that harbor a KRAS amplification (H2009 and H727) or do not harbor a KRAS amplification (H460 and A549) was determined 48 hours after treatment with Methotrexate or Pemetrexed (B).

FIG. 12 shows the in vivo responsiveness of KRAS mutant tumors to Methotrexate.

FIG. 13: shows chest CT-PET Scans (Case 1 & 2) from patients diagnosed with NSCLC harboring KRAS mutation (A-J).

### DETAILED DESCRIPTION

Receptor tyrosine kinase inhibitors including, for example, EGFR inhibitors (e.g. Erlotinib or Gefitinib) are often used in the treatment of diseases and/or disorders such as cancer. However, several recent clinical studies have shown that the presence of a RAS mutation, such as KRAS, is a significant predictor of non-responsiveness (e.g., resistance) to treatment with an EGFR inhibitor. As such, targeted therapy options for KRAS mutant positive patients are limited. Thus, biomarkers and methods are desired which can be used to predict and/or determine whether patients predicted to be non-responsive to an EGFR targeted therapy are responsive to treatment with other targeted therapies including, for example, a dihydrofolate reductase (DHFR) inhibitor.

The inventors of the instant disclosure have unexpectedly demonstrated that cells which harbor a KRAS mutation (termed KRASmut) and do not have an amplification of the KRAS gene (e.g., have two or fewer, three or fewer, four or fewer, five or fewer, six or fewer, or seven or fewer copies of the KRAS gene, termed herein “non-amplified”) are likely to respond (e.g., are sensitive to) to a dihydrofolate reductase (DHFR) inhibitor (e.g., Methotrexate or Pemetrexed (ALTIMA™)) as compared to cells which harbor a KRAS mutation and have an amplification of the KRAS gene (e.g., have three or more, four or more, five or more, six or more, seven or more, eight or more, nine or more, or ten or more copies of the KRAS gene, termed herein “amplified”), or cells which harbor wild type KRAS irrespective of whether or not the cells have an amplification of the KRAS gene (e.g., have two copies of the KRAS gene or have three or more copies of the KRAS gene). Thus, contrary to conventional wisdom, mutation of RAS is not a sole predictor of resistance to a targeted therapy or chemotherapy. Instead, prediction of responsiveness to a DHFR inhibitor, including, for example, an antifolate such as Methotrexate or Pemetrexed, requires an assessment of both KRAS mutation status and KRAS amplification status. Moreover, the inventors have surprisingly found that certain microRNAs (miRNA) gene families including, for example, the miR-181 and miR143 families (e.g., miR-181c and/or miR143, respectively), that are validated herein to bind the 3′ untranslated region of human KRAS, are upregulated in cells harboring a KRAS mutation and no KRAS amplification (i.e., KRAS mutant, non-amplified) and that such cells are sensitive to treatment with DHFR inhibitors. Without wising to be bound by theory, it is believed that cells which harbor a KRAS mutation and no KRAS amplification are sensitive to treatment with a DHFR inhibitor due to the upregulation of certain microRNAs that bind to KRAS. Accordingly, the upregulation of microRNAs specific for KRAS in KRAS mutation/non-amplified cells may be used to determine responsiveness to treatment with a DHFR inhibitor. The present methods and materials may also be used to select subjects for inclusion/exclusion in a clinical trial, predict the responsiveness of a subject to a drug (e.g., a DHFR inhibitor) and/or select a drug that will elicit a response in a subject.

The present disclosure provides methods for predicting and/or determining sensitivity of a test cell to a dihydrofolate reductase (DHFR) inhibitor (e.g., an antifolate such as Methotrexate or Pemetrexed (ALTIMA™)), the method comprising: contacting a test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to predict and/or determine sensitivity of the test cell to the DHFR inhibitor, wherein the test cell has mutated RAS and no amplification of the RAS gene. Optionally, the methods may further comprise obtaining the test cell. The test cell may be predicted or determined to be sensitive to the DHFR inhibitor where expression of the one or more miRNAs (e.g., a member of the miR181 and/or miR143 families such as miR-181c or miR143) in the test cell is elevated as compared to expression of the one or more miRNAs in the control cell or is above the threshold. The threshold may be set at a level of miRNA expression above which a control cell is known to be sensitive to treatment with the DHFR inhibitor and below which a control cell is known to not be sensitive to treatment with the DHFR inhibitor. In some embodiments, the threshold may be set at a level of miRNA expression above which 50%, 60%, 70%, 80%, 90%, or 95% of control cells respond to treatment with a DHFR or at a level of miRNA expression below which 50%, 60%, 70%, 80%, 90%, or 95% of control cells do not respond to treatment with a DHFR inhibitor.

The present disclosure also provides methods of identifying a dihydrofolate reductase (DHFR) inhibitor that inhibits Ras activity, comprising, contacting the test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to determine if the DHFR inhibitor inhibits Ras activity, wherein the test cell has mutated RAS and no amplification of the RAS gene. Optionally, the methods may further comprise obtaining the test cell.

The present disclosure also provides methods of identifying a dihydrofolate reductase (DHFR) inhibitor that is toxic to a test cell, comprising, contacting a test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to determine if the DHFR inhibitor is toxic to the cell, wherein the test cell has mutated RAS and no amplification of the RAS gene. Optionally, the methods may further comprise obtaining the test cell.

The present disclosure also provides methods for predicting and/or determining responsiveness of a subject with a disease or disorder to a dihydrofolate reductase (DHFR) inhibitor, the method comprising: administering a DHFR inhibitor to the subject; obtaining a biological sample from the subject; assaying the biological sample for expression of one or more miRNAs; determining if expression of the one or more miRNAs in the biological sample is elevated or reduced compared to a control sample or above or below a threshold; and employing the determination of the expression of the one or more miRNAs in the biological sample to predict and/or determine responsiveness of the subject to the DHFR inhibitor, wherein the biological sample comprises cells with mutated RAS and no amplification of the RAS gene.

The present disclosure also provides methods for predicting and/or determining responsiveness of a subject with a disease or disorder to a dihydrofolate reductase (DHFR) inhibitor (e.g., an antifolate such as Methotrexate or Pemetrexed (ALTIMA™)), the method comprising: administering a DHFR inhibitor to the subject; obtaining a biological sample from the subject; assaying the biological sample for expression of one or more miRNAs; determining if expression of the one or more miRNAs in the biological sample is elevated or reduced compared to a control sample or above or below a threshold; and employing the determination of the expression of the one or more miRNAs in the biological sample to predict and/or determine responsiveness of the subject to the DHFR inhibitor, wherein the biological sample comprises cells with mutated RAS and no amplification of the RAS gene. The subject may be predicted or determined to be responsive to the DHFR inhibitor where expression of the one or more miRNAs (e.g., a member of the miR181 and/or miR143 families such as miR-181c or miR143) in the biological sample is elevated as compared to the control sample or is greater than the threshold. The threshold may be set at a level of miRNA expression in a control sample above which a subject is known to respond to treatment with the DHFR inhibitor and below which a subject is known to not respond to treatment with the DHFR inhibitor. In some embodiments, the threshold is set at a level of miRNA expression in the control sample above which 50%, 60%, 70%, 80%, 90%, or 95% of subject(s) respond to treatment with the DHFR inhibitor. In some embodiments, the threshold is set at a level of miRNA expression in the control sample below which 50%, 60%, 70%, 80%, 90%, or 95% of subject(s) do not respond to treatment with the DHFR inhibitor.

The present disclosure also provides methods for treating a subject with a disease or disorder, the method comprising: administering a DHFR inhibitor (e.g., an antifolate such as Methotrexate or Pemetrexed (ALTIMA™)) to the subject; assaying a biological sample from the subject for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and administering to the subject a therapeutically effective amount of DHFR inhibitor where the subject is predicted and/or determined to be responsive to the DHFR inhibitor, wherein the biological sample comprises cells with mutated RAS and no amplification of the RAS gene. Optionally, the methods may further comprise obtaining the biological sample. The subject may be predicted and/or determined to be responsive to the DHFR inhibitor where expression of the one or more miRNAs (e.g., a member of the miR181 and/or miR143 families such as miR-181c or miR143) in the biological sample is elevated as compared to the control sample or is greater than the threshold. The threshold may be set at a level of miRNA expression in a control sample above which a subject is known to respond to treatment with the DHFR inhibitor and below which a subject is known to not respond to treatment with the DHFR inhibitor. In some embodiments, the threshold is set at a level of miRNA expression in the control sample above which 50%, 60%, 70%, 80%, 90%, or 95% of subject(s) respond to treatment with the DHFR inhibitor. In some embodiments, the threshold is set at a level of miRNA expression in the control sample below which 50%, 60%, 70%, 80%, 90%, or 95% of subject(s) do not respond to treatment with the DHFR inhibitor.

The present disclosure also provides methods for selecting subjects for a clinical trial for testing the efficacy or safety of a dihydrofolate reductase (DHFR) inhibitor (e.g., an antifolate such as Methotrexate or Pemetrexed (ALTIMA™)), the method comprising: administering a DHFR inhibitor to the subject; obtaining a biological sample from the subjects; assaying the biological samples obtained from the subjects for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and selecting subjects for inclusion in a clinical trial that are predicted and/or determined to be responsive to the DHFR inhibitor, wherein the biological sample comprises one or more cells with mutated RAS and no amplification of the RAS gene. The subject may be predicted and/or determined to be responsive to the DHFR inhibitor where expression of the one or more miRNAs (e.g., a member of the miR181 and/or miR143 families such as miR-181c or miR143) in the biological sample is elevated as compared to the control sample or is greater than the threshold. The threshold may be set at a level of miRNA expression in a control sample above which a subject is known to respond to treatment with the DHFR inhibitor and below which a subject is known to not respond to treatment with the DHFR inhibitor. In some embodiments, the threshold is set at a level of miRNA expression in the control sample above which 50%, 60%, 70%, 80%, 90%, or 95% of subject(s) respond to treatment with the DHFR inhibitor. In some embodiments, the threshold is set at a level of miRNA expression in the control sample below which 50%, 60%, 70%, 80%, 90%, or 95% of subject(s) do not respond to treatment with the DHFR inhibitor.

Target cells may include, for example, cells to be treated (e.g., killed) by a drug. In some embodiments, target cells may include cancer cells.

In some embodiments, RAS mutation (e.g., mutated RAS) and/or RAS amplification may be detected in formalin-fixed paraffin-embedded (FFPE) tissue samples obtained from a subject.

A cell or biological sample may be considered responsive/sensitive to a drug if the dug induces apoptosis, and/or decreases cell proliferation of the cell and/or biological sample. Responsiveness of a cell or biological sample to a drug may also be measured as a reduction in size of the cell or biological sample. In some embodiments, the cell and/or biological sample may be considered responsive/sensitive to a drug where there is a greater than 50%, 60%, 70%, 80%, 90% or 95% likelihood that the cell and/or biological sample will be responsive/sensitive to the drug. In some embodiments, a cell or biological sample may be considered responsive/sensitive to a drug if the dug induces apoptosis, decreases cell proliferation of the cell and/or biological sample as compared to a control cell/control biological sample. Responsiveness of a cell or biological sample to a drug may also be measured as a reduction in size of the cell or biological sample as compared to the control cell or control biological sample. In some embodiments, the cell and/or biological sample may be considered responsive/sensitive to a drug where there is a greater than 50%, 60%, 70%, 80%, 90% or 95% likelihood that the cell and/or biological sample will be responsive/sensitive to the drug.

A subject including, for example, a human patient, may be predicted and/or determined responsive/sensitive to a drug if the dug induces apoptosis, decreases cell proliferation, or induces an immune response against a cell and/or biological sample obtained from the subject or patient. Responsiveness of the subject to a drug may also be measured as a reduction in size of the cell or biological sample.

A test cell may include a tumor cell. For examination of a long-term treatment effect, or effectiveness for individual patients, namely, tailor made medicine, it is possible to culture a cancer cell that can be obtained from a tumor of patient and use the cancer cell as a test cell.

In some embodiments, patients with a disease or disorder such as cancer or an autoimmune disease that are predicted to be responsive to a drug, including a chemotherapy such as Methotrexate, may be treated with an effective amount of the drug to treat the disease or disorder.

In some embodiments, “treating” or “treatment” of a disease, disorder, or condition includes at least partially: (1) preventing the disease, disorder, or condition, i.e. causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, i.e., arresting or reducing the development of the disease, disorder, or condition or its clinical symptoms; or (3) relieving the disease, disorder, or condition, i.e., causing regression of the disease, disorder, or condition or its clinical symptoms. The treating or treatment of a disease or disorder may include treating or the treatment of cancer.

The term “treatment of cancer” refers to administration to a mammal afflicted with a cancerous condition and refers to an effect that alleviates the cancerous condition by killing the cancerous cells, but also to an effect that results in the inhibition of growth and/or metastasis of the cancer.

An “effective amount,” as used herein, refers to the amount of an active composition that is required to confer a therapeutic effect on the subject. A “therapeutically effective amount,” as used herein, refers to a sufficient amount of an agent or a compound being administered which will relieve to some extent one or more of the symptoms of the disease, disorder, or condition being treated. In some embodiments, the result is a reduction and/or alleviation of the signs, symptoms, or causes of a disease, or any other desired alteration of a biological system. For example, in some embodiments, an “effective amount” for therapeutic uses is the amount of the composition including a compound as disclosed herein required to provide a clinically significant decrease in disease symptoms without undue adverse side effects. In some embodiments, an appropriate “effective amount” in any individual case is determined using techniques, such as a dose escalation study. The term “therapeutically effective amount” includes, for example, a prophylactically effective amount. In other embodiments, an “effective amount” of a compound disclosed herein, such as a compound of Formula (A) or Formula (I), is an amount effective to achieve a desired pharmacologic effect or therapeutic improvement without undue adverse side effects. In other embodiments, it is understood that “an effect amount” or “a therapeutically effective amount” varies from subject to subject, due to variation in metabolism, age, weight, general condition of the subject, the condition being treated, the severity of the condition being treated, and the judgment of the prescribing physician.

The term “chemotherapy” refers to the treatment of cancer or a disease or disorder caused by a virus, bacterium, other microorganism, or an inappropriate immune response using specific chemical agents, drugs, or radioactive agents that are selectively toxic and destructive to malignant cells and tissues, viruses, bacteria, or other microorganisms. Chemotherapeutic agents or drugs such as an anti-folate (e.g., Methotrexate) or any other agent or drug useful in treating cancer, an inflammatory disease, or an autoimmune disease are preferred. Suitable chemotherapeutic agents and drugs include, but are not limited to, actinomycin D, adriamycin, altretamine, azathioprine, bleomycin, busulphan, capecitabine, carboplatin, carmustine, chlorambucil, cisplatin, cladribine, crisantaspase, cyclophosphamide, cytarabine, dacarbazine, daunorubicin, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, Methotrexate, mitomycin, mitozantrone, oxaliplatin, paclitaxel, pentostatin, procarbazine, raltitrexed, steroids, streptozocin, taxol, taxotere, temozolomide, thioguanine, thiotepa, tomudex, topotecan, treosulfan, uft (uracil-tegufur), vinblastine, vincristine, vindesine, and vinorelbine. Methotrexate is especially preferred.

The term “Methotrexate” is synonymous with “MTX”. Methotrexate includes, in part, a 2,4-diamino substituted pterine ring moiety linked at the 6 position to the amino group of a p-aminobenzoyl moiety, the p-aminobenzoyl moiety having a methylated amino group and being amide bonded to a glutamic acid moiety.

The term “autoimmune disease” refers to a disease or disorder resulting from an immune response against a self tissue or tissue component and includes a self antibody response or cell-mediated response. The term autoimmune disease, as used herein, encompasses organ-specific autoimmune diseases, in which an autoimmune response is directed against a single tissue, such as Crohn's disease and ulcerative colitis, Type I diabetes mellitus, myasthenia gravis, vitiligo, Graves' disease, Hashimoto's disease, Addison's disease and autoimmune gastritis; and autoimmune hepatitis. The term autoimmune disease also encompasses non-organ specific autoimmune diseases, in which an autoimmune response is directed against a component present in several or many organs throughout the body. Such autoimmune diseases include, for example, rheumatoid disease, systemic lupus erythematosus, progressive systemic sclerosis and variants, polymyositis and dermatomyositis. Additional autoimmune diseases include, but are not limited to, pernicious anemia, autoimmune gastritis, primary biliary cirrhosis, autoimmune thrombocytopenia, Sjögren's syndrome, multiple sclerosis and psoriasis. One skilled in the art appreciates that the autoimmune diseases set forth above have been treated with chemotherapy such as Methotrexate therapy and further recognizes that the methods of the invention can be used to optimize clinical responsiveness to the chemotherapy in a human or other mammal having any of the above or another autoimmune disease.

In some embodiments, a RAS mutation may comprise one or more mutations of V-Ki-RAS2 Kirsten rat sarcoma viral oncogene homolog (KRAS) (SEQ ID NO: 1), NRAS (SEQ ID NO: 2) or HRAS (SEQ ID NO: 3). Alternatively, a RAS mutation may be a variant including, for example, a biologically active variant, of the amino acid sequence as set forth in SEQ ID NO: 1, 2, or 3.

In some embodiments a RAS amplification may comprise one or more amplifications of KRAS (SEQ ID NO: 4), NRAS (SEQ ID NO: 5) or HRAS (SEQ ID NO: 6). Alternatively, a RAS amplification may be a variant including, for example, a biologically active variant, of the nucleotide sequence as set forth in SEQ ID NO: 4, 5 or 6.

MiR-181 and/or miR143 family members may comprise the nucleotide sequence as set forth in SEQ ID NO: 7-13, respectively. Alternatively, miR-181 and/or miR143 family members may be a variant including, for example, a biologically active variant, of the nucleotide sequence as set forth in SEQ ID NO: 7-13, respectively.

Guidance in determining which nucleotides or amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

Protein variants include glycosylated forms, aggregative conjugates with other molecules, and covalent conjugates with unrelated chemical moieties. Also, protein variants also include allelic variants, species variants, and muteins. Truncations or deletions of regions which do not affect the differential expression of the gene are also variants. Covalent variants can be prepared by linking functionalities to groups which are found in the amino acid chain or at the N- or C-terminal residue, as is known in the art.

It will be recognized in the art that some amino acid sequence of RAS can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Thus, the polypeptides of the present invention may include one or more amino acid substitutions, deletions or additions, either from natural mutations or human manipulation.

Amino acids in the polypeptides of the present invention that are essential for function can be identified by methods known in the art, such as site-directed mutagenesis or alanine-scanning mutagenesis (Cunningham and Wells, Science 244: 1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith, et al., J. Mol. Biol. 224:899-904 (1992) and de Vos, et al. Science 255:306-312 (1992)).

Variants of the RAS gene may include a polynucleotide possessing a nucleotide sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to RAS. Variants of RAS may include a polypeptide possessing an amino acid sequence that possess at least 90% sequence identity, more preferably at least 91% sequence identity, even more preferably at least 92% sequence identity, still more preferably at least 93% sequence identity, still more preferably at least 94% sequence identity, even more preferably at least 95% sequence identity, still more preferably at least 96% sequence identity, even more preferably at least 97% sequence identity, still more preferably at least 98% sequence identity, and most preferably at least 99% sequence identity, to RAS. Preferably, this variant may possess at least one biological property in common with the native protein.

Sequence identity or percent identity is intended to mean the percentage of the same residues shared between two sequences, when the two sequences are aligned using the Clustal method [Higgins et al, Cabios 8:189-191 (1992)] of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=10; gap length penalty for multiple alignment=10; k-tuple value in pairwise alignment=1; gap penalty in pairwise alignment=3; window value in pairwise alignment=5; diagonals saved in pairwise alignment=5. The residue weight table used for the alignment program is PAM250 [Dayhoff, et al., in Atlas of Protein Sequence and Structure, Dayhoff, Ed., NDRF, Washington, Vol. 5, suppl. 3, p. 345, (1978)].

In one embodiment, the disease or disorder may be cancer. In one embodiment the cancer may be selected from the group consisting of: oral cancer, prostate cancer, rectal cancer, non-small cell lung cancer, lip and oral cavity cancer, liver cancer, lung cancer, anal cancer, kidney cancer, vulvar cancer, breast cancer, oropharyngeal cancer, nasal cavity and paranasal sinus cancer, nasopharyngeal cancer, urethra cancer, small intestine cancer, bile duct cancer, bladder cancer, ovarian cancer, laryngeal cancer, hypopharyngeal cancer, gallbladder cancer, colon cancer, colorectal cancer, head and neck cancer, glioma; parathyroid cancer, penile cancer, vaginal cancer, thyroid cancer, pancreatic cancer, esophageal cancer, Hodgkin's lymphoma, leukemia-related disorders, mycosis fungoides, and myelodysplastic syndrome.

In another embodiment the cancer may be non-small cell lung cancer, pancreatic cancer, breast cancer, ovarian cancer, colorectal cancer, or head and neck cancer. In yet another embodiment the cancer may be a carcinoma, a tumor, a neoplasm, a lymphoma, a melanoma, a glioma, a sarcoma, or a blastoma.

In one embodiment the carcinoma may be selected from the group consisting of: carcinoma, adenocarcinoma, adenoid cystic carcinoma, adenosquamous carcinoma, adrenocortical carcinoma, well differentiated carcinoma, squamous cell carcinoma, serous carcinoma, small cell carcinoma, invasive squamous cell carcinoma, large cell carcinoma, islet cell carcinoma, oat cell carcinoma, squamous carcinoma, undifferentiated carcinoma, verrucous carcinoma, renal cell carcinoma, papillary serous adenocarcinoma, merkel cell carcinoma, hepatocellular carcinoma, soft tissue carcinomas, bronchial gland carcinomas, capillary carcinoma, bartholin gland carcinoma, basal cell carcinoma, carcinosarcoma, papilloma/carcinoma, clear cell carcinoma, endometrioid adenocarcinoma, mesothelial, metastatic carcinoma, mucoepidermoid carcinoma, cholangiocarcinoma, actinic keratoses, cystadenoma, and hepatic adenomatosis.

In another embodiment the tumor may be selected from the group consisting of: astrocytic tumors, malignant mesothelial tumors, ovarian germ cell tumors, supratentorial primitive neuroectodermal tumors, Wilms tumors, pituitary tumors, extragonadal germ cell tumors, gastrinoma, germ cell tumors, gestational trophoblastic tumors, brain tumors, pineal and supratentorial primitive neuroectodermal tumors, pituitary tumors, somatostatin-secreting tumors, endodermal sinus tumors, carcinoids, central cerebral astrocytoma, glucagonoma, hepatic adenoma, insulinoma, medulloepithelioma, plasmacytoma, vipoma, and pheochromocytoma.

In yet another embodiment the neoplasm may be selected from the group consisting of: intraepithelial neoplasia, multiple myeloma/plasma cell neoplasm, plasma cell neoplasm, interepithelial squamous cell neoplasia, endometrial hyperplasia, focal nodular hyperplasia, hemangioendothelioma, and malignant thymoma. In a further embodiment the lymphoma may be selected from the group consisting of: nervous system lymphoma, AIDS-related lymphoma, cutaneous T-cell lymphoma, non-Hodgkin's lymphoma, lymphoma, and Waldenstrom's macroglobulinemia. In another embodiment the melanoma may be selected from the group consisting of: acral lentiginous melanoma, superficial spreading melanoma, uveal melanoma, lentigo maligna melanomas, melanoma, intraocular melanoma, adenocarcinoma nodular melanoma, and hemangioma. In yet another embodiment the sarcoma may be selected from the group consisting of: adenomas, adenosarcoma, chondosarcoma, endometrial stromal sarcoma, Ewing's sarcoma, Kaposi's sarcoma, leiomyosarcoma, rhabdomyosarcoma, sarcoma, uterine sarcoma, osteosarcoma, and pseudosarcoma. In one embodiment the glioma may be selected from the group consisting of: glioma, brain stem glioma, and hypothalamic and visual pathway glioma. In another embodiment the blastoma may be selected from the group consisting of: pulmonary blastoma, pleuropulmonary blastoma, retinoblastoma, neuroblastoma, medulloblastoma, glioblastoma, and hemangiblastomas.

Biological samples or test cells that may be used in the methods of the present disclosure may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, serum, lymph fluid, urine and faeces, or tissue which has been removed from organs, such as breast, lung, intestine, skin, cervix, prostate, and stomach. Biological samples may also include sections of tissues such as frozen sections taken for histological purposes.

### Antifolates

Antifolates such as Methotrexate and Pemetrexed are antineoplastic agents that affect the folic acid pathway and have been used to treat many solid tumors and hematologic malignancies. Folic acid is essential for the synthesis of DNA and RNA precursors and for the synthesis of thymidine nucleotide that is incorporated exclusively into DNA. Folic acid in its fully reduced form serves as a one-carbon carrier required for the synthesis of thymidylate, purine nucleotides, and certain amino acids. Normal cells, as well as tumor cells, use the pool of active reduced folate to proliferate. The cytotoxic activity of antifolates in particular is mainly due to their ability to inhibit several different folate-dependent enzymes involved in DNA synthesis.

Methotrexate is well known in the art as an inhibitor of dihydrofolate reductase (DHFR), which acts to decrease production of tetrahydrofolate (THF) from dihydrofolate (DHF). As a consequence, Methotrexate indirectly inhibits purine and thymidine synthesis and amino acid interconversion. Methotrexate also exhibits anti-proliferative activity through inhibition of thymidylate synthesis, which is required to synthesize DNA (Calvert, Semin. Oncol. 26:3-10 (1999)). Methotrexate, its synthesis, and its properties are described in further detail in U.S. Pat. Nos. 2,512,572; 3,892,801; 3,989,703; 4,057,548; 4,067,867; 4,079,056; 4,080,325; 4,136,101; 4,224,446; 4,306,064; 4,374,987; 4,421,913; and 4,767,859. Methods of using Methotrexate to treat cancer are described, for example, in U.S. Pat. Nos. 4,106,488, 4,558,690, and 4,662,359.

Methotrexate, which is useful in the treatment of a variety of autoimmune diseases and cancers, can be administered by oral or parenteral routes. The drug is readily distributed to body tissues, where it is transported into cells by a specific carrier system that includes components such as the reduced folate carrier, RFC-1, and the folate receptor. Due to its high polarity at physiological pH, Methotrexate does not readily pass through the cell membrane, and the majority of the drug enters cells via specific carriers. Once inside the cell, Methotrexate is converted to Methotrexate polyglutamates by specific enzymes such as folylpolygamma-glutamate synthetase, which add one or more glutamic acid moieties, linked by iso-peptidic bonds to the γ-carboxyl of Methotrexate as described, for example, in Kamen, Semin. Oncol. S18:30-39 (1997).

Pemetrexed (e.g., Alimta®, Eli Lilly and Co., Indianapolis, Ind.) is an antifolate agent that exerts its action by disrupting folate-dependent metabolic processes essential for cell replication. In February 2004, the United States Food and Drug Administration (FDA) approved Pemetrexed for the treatment of MPM and 6 months later for non-small cell lung cancer (NSCLC). Pemetrexed acts as a multi-targeted antifolate that strongly inhibit various folate-dependent enzymes, including thymidylate synthase (TS), dihydrofolate reductase (DHFR), glycinamide ribonucleotide formyltransfeRASe (GARFT) and the like, and have excellent anti-tumor activities.

Detection of miRNA Expression

MiRNA (e.g., expression of one or more of miR-143 and/or miR-181c) may be assayed (e.g., determined) by methods which detect particular mRNAs in cells. These include, hybridization assays using complementary DNA probes (such as in situ hybridization using labeled riboprobes, Northern blot and related techniques) and various nucleic acid amplification assays (such as RT-PCR using complementary primers specific for miR-143 and/or miR-181c, and other amplification type detection methods, such as, for example, branched DNA, SISBA, TMA and the like). Protocols for the detection of specific mRNAs in a sample are well known in the art (Sambrook et al., (1990) Molecular Cloning—A Laboratory Manual, Cold Spring Harbor Laboratory Press; Ausubel et al., (1998) Current Protocols in Molecular Biology, Wiley).

MiRNAs that may be detected by the methods of the instant disclosure include those miRNAs from the miR-181 family and from the miR-143 family as referenced in Table 1 below.

TABLE 1
MiRNA sequences

| MiRNA | | | | SEQ ID |
|---|---|---|---|---|
| family | Synonym | Accession # | Sequence | NO |
| miR- | hsa-mir181a-1 | M10000289 | UGAGUUUUGAGGUUGCUUCAGUGAACAUU | 7 |
| 181 | | | CAACGCUGUCGGUGAGUUUGGAAUUAAAA | |
| | | | UCAAAACCAUCGACCGUUGAUUGUACCCU | |
| | | | AUGGCUAACCAUCAUCUACUCCA | |
| | hsa-miR181-a2 | M10000269 | AGAAGGGCUAUCAGGCCAGCCUUCAGAGG | 8 |
| | | | ACUCCAAGGAACAUUCAACGCUGUCGGUG | |
| | | | AGUUUGGGAUUUGAAAAAACCACUGACCG | |
| | | | UUGACUGUACCUUGGGGUCCUUA | |
| | hsa-miR181-b1 | M10000270 | CCUGUGCAGAGAUUAUUUUUUAAAAGGUC | 9 |
| | | | ACAAUCAACAUUCAUUGCUGUCGGUGGGU | |
| | | | UGAACUGUGUGGACAAGCUCACUGAACAA | |
| | | | UGAAUGCAACUGUGGCCCCGCUU | |
| | hsa-miR181-b2 | M10000683 | CUGAUGGCUGCACUCAACAUUCAUUGCUG | 10 |
| | | | UCGGUGGGUUUGAGUCUGAAUCAACUCAC | |
| | | | UGAUCAAUGAAUGCAAACUGCGGACCAAA | |
| | | | CA | |
| | hsa-miR181-c | M10000271 | CGGAAAAUUUGCCAAGGGUUUGGGGGAAC | 11 |
| | | | AUUCAACCUGUCGGUGAGUUUGGGCAGCU | |
| | | | CAGGCAAACCAUCGACCGUUGAGUGGACC | |
| | | | CUGAGGCCUGGAAUUGCCAUCCU | |
| | hsa-miR181-d | M10003139 | GUCCCCUCCCCUAGGCCACAGCCGAGGUC | 12 |
| | | | ACAAUCAACAUUCAUUGUUGUCGGUGGGU | |
| | | | UGUGAGGACUGAGGCCAGACCCACCGGGG | |
| | | | GAUGAAUGUCACUGUGGCUGGGCCAGACA | |
| | | | CGGCUUAAGGGGAAUGGGGAC | |
| miR- | hsa-miR143 | M10000459 | GCGCAGCGCCCUGUCUCCCAGCCUGAGGU | 13 |
| 143 | | | GCAGUGCUGCAUCUCUGGUCAGUUGGGAG | |
| | | | UCUGAGAUGAAGCACUGUAGCUCAGGAAG | |
| | | | AGAGAAGUUGUUCUGCAGC | |

Detection of the RNA products of the molecular marker genes may be accomplished by a variety of methods. Some methods are quantitative and allow estimation of the original levels of RNA between the cancer and control cells, whereas other methods are merely qualitative. Additional information regarding the methods presented below may be found in Ausubel et al., (2003) Current Protocols in Molecular Biology, John Wiley & Sons, New York, N.Y., or Sambrook et al. (1989) Molecular Cloning: A Laboratory Manual, Cold Spring Harbor Press, Cold Spring Harbor, N.Y. A person skilled in the art will know which parameters may be manipulated to optimize detection of the mRNA of interest.

Quantitative real-time PCR (QRT-PCR) may be used to measure the differential expression of a molecular marker in a test cell and a control cell. In QRT-PCR, the RNA template is generally reverse transcribed into cDNA, which is then amplified via a PCR reaction. The PCR amplification process is catalyzed by a thermostable DNA polymeRASe. Non-limiting examples of suitable thermostable DNA polymeRASes include Taq DNA polymeRASe, Pfu DNA polymeRASe, Tli (also known as Vent) DNA polymeRASe, Tfl DNA polymeRASe, and Tth DNA polymeRASe. The PCR process may comprise 3 steps (i.e., denaturation, annealing, and extension) or 2 steps (i.e., denaturation and annealing/extension). The temperature of the annealing or annealing/extension step can and will vary, depending upon the amplification primers. That is, their nucleotide sequences, melting temperatures, and/or concentrations. The temperature of the annealing or annealing/extending step may range from about 50° C. to about 75° C. The amount of PCR product is followed cycle-by-cycle in real time, which allows for determination of the initial concentrations of mRNA. The reaction may be performed in the presence of a dye that binds to double-stranded DNA, such as SYBR Green. The reaction may also be performed with a fluorescent reporter probes, such as TAQMAN® probes (Applied Biosystems, Foster City, Calif.) that fluoresce when the quencher is removed during the PCR extension cycle. Fluorescence values are recorded during each cycle and represent the amount of product amplified to that point in the amplification reaction. The cycle when the fluorescent signal is first recorded as statistically significant is the threshold cycle (Ct). To minimize errors and reduce any sample-to-sample variation, QRT-PCR is typically performed using an internal standard. The ideal internal standard is expressed at a constant level among different tissues, and is unaffected by the experimental treatment. Suitable internal standards include, but are not limited to, mRNAs for the housekeeping genes glyceraldehyde-3-phosphate-dehydrogenase (GAPDH) and beta-actin.

Reverse-transcriptase PCR (RT-PCR) may also be used to measure the differential expression of a molecular marker. As above, the RNA template is reverse transcribed into cDNA, which is then amplified via a typical PCR reaction. After a set number of cycles the amplified DNA products are typically separated by gel electrophoresis. Comparison of the relative amount of PCR product amplified in the different cells will reveal whether the molecular marker is differentially expressed in the cancer cell.

Differential expression of a molecular marker may also be measured using a nucleic acid microarray. In this method, single-stranded nucleic acids (e.g., cDNAs, oligonucleotides, etc.) are plated, or arrayed, on a solid support. The solid support may be a material such as glass, silica-based, silicon-based, a synthetic polymer, a biological polymer, a copolymer, a metal, or a membrane. The form or shape of the solid support may vary, depending on the application. Suitable examples include, but are not limited to, slides, strips, plates, wells, microparticles, fibers (such as optical fibers), gels, and combinations thereof. The arrayed immobilized sequences are generally hybridized with specific DNA probes from the cells of interest. Fluorescently labeled cDNA probes may be generated through incorporation of fluorescently labeled deoxynucleotides by reverse transcription of RNA extracted from the cells of interest. The probes are hybridized to the immobilized nucleic acids on the microchip under highly stringent conditions. After stringent washing to remove non-specifically bound probes, the chip is scanned by confocal laser microscopy or by another detection method, such as a CCD camera. Quantitation of hybridization of each arrayed element allows for assessment of corresponding mRNA abundance. With dual color fluorescence, separately labeled cDNA probes generated from two sources of RNA are hybridized pairwise to the array. The relative abundance of the transcripts from the two sources corresponding to each specified molecular marker is thus determined simultaneously. Microarray analysis may be performed by commercially available equipment, following manufacturer's protocols, such as by using the Affymetrix GenChip technology, or Incyte's microarray technology.

Differential expression of a molecular marker may also be measured using Northern blotting. For this, RNA samples are first separated by size via electrophoresis in an agarose gel under denaturing conditions. The RNA is then transferred to a membrane, crosslinked, and hybridized, under highly stringent conditions, to a labeled DNA probe. After washing to remove the non-specifically bound probe, the hybridized labeled species are detected using techniques well known in the art. The probe may be labeled with a radioactive element, a chemical that fluoresce when exposed to ultraviolet light, a tag that is detected with an antibody, or an enzyme that catalyses the formation of a colored or a fluorescent product. A comparison of the relative amounts of RNA detected in the different cells will reveal whether the expression of the molecular marker is changed in the cancer cell.

Nuclease protection assays may also be used to monitor the differential expression of a molecular marker in cancer and control cells. In nuclease protection assays, an antisense probe hybridizes in solution to an RNA sample. The antisense probe may be labeled with an isotope, a fluorophore, an enzyme, or another tag. Following hybridization, nucleases are added to degrade the single-stranded, unhybridized probe and RNA. An acrylamide gel is used to separate the remaining protected double-stranded fragments, which are then detected using techniques well known in the art. Again, qualitative differences in expression may be detected.

Differential expression of a molecular marker may also be measured using in situ hybridization. This type of hybridization uses a labeled antisense probe to localize a particular mRNA in cells of a tissue section. The hybridization and washing steps are generally performed under highly stringent conditions. The probe may be labeled with a fluorophore or a small tag (such as biotin or digoxigenin) that may be detected by another protein or antibody, such that the labeled hybrid may be visualized under a microscope. The transcripts of a molecular marker may be localized to the nucleus, the cytoplasm, or the plasma membrane of a cell.

Expression of the molecular marker or markers will generally be measured in a cancer cell relative to a control cell. The cell may be isolated from a subject so that expression of the marker may be examined in vitro. The type of biopsy used to isolated cells can and will vary, depending upon the location and nature of the cancer.

A sample of cells, tissue, or fluid may be removed by needle aspiration biopsy. For this, a fine needle attached to a syringe is inserted through the skin and into the organ or tissue of interest. The needle is typically guided to the region of interest using ultRASound or computed tomography (CT) imaging. Once the needle is inserted into the tissue, a vacuum is created with the syringe such that cells or fluid may be sucked through the needle and collected in the syringe. A sample of cells or tissue may also be removed by incisional or core biopsy. For this, a cone, a cylinder, or a tiny bit of tissue is removed from the region of interest. This type of biopsy is generally guided by CT imaging, ultRASound, or an endoscope. Lastly, the entire cancerous tumor may be removed by excisional biopsy or surgical resection.

RNA, protein, or DNA may be extracted from the biopsied cells or tissue to permit analysis of the expression of a molecular marker using methods described above in section (I)(d). The biopsied cells or tissue may also be embedded in plastic or paraffin, from which nucleic acids may be isolated. The expression of a molecular marker may also be performed in the biopsied cells or tissue in situ (e.g., in situ hybridization, immunohistochemistry).

Expression of a molecular marker may also be examined in vivo in a subject. A particular mRNA or protein may be labeled with fluorescent dye, a bioluminescent marker, a fluorescent semiconductor nanocrystal, or a short-lived radioisotope, and then the subject may be imaged or scanned using a variety of techniques, depending upon the type of label.

Detection of RAS Mutation and/or Amplification

A number of methodologies may be employed to detect the presence or absence including quantitating the expression (i.e., expression level or amount) of mutated RAS (e.g., one or more mutations in KRAS, (SEQ ID NO: 1), NRAS (SEQ ID NO: 2), or HRAS (SEQ ID NO: 3) and/or the presence or absence of an amplification (e.g., 3, 4, 5, 6, 7, 8, 9, 10, 11, 12, 13, 14, 15, 16, 17, 18, 19, 20 or more copies per cell) of a RAS gene including, KRAS, (SEQ ID NO: 4), NRAS (SEQ ID NO: 5), or HRAS (SEQ ID NO: 6) in a cell and/or a biological sample. Such detection of mutated RAS and/or amplification of RAS may be detected at the protein level and/or nucleic acid level. Those skilled in the art will appreciate that the methods indicated below represent some of the preferred ways in which the presence or absence, including the expression, of mutated RAS and/or the presence or absence of a RAS amplification may be detected and/or quantitated and in no manner limit the scope of methodologies that may be employed. Those skilled in the art will also be able to determine operative and optimal assay conditions for each determination by employing routine experimentation. Such methods may include but are not limited to in situ hybridization (ISH), Western blots, ELISA, immunoprecipitation, immunofluorescence, flow cytometry, northern blots, PCR, and immunocytochemistry (IHC). RAS may comprise the amino acid sequence set forth in SEQ ID NOS 1, 2 or 3. Alternatively, RAS may be a variant of the amino acid sequence as set forth in SEQ ID NOS 1, 2 or 3. A RAS amplification may comprise one or more amplifications of KRAS (SEQ ID NO: 4), NRAS (SEQ ID NO: 5) or HRAS (SEQ ID NO: 6). Alternatively, a RAS amplification may be a variant including, for example, a biologically active variant, of the nucleotide sequence as set forth in SEQ ID NO: 4, 5 or 6.

In another embodiment, the methods may further involve obtaining a control sample and detecting mutated RAS and/or amplification of RAS in this control sample, such that the presence or absence mutated RAS and/or amplification of RAS in the control sample is determined. A negative control sample is useful if there is an absence of mutated RAS and/or amplification of RAS, whereas a positive control sample is useful if there is a presence of mutated RAS and/or amplification of RAS. For the negative control, the sample may be from the same individual as the test sample (i.e. different location such as tumor versus non-tumor) or may be from a different individual known to have an absence of mutated RAS and/or amplification of RAS.

A biological sample may include tissues, cells, biological fluids and isolates thereof, isolated from a subject, as well as tissues, cells and fluids present within a subject (e.g., a patient). Preferably, biological samples comprise cells, most preferably tumor cells, that are isolated from body samples, such as, but not limited to, smears, sputum, biopsies, secretions, cerebrospinal fluid, bile, blood, lymph fluid, urine and faeces, or tissue which has been removed from organs, such as breast, lung, intestine, skin, cervix, prostate, and stomach.

### Detection/Quantitation of RAS Mutation

In an embodiment, mutated RAS may be detected at the nucleic acid or protein level. Nucleic acid-based techniques for assessing expression are well known in the art and include, for example, determining the level of RAS mRNA in a biological sample. Many expression detection methods use isolated RNA. Any RNA isolation technique that does not select against the isolation of mRNA can be utilized for the purification of RNA from cervical cells (see, e.g., Ausubel et al., ed., (1987-1999) Current Protocols in Molecular Biology (John Wiley & Sons, New York). Additionally, large numbers of tissue samples can readily be processed using techniques well known to those of skill in the art, such as, for example, the single-step RNA isolation process of Chomczynski (1989, U.S. Pat. No. 4,843,155).

Isolated mRNA from a biological sample can be used in hybridization or amplification assays that include, but are not limited to, Southern or Northern analyses, polymeRASe chain reaction analyses and probe arrays. One method for the detection of RAS mRNA levels involves contacting the isolated mRNA with a nucleic acid molecule (probe) that can hybridize to the mRNA encoded by the RAS gene. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to an mRNA or genomic DNA encoding RAS. Hybridization of an mRNA with the probe indicates that RAS is being expressed.

In one embodiment, the mRNA from a biological sample is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array.

An alternative method for determining the level of RAS mRNA in a biological sample involves the process of nucleic acid amplification, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In particular aspects of the disclosure, biomarker expression may be assessed by quantitative fluorogenic RT-PCR (i.e., the TaqMan® System). Such methods typically may utilize pairs of oligonucleotide primers that are specific for RAS. Methods for designing oligonucleotide primers specific for a known sequence are well known in the art.

Expression levels of RAS RNA may be monitored using a membrane blot (such as used in hybridization analysis such as Northern, Southern, dot, and the like), or microwells, sample tubes, gels, beads or fibers (or any solid support comprising bound nucleic acids) (see, e.g., U.S. Pat. Nos. 5,770,722, 5,874,219, 5,744,305, 5,677,195 and 5,445,934). The detection of RAS expression may also comprise using nucleic acid probes in solution.

In one embodiment of the disclosure, microarrays are used to detect RAS expression. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA may be hybridized to complementary probes on the array and then detected by laser scanning. Hybridization intensities for each probe on the array are determined and converted to a quantitative value representing relative gene expression levels (see, e.g., U.S. Pat. Nos. 6,040,138, 5,800,992, 6,020,135, 6,033,860, and 6,344,316). High-density oligonucleotide arrays are particularly useful for determining the gene expression profile for a large number of RNA's in a sample.

Techniques for the synthesis of these arrays using mechanical synthesis methods are described in, e.g., U.S. Pat. No. 5,384,261. Although a planar array surface is preferred, the array may be fabricated on a surface of virtually any shape or even a multiplicity of surfaces. Arrays may be peptides or nucleic acids on beads, gels, polymeric surfaces, fibers such as fiber optics, glass or any other appropriate substrate, see U.S. Pat. Nos. 5,770,358, 5,789,162, 5,708,153, 6,040,193 and 5,800,992. Arrays may be packaged in such a manner as to allow for diagnostics or other manipulation of an all-inclusive device (see, e.g., U.S. Pat. Nos. 5,856,174 and 5,922,591).

In one approach, total mRNA isolated from the biological sample may be converted to labeled cRNA and then hybridized to an oligonucleotide array. Each sample may be hybridized to a separate array. Relative transcript levels may be calculated by reference to appropriate controls present on the array and in the sample.

In a particular embodiment, the level of RAS mRNA can be determined both by in situ and by in vitro formats in a biological sample using methods known in the art. Many expression detection methods use isolated RNA. For in vitro methods, any RNA isolation technique that does not select against the isolation of mRNA can be utilized for the purification of RNA from tumor cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (see, e.g., U.S. Pat. No. 4,843,155).

The isolated mRNA can be used in hybridization or amplification assays that include, but are not limited to, Southern or Northern analyses, polymeRASe chain reaction analyses and probe arrays. One preferred diagnostic method for the detection of RAS mRNA levels involves contacting the isolated mRNA with a nucleic acid molecule (probe) that can hybridize to the RAS mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding RAS. Other suitable probes for use in the diagnostic assays of the disclosure are described herein. Hybridization of an mRNA with the probe indicates that RAS is being expressed.

In one format, the mRNA may be immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA may be contacted with the probe(s), for example, in an Affymetrix gene chip array. A skilled artisan can readily adapt known mRNA detection methods for use in detecting the level of mRNA encoded by RAS.

An alternative method for determining the level of RAS mRNA in a biological sample involves the process of nucleic acid amplification, e.g., by RT-PCR (see, e.g., U.S. Pat. No. 4,683,202), ligase chain reaction (Barany, 1991, Proc. Natl. Acad. Sci. USA, 88:189-193), self sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the tumor cells prior to detection. In such methods, a cell or tissue sample may be prepared/processed using known histological methods. The sample may be then immobilized on a support, typically a glass slide, and then contacted with a probe that can hybridize to RAS mRNA.

In another embodiment of the present disclosure, a RAS protein may be detected. A preferred agent for detecting RAS protein of the disclosure is an antibody capable of binding to such a protein or a fragment thereof, preferably an antibody with a detectable label. Antibodies can be polyclonal, or more preferably, monoclonal. An intact antibody, or a fragment or derivative thereof can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that may be directly labeled. Examples of indirect labeling include detection of a primary antibody using a fluorescently labeled secondary antibody and end-labeling of a DNA probe with biotin such that it can be detected with fluorescently labeled streptavidin.

Antibody fragments may comprise a portion of an intact antibody, preferably the antigen-binding or variable region of the intact antibody. Examples of antibody fragments include Fab, Fab′, F(ab′)2, and Fv fragments; diabodies; linear antibodies (Zapata et al. (1995) Protein Eng. 8(10):1057-1062); single-chain antibody molecules; and multispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize 35 readily. Pepsin treatment yields an F(ab′)2 fragment that has two antigen-combining sites and may be still capable of cross-linking antigen.

Detection of antibody binding can be facilitated by coupling the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesteRASe; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include lucifeRASe, luciferin, and aequorin; and examples of suitable radioactive material include ¹²⁵I, ¹³¹I, ³⁵S, or ³H.

In regard to detection of antibody staining in the immunocytochemistry methods of the disclosure, there also exist in the art, video-microscopy and software methods for the quantitative determination of an amount of multiple molecular species (e.g., biomarker proteins) in a biological sample wherein each molecular species present may be indicated by a representative dye marker having a specific color. Such methods are also known in the art as a colorimetric analysis methods. In these methods, video-microscopy may be used to provide an image of the biological sample after it has been stained to visually indicate the presence of a particular biomarker of interest. Some of these methods, such as those disclosed in U.S. patent application Ser. Nos. 09/957,446 and 10/057,729, disclose the use of an imaging system and associated software to determine the relative amounts of each molecular species present based on the presence of representative color dye markers as indicated by those color dye markers' optical density or transmittance value, respectively, as determined by an imaging system and associated software. These techniques provide quantitative determinations of the relative amounts of each molecular species in a stained biological sample using a single video image that may be deconstructed into its component color parts.

The antibodies used to practice the disclosure are selected to have high specificity for RAS including, for example, mutated RAS. Methods for making antibodies and for selecting appropriate antibodies are known in the art (see, e.g., Celis, ed. (in press) Cell Biology & Laboratory Handbook, 3rd edition (Academic Press, New York)). In some embodiments, commercial antibodies directed to specific RAS proteins may be used to practice the disclosure. The antibodies of the disclosure may be selected on the basis of desirable staining of cytological, rather than histological, samples. That is, in particular embodiments the antibodies are selected with the end sample type (i.e., cytology preparations) in mind and for binding specificity.

One of skill in the art will recognize that optimization of antibody titer and detection chemistry may be needed to maximize the signal to noise ratio for a particular antibody. Antibody concentrations that maximize specific binding to RAS and minimize non-specific binding (or background) can be determined. In particular embodiments, appropriate antibody titers for use in cytology preparations are determined by initially testing various antibody dilutions on formalin-fixed paraffin-embedded normal and high-grade cervical disease tissue samples. Optimal antibody concentrations and detection chemistry conditions are first determined for formalin-fixed paraffin-embedded tissue samples. The design of assays to optimize antibody titer and detection conditions is standard and well within the routine capabilities of those of ordinary skill in the art. After the optimal conditions for fixed tissue samples are determined, each antibody may be then used in cytology preparations under the same conditions. Some antibodies require additional optimization to reduce background staining and/or to increase specificity and sensitivity of staining in the cytology samples.

Furthermore, one of skill in the art will recognize that the concentration of a particular antibody used to practice the methods of the disclosure will vary depending on such factors as time for binding, level of specificity of the antibody for RAS protein, and method of body sample preparation. Moreover, when multiple antibodies are used, the required concentration may be affected by the order in which the antibodies are applied to the sample, i.e., simultaneously as a cocktail or sequentially as individual antibody reagents. Furthermore, the detection chemistry used to visualize antibody binding to a biomarker of interest must also be optimized to produce the desired signal to noise ratio.

Proteins from tumor cells can be isolated using techniques that are well known to those of skill in the art. The protein isolation methods employed can, for example, be such as those described in Harlow and Lane (Harlow and Lane, 1988, Antibodies: A Laboratory Manual, Cold Spring Harbor Laboratory Press, Cold Spring Harbor, N.Y.).

A variety of formats can be employed to determine whether a sample contains a protein that binds to a given antibody. Examples of such formats include, but are not limited to, enzyme immunoassay (EIA), radioimmunoassay (RIA), Western blot analysis and enzyme linked immunoabsorbant assay (ELISA). A skilled artisan can readily adapt known protein/antibody detection methods for use in determining whether tumor cells express a biomarker of the present disclosure.

One skilled in the art will know many other suitable carriers for binding antibody or antigen, and will be able to adapt such support for use with the present disclosure. For example, protein isolated from tumor cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means.

For ELISA assays, specific binding pairs can be of the immune or non-immune type. Immune specific binding pairs are exemplified by antigen-antibody systems or hapten/anti-hapten systems. There can be mentioned fluorescein/anti-fluorescein, dinitrophenyl/anti-dinitrophenyl, biotin/anti-biotin, peptide/anti-peptide and the like. The antibody member of the specific binding pair can be produced by customary methods familiar to those skilled in the art. Such methods involve immunizing an animal with the antigen member of the specific binding pair. If the antigen member of the specific binding pair is not immunogenic, e.g., a hapten, it can be covalently coupled to a carrier protein to render it immunogenic. Non-immune binding pairs include systems wherein the two components share a natural affinity for each other but are not antibodies.

The present disclosure also includes methods for fixing cells and tissue samples for analysis. Generally, neutral buffered formalin may be used. Any concentration of neutral buffered formalin that can fix tissue or cell samples without disrupting the epitope can be used. In one embodiment a solution of about 10 percent may be used. Preferably, the method includes suitable amounts of phosphatase inhibitors to inhibit the action of phosphatases and preserve phosphorylation. Any suitable concentration of phosphatase inhibitor can be used so long as the biopsy sample is stable and phosphatases are inhibited, for example 1 mM NaF and/or Na3VO4 can be used. In one method a tissue sample or tumor biopsy may be removed from a patient and immediately immersed in a fixative solution which can and preferably does contain one or more phosphatase inhibitors, such as NaF and/or Na3VO4. Preferably, when sodium orthovanadate is used it is used in an activated or depolymerized form to optimize its activity.

Depolymerization can be accomplished by raising the pH of its solution to about 10 and boiling for about 10 minutes. The phosphatase inhibitors can be dissolved in the fixative just prior to use in order to preserve their activity.

Fixed samples can then be stored for several days or processed immediately. To process the samples into paraffin after fixing, the fixative can be thoroughly rinsed away from the cells by flushing the tissue with water. The sample can be processed to paraffin according to normal histology protocols which can include the use of reagent grade ethanol. Samples can be stored in 70% ethanol until processed into paraffin blocks. Once samples are processed into paraffin blocks they can be analyzed histochemically for virtually any antigen that is stable to the fixing process.

In preferred embodiments, RAS staining may be detected, measured and quantitated automatically using automated image analysis equipment. Such equipment can include a light or fluorescence microscope, and image-transmitting camera and a view screen, most preferably also comprising a computer that can be used to direct the operation of the device and store and manipulate the information collected, most preferably in the form of optical density of certain regions of a stained tissue preparation. Image analysis devices useful in the practice of this disclosure include but are not limited to the CAS 200 (Becton Dickenson, Mountain View, Calif.), Chromavision or Tripath systems. Using such equipment the quantity of the target epitope in unknown cell samples can be determined using any of a variety of methods that are known in the art. The cell pellets can be analyzed by eye such that the optical density reading of the control cells can be correlated to a manual score such as 0, 1+, 2+ or 3+, as in Table 1 below which shows the correlation between quantitative image analysis data measured in optical density (OD) and manual score.

Automated (computer-aided) image analysis systems known in the art can augment visual examination of biological samples. In a representative system, the cell or tissue sample may be exposed to detectably labeled reagents specific for RAS (e.g., mutated RAS), and the magnified image of the cell may be then processed by a computer that receives the image from a charge-coupled device (CCD) or camera such as a television camera. Such a system can be used, for example, to detect and measure expression and activation levels of Her1, pHER1 HER2, HER3, and pERK in a sample. Additional biomarkers are also contemplated by this disclosure. This methodology provides more accurate diagnosis of cancer and a better characterization of gene expression in histologically identified cancer cells, most particularly with regard to expression of tumor marker genes or genes known to be expressed in particular cancer types and subtypes (i.e., different degrees of malignancy). This information permits a more informed and effective regimen of therapy to be administered, because drugs with clinical efficacy for certain tumor types or subtypes can be administered to patients whose cells are so identified.

For example, expression and activation of RAS proteins expressed from tumor-related genes can be detected and quantitated using methods of the present disclosure. Further, expression and activation of proteins that are cellular components of a tumor-related signaling pathway can be detected and quantitated using methods of the present disclosure. Further, proteins associated with cancer can be quantified by image analysis using a suitable primary antibody against biomarkers, such as, but not limited to, Her-1, Her-2, p-Her-1, Her-3, or p-ERK, and a secondary antibody (such as rabbit anti-mouse IgG when using mouse primary antibodies) and/or a tertiary avidin (or Strepavidin) biotin complex (“ABC”).

In practicing the method of the present disclosure, staining procedures can be carried out by a technician in the laboratory. Alternatively, the staining procedures can be carried out using automated systems. In either case, staining procedures for use according to the methods of this disclosure are performed according to standard techniques and protocols well-established in the art.

The amount of RAS can then be quantitated by the average optical density of the stained antigens. Also, the proportion or percentage of total tissue area stained may be readily calculated, as the area stained above an antibody threshold level in the second image. Following visualization of nuclei containing RAS, the percentage or amount of such cells in tissue derived from patients after treatment may be compared to the percentage or amount of such cells in untreated tissue or said tissue prior to treatment.

### Detection/Quantitation of RAS Amplification

The present invention encompasses methods of detection and/or quantitation of gene amplification (e.g., more than two copies of a gene including, for example, 3, 4, 5, 6, 7, 8, 9, 10, 11, 12, 13, 14, 15, 16, 17, 18, 19, 20 or more copies of the gene) known to those of skill in the art, see, for example, Boxer, J. Clin. Pathol. 53: 19-21 (2000). Such techniques include in situ hybridization (Stoler, Clin. Lab. Med. 12:215-36 (1990), using radioisotope or fluorophore-labeled probes; polymeRASe chain reaction (PCR); quantitative Southern blotting, dot blotting and other techniques for quantitating individual genes. Preferably, probes or primers selected for gene amplification evaluation are highly specific, to avoid detecting closely related homologous genes. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

In one embodiment, the biological sample contains nucleic acids from the test subject. The nucleic acids may be mRNA or genomic DNA molecules from the test subject.

1. Amplification Based Assays

In one embodiment of the present invention, amplification-based assays can be used to measure copy number of the RAS gene. In such amplification-based assays, the corresponding RAS nucleic acid sequence acts as a template in an amplification reaction (for example, PolymeRASe Chain Reaction or PCR). In a quantitative amplification, the amount of amplification product will be proportional to the amount of template in the original sample. Comparison to appropriate controls provides a measure of the copy-number of the RAS gene, corresponding to the specific probe used. The presence of a higher level of amplification product, as compared to a control, is indicative of amplified RAS.

a. Quantitative PCR

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided, for example, in Innis et al. (1990) PCR Protocols, A Guide to Methods and Applications, Academic Press, Inc. N.Y. The known nucleic acid sequence for the Met (Accession No.: NM 000245) is sufficient to enable one of skill to routinely select primers to amplify any portion of the RAS gene.

b. Real Time PCR

Real time PCR is another amplification technique that can be used to determine gene copy levels or levels of RAS mRNA expression. (See, e.g., Gibson et al., Genome Research 6:995-1001, 1996; Heid et al., Genome Research 6:986-994, 1996). Real-time PCR evaluates the level of PCR product accumulation during amplification. This technique permits quantitative evaluation of mRNA levels in multiple samples. For gene copy levels, total genomic DNA is isolated from a sample. For mRNA levels, mRNA is extracted from tumor and normal tissue and cDNA is prepared using standard techniques. Real-time PCR can be performed, for example, using a Perkin Elmer/Applied Biosystems (Foster City, Calif.) 7700 Prism instrument. Matching primers and fluorescent probes can be designed for genes of interest using, for example, the primer express program provided by Perkin Elmer/Applied Biosystems (Foster City, Calif.). Optimal concentrations of primers and probes can be initially determined by those of ordinary skill in the art, and control (for example, beta-actin) primers and probes may be obtained commercially from, for example, Perkin Elmer/Applied Biosystems (Foster City, Calif.). To quantitate the amount of the specific nucleic acid of interest in a sample, a standard curve is generated using a control. Standard curves may be generated using the Ct values determined in the real-time PCR, which are related to the initial concentration of the nucleic acid of interest used in the assay. Standard dilutions ranging from 10-10⁶ copies of the gene of interest are generally sufficient. In addition, a standard curve is generated for the control sequence. This permits standardization of initial content of the nucleic acid of interest in a tissue sample to the amount of control for comparison purposes.

Methods of real-time quantitative PCR using TaqMan probes are well known in the art. Detailed protocols for real-time quantitative PCR are provided, for example, for RNA in: Gibson et al., 1996, A novel method for real time quantitative RT-PCR. Genome Res., 10:995-1001; and for DNA in: Heid et al., 1996, Real time quantitative PCR. Genome Res., 10:986-994.

A TaqMan-based assay also can be used to quantify MET polynucleotides. TaqMan based assays use a fluorogenic oligonucleotide probe that contains a 5′ fluorescent dye and a 3′ quenching agent. The probe hybridizes to a PCR product, but cannot itself be extended due to a blocking agent at the 3′ end. When the PCR product is amplified in subsequent cycles, the 5′ nuclease activity of the polymeRASe, for example, AmpliTaq, results in the cleavage of the TaqMan probe. This cleavage separates the 5′ fluorescent dye and the 3′ quenching agent, thereby resulting in an increase in fluorescence as a function of amplification.

c. Other Amplification Methods

Other suitable amplification methods include, but are not limited to ligase chain reaction (LCR) (see Wu and Wallace (1989) Genomics 4:560, Landegren et al. (1988) Science 241:1077, and Barringer et al. (1990) Gene 89:117), transcription amplification (Kwoh et al. (1989) Proc. Natl. Acad. Sci. USA 86:1173), self-sustained sequence replication (Guatelli et al. (1990) Proc. Nat. Acad. Sci. USA 87:1874), dot PCR, and linker adapter PCR, etc.

2. Hybridization Based Assays

Hybridization assays can be used to detect RAS copy number. Hybridization-based assays include, but are not limited to, traditional “direct probe” methods such as Southern blots or in situ hybridization (e.g., FISH), and “comparative probe” methods such as comparative genomic hybridization (CGH). The methods can be used in a wide variety of formats including, but not limited to substrate—(e.g. membrane or glass) bound methods or array-based approaches as described below.

a. Southern Blot

One method for evaluating the copy number of RAS encoding nucleic acid in a sample involves a Southern transfer. Methods for doing Southern Blots are known to those of skill in the art (see Current Protocols in Molecular Biology, Chapter 19, Ausubel, et al., Eds., Greene Publishing and Wiley-Interscience, New York, 1995, or Sambrook et al., Molecular Cloning: A Laboratory Manual, 2d Ed. vol. 1-3, Cold Spring Harbor Press, NY, 1989). In such an assay, the genomic DNA (typically fragmented and separated on an electrophoretic gel) is hybridized to a probe specific for the target region. Comparison of the intensity of the hybridization signal from the probe for the target region with control probe signal from analysis of normal genomic DNA (e.g., a non-amplified portion of the same or related cell, tissue, organ, etc.) provides an estimate of the relative copy number of the target nucleic acid. An intensity level that is higher than the control is indicative of amplified RAS.

b. Fluorescence in Situ Hybridization (FISH)

In another embodiment, FISH is used to determine the copy number of the RAS gene in a sample. Fluorescence in situ hybridization (FISH) is known to those of skill in the art (see Angerer, 1987 Meth. Enzymol., 152: 649). Generally, in situ hybridization comprises the following major steps: (1) fixation of tissue or biological structure to be analyzed; (2) pre-hybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization, and (5) detection of the hybridized nucleic acid fragments.

In a typical in situ hybridization assay, cells or tissue sections are fixed to a solid support, typically a glass slide. If a nucleic acid is to be probed, the cells are typically denatured with heat or alkali. The cells are then contacted with a hybridization solution at a moderate temperature to permit annealing of labeled probes specific to the nucleic acid sequence encoding the protein. The targets (e.g., cells) are then typically washed at a predetermined stringency or at an increasing stringency until an appropriate signal to noise ratio is obtained.

The probes used in such applications are typically labeled, for example, with radioisotopes or fluorescent reporters. Preferred probes are sufficiently long, for example, from about 50, 100, or 200 nucleotides to about 1000 or more nucleotides, to enable specific hybridization with the target nucleic acid(s) under stringent conditions.

In some applications it is necessary to block the hybridization capacity of repetitive sequences. Thus, in some embodiments, tRNA, human genomic DNA, or Cot-1 DNA is used to block non-specific hybridization. Thus, in one embodiment of the present invention, the presence or absence of RAS amplification is determined by FISH.

c. Comparative Genomic Hybridization (CGH)

In comparative genomic hybridization methods, a “test” collection of nucleic acids (e.g. from a possible tumor) is labeled with a first label, while a second collection (e.g. from a normal cell or tissue) is labeled with a second label. The ratio of hybridization of the nucleic acids is determined by the ratio of the first and second labels binding to each fiber in an array. Differences in the ratio of the signals from the two labels, for example, due to gene amplification in the test collection, is detected and the ratio provides a measure of the gene copy number, corresponding to the specific probe used. A cytogenetic representation of DNA copy-number variation can be generated by CGH, which provides fluorescence ratios along the length of chromosomes from differentially labeled test and reference genomic DNAs. In another embodiment of the present invention, comparative genomic hybridization may be used to detect the presence or absence of RAS amplification.

d. Microarray Based Comparative Genomic Hybridization

In an alternative embodiment of the present invention, DNA copy numbers are analyzed via microarray-based platforms. Microarray technology offers high resolution. For example, the traditional CGH generally has a 20 Mb limited mapping resolution; whereas in microarray-based CGH, the fluorescence ratios of the differentially labeled test and reference genomic DNAs provide a locus-by-locus measure of DNA copy-number variation, thereby achieving increased mapping resolution. Details of various microarray methods can be found in the literature. See, for example, U.S. Pat. No. 6,232,068; Pollack et al., Nat. Genet., 23(1):41-6, (1999), Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211 and others.

The DNA used to prepare the arrays of the invention is not critical. For example, the arrays can include genomic DNA, e.g. overlapping clones that provide a high resolution scan of a portion of the genome containing the desired gene, or of the gene itself. Genomic nucleic acids can be obtained from, e.g., HACs, MACs, YACs, BACs, PACs, P1s, cosmids, plasmids, inter-Alu PCR products of genomic clones, restriction digests of genomic clones, cDNA clones, amplification (e.g., PCR) products, and the like. Arrays can also be produced using oligonucleotide synthesis technology. Thus, for example, U.S. Pat. No. 5,143,854 and PCT Patent Publication Nos. WO 90/15070 and WO 92/10092 teach the use of light-directed combinatorial synthesis of high density oligonucleotide arrays.

Hybridization protocols suitable for use with the methods of the invention are described, e.g., in Albertson (1984) EMBO J. 3: 1227-1234; Pinkel (1988) Proc. Natl. Acad. Sci. USA 85: 9138-9142; EPO Pub. No. 430,402; Methods in Molecular Biology, Vol. 33: In situ Hybridization Protocols, Choo, ed., Humana Press, Totowa, N.J. (1994), Pinkel et al. (1998) Nature Genetics 20: 207-211, or of Kallioniemi (1992) Proc. Natl. Acad Sci USA 89:5321-5325 (1992), etc.

The sensitivity of the hybridization assays may be enhanced through use of a nucleic acid amplification system that multiplies the target nucleic acid being detected. Examples of such systems include the polymeRASe chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

In another embodiment of the present invention, kits useful for the detection of Met amplification are disclosed. Such kits may include any or all of the following: assay reagents, buffers, specific nucleic acids or antibodies (e.g. full-size monoclonal or polyclonal antibodies, single chain antibodies (e.g., scFv), or other gene product binding molecules), and other hybridization probes and/or primers, and/or substrates for polypeptide gene products.

In addition, the kits may include instructional materials containing directions (i.e., protocols) for the practice of the methods of this invention. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### Methods for Predicting Sensitivity to a Drug

The present disclosure provides methods for predicting sensitivity of a test cell to a DHFR inhibitor (e.g., an antifolate such as Methotrexate or Pemetrexed (ALTIMA™)) by obtaining a test cell; contacting the test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to predict sensitivity of the test cell to the DHFR inhibitor, wherein the test cell has mutated RAS and no amplification of the RAS gene. The test cell may be predicted to be sensitive to the DHFR inhibitor where expression of the one or more miRNAs (e.g., miR-181c and/or miR143) in the test cell is elevated as compared to expression of the one or more miRNAs in the control cell or is above the threshold. The threshold may be set at a level of miRNA expression above which a control cell is known to be sensitive to treatment with the DHFR inhibitor and below which a control cell is known to not be sensitive to treatment with the DHFR inhibitor. In some embodiments, the threshold may be set at a level of miRNA expression above which 50%, 60%, 70%, 80%, 90%, or 95% of control cells respond to treatment with a DHFR or at a level of miRNA expression below which 50%, 60%, 70%, 80%, 90%, or 95% of control cells do not respond to treatment with a DHFR inhibitor.

In some embodiments, a test cell is sensitive to a DHFR inhibitor where the mean GI50 of the DHFR inhibitor is 0.10 μM or less (e.g., 0.10 μM, 0.50 μM, or 0.10 μM). In some embodiments, the threshold is set at a mean GI50 (e.g., 0.10 μM, 0.50 μM, or 0.10 μM) of the DHFR inhibitor.

The present disclosure provides methods for predicting sensitivity of a test cell (e.g., a cell obtained from a cancer patient) to a drug (e.g., an antifolate such as a dihydrofolate reductase (DHFR); or an EGFR inhibitor) by obtaining a test cell; assaying the test cell for one or more RAS mutations (e.g., one or more mutations in KRAS (SEQ ID NO: 1), NRAS (SEQ ID NO: 2) or HRAS (SEQ ID NO: 3); assaying the test cell for amplification of a RAS gene; determining if one or more RAS mutations are present or absent in the test cell and determining if an amplification of the RAS gene is present or absent in the test cell; and employing the determination of the presence or absence of a RAS mutation in the test cell and the presence or absence of an amplification of RAS in the test cell to predict sensitivity of the test cell to the drug. In some embodiments, the test cell is predicted to be sensitive to the drug where one or more RAS mutations are determined to be present in the test cell and an amplification of RAS is determined to be present in the test cell. In some embodiments, the test cell is predicted to be sensitive to the drug where one or more RAS mutations are determined to be present in the test cell and amplification of RAS is determined to be absent in the test cell. In some embodiments, the test cell is predicted to be sensitive to the drug where RAS mutations are determined to be absent (e.g., RAS wild type) in the test cell and an amplification of RAS is determined to be present in the test cell. In some embodiments, the test cell is predicted to be sensitive to the drug where RAS mutations are determined to be absent (e.g., RAS wild type) in the test cell and amplification of RAS is determined to be absent in the test cell. In some embodiments, the test cell is predicted to be insensitive to the drug where one or more RAS mutations are determined to be present in the test cell and an amplification of RAS is determined to be present in the test cell. In some embodiments, the test cell is predicted to be insensitive to the drug where one or more RAS mutations are determined to be present in the test cell and amplification of RAS is determined to be absent in the test cell. In some embodiments, the test cell is predicted to be insensitive to the drug where RAS mutations are determined to be absent (e.g., RAS wild type) in the test cell and an amplification of RAS is determined to be present in the test cell. In some embodiments, the test cell is predicted to be insensitive to the drug where RAS mutations are determined to be absent (e.g., RAS wild type) in the test cell and amplification of RAS is determined to be absent in the test cell.

In some embodiments, the test cell is predicted to be sensitive to the drug where the number of RAS mutations in the test cell is elevated as compared to the number of RAS mutations in a control cell or is above a threshold. In some embodiments, the test cell is predicted to be sensitive to the drug where the number of RAS mutations in the test cell is reduced as compared to the number of RAS mutations in a control cell or is above a threshold. In some embodiments, the test cell is predicted to be sensitive to the drug where the number of amplifications of RAS in the test cell is elevated as compared to the number of amplifications of RAS in a control cell or is above a threshold. In some embodiments, the test cell is predicted to be sensitive to the drug where the number of amplifications of RAS in the test cell is reduced as compared to the number of amplifications of RAS in a control cell or is above a threshold. In some embodiments, the test cell is predicted to be sensitive to the drug where the number of RAS mutations is elevated and number of amplifications of RAS in the test cell is elevated as compared to the number of RAS mutations and number of amplifications of RAS in a control cell or is above a threshold. In some embodiments, the test cell is predicted to be sensitive to the drug where the number of RAS mutations is reduced and number of amplifications of RAS in the test cell is elevated as compared to the number of RAS mutations and number of amplifications of RAS in a control cell or is above a threshold. In some embodiments, the test cell is predicted to be sensitive to the drug where the number of RAS mutations is elevated and number of amplifications of RAS in the test cell is reduced as compared to the number of RAS mutations and number of amplifications of RAS in a control cell or is above a threshold. In some embodiments, the test cell is predicted to be insensitive to the drug where the number of RAS mutations in the test cell is elevated as compared to the number of RAS mutations in a control cell or is above a threshold. In some embodiments, the test cell is predicted to be insensitive to the drug where the number of RAS mutations in the test cell is reduced as compared to the number of RAS mutations in a control cell or is above a threshold. In some embodiments, the test cell is predicted to be insensitive to the drug where the number of amplifications of RAS in the test cell is elevated as compared to the number of amplifications of RAS in a control cell or is above a threshold. In some embodiments, the test cell is predicted to be insensitive to the drug where the number of amplifications of RAS in the test cell is reduced as compared to the number of amplifications of RAS in a control cell or is above a threshold. In some embodiments, the test cell is predicted to be insensitive to the drug where the number of RAS mutations is elevated and number of amplifications of RAS in the test cell is elevated as compared to the number of RAS mutations and number of amplifications of RAS in a control cell or is above a threshold. In some embodiments, the test cell is predicted to be insensitive to the drug where the number of RAS mutations is reduced and number of amplifications of RAS in the test cell is elevated as compared to the number of RAS mutations and number of amplifications of RAS in a control cell or is above a threshold. In some embodiments, the test cell is predicted to be insensitive to the drug where the number of RAS mutations is elevated and number of amplifications of RAS in the test cell is reduced as compared to the number of RAS mutations and number of amplifications of RAS in a control cell or is above a threshold.

In some embodiments, the threshold may be set at a number of RAS mutations and/or level of expression of mutated RAS and/or number of RAS amplifications above which a control cell is known to be sensitive to treatment with the drug and below which the control cell is known to not be sensitive to treatment with the drug.

In some embodiments, the threshold is set at a number of RAS mutations and/or level of expression of mutated RAS and/or number of RAS amplifications above which 50%, 60%, 70%, 80%, 90%, or 95% of control cells respond to treatment with the drug.

In some embodiments, the threshold is set at a number of RAS mutations and/or level of expression of mutated RAS and/or number of RAS amplifications below which 50%, 60%, 70%, 80%, 90%, or 95% of control cells do not respond to treatment with the drug.

Methods for Screening and/or Identifying a Drug

Methods for screening and identifying a drug including, for example, a DHFR inhibitor, are provided by the disclosure. Such methods may be used to screen and/or identify a drug including, for example, a DHFR inhibitor, that has a pharmacological activity (e.g., exerts a cytotoxic effect) or identify a drug including, for example, a DHFR inhibitor, that inhibits Ras activity.

The present disclosure provides methods for screening one or more dihydrofolate reductase (DHFR) inhibitors for a pharmacological activity, comprising: contacting a test cell with the one or more DHFR inhibitors; assaying the contacted test cell for expression of one or more miRNAs; determining if expression of the one or more miRNAs in the test cell is elevated or reduced compared to a control cell or above or below a threshold; and employing the determination of the expression of the one or more miRNAs in the test cell to determine a pharmacological activity of the DHFR inhibitor, wherein the test cell has mutated RAS and no amplification of the RAS gene. Optionally, the methods may further comprise obtaining the test cell. The threshold may be set at a level of miRNA expression in a control cell above which a cell (e.g., test cell) is known to have a certain pharmacological activity and below which a cell is known to not have a certain pharmacological activity. In some embodiments, the threshold is set at a level of miRNA expression in a control cell above which 50%, 60%, 70%, 80%, 90%, or 95% of cells have a certain pharmacological activity. In some embodiments, the threshold is set at a level of miRNA expression in a control cell below which 50%, 60%, 70%, 80%, 90%, or 95% of cells do not have a certain pharmacological activity. For example, a threshold may be set at the minimal amount of expression of one or more miRNAs in a control cell where the control cell has a certain pharmacological activity. Such a threshold may be an average or median obtained from two or more control cells. In some embodiments, the test cell may be predicted or determined to have a certain pharmacological activity where the level of expression one or more miRNAs in the cells (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the level of expression of one or more miRNAs detected in a control cell. In some embodiments, the test cell may be predicted or determined to have a certain pharmacological activity where the level of expression of one or more miRNAs in the test cell (e.g., tumor cells in the biological sample) is 2 times, 3 times, 4 times, 5 times, 6 times, 7 times, 8 times, 9 times, 10 times or more than the level of expression of one or more miRNAs in a control cell. In some embodiments, the test cell and control cell are from the same specimen. In some embodiments, the test cell and control cell are from the different specimens.

The present disclosure also provides methods of identifying a dihydrofolate reductase (DHFR) inhibitor that inhibits Ras activity, comprising, contacting the test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to determine if the DHFR inhibitor inhibits Ras activity, wherein the test cell has mutated RAS and no amplification of the RAS gene. Optionally, the methods may further comprise obtaining the test cell. The threshold may be set at a level of miRNA expression in a control cell above which a cell (e.g., test cell) is known to have Ras activity and below which a cell is known to not have Ras activity. In some embodiments, the threshold is set at a level of miRNA expression in a control cell above which 50%, 60%, 70%, 80%, 90%, or 95% of cells have Ras activity. In some embodiments, the threshold is set at a level of miRNA expression in a control cell below which 50%, 60%, 70%, 80%, 90%, or 95% of cells do not have Ras activity. For example, a threshold may be set at the minimal amount of expression of one or more miRNAs in a control cell where the control cell has Ras activity. Such a threshold may be an average or median obtained from two or more control cells. In some embodiments, the test cell may be predicted or determined to have Ras activity where the level of expression one or more miRNAs in the cells (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the level of expression of one or more miRNAs detected in a control cell. In some embodiments, the test cell may be predicted or determined to have Ras activity where the level of expression of one or more miRNAs in the test cell (e.g., tumor cells in the biological sample) is 2 times, 3 times, 4 times, 5 times, 6 times, 7 times, 8 times, 9 times, 10 times or more than the level of expression of one or more miRNAs in a control cell. In some embodiments, the test cell and control cell are from the same specimen. In some embodiments, the test cell and control cell are from the different specimens.

The present disclosure also provides methods of identifying a dihydrofolate reductase (DHFR) inhibitor that is toxic to a test cell, comprising, contacting a test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to determine if the DHFR inhibitor is toxic to the cell, wherein the test cell has mutated RAS and no amplification of the RAS gene. Optionally, the methods may further comprise obtaining the test cell. The threshold may be set at a level of miRNA expression in a control cell above which a cell (e.g., test cell) is known to be toxic and below which a cell is known to not be toxic. In some embodiments, the threshold is set at a level of miRNA expression in a control cell above which 50%, 60%, 70%, 80%, 90%, or 95% of cells are toxic. In some embodiments, the threshold is set at a level of miRNA expression in a control cell below which 50%, 60%, 70%, 80%, 90%, or 95% of cells are not toxic. For example, a threshold may be set at the minimal amount of expression of one or more miRNAs in a control cell where the control cell is toxic. Such a threshold may be an average or median obtained from two or more control cells. In some embodiments, the test cell may be predicted or determined to be toxic where the level of expression one or more miRNAs in the cells (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the level of expression of one or more miRNAs detected in a control cell. In some embodiments, the test cell may be predicted or determined to be toxic where the level of expression of one or more miRNAs in the test cell (e.g., tumor cells in the biological sample) is 2 times, 3 times, 4 times, 5 times, 6 times, 7 times, 8 times, 9 times, 10 times or more than the level of expression of one or more miRNAs in a control cell. In some embodiments, the test cell and control cell are from the same specimen. In some embodiments, the test cell and control cell are from the different specimens.

In various embodiments, two or more drugs may be screened against a single cell type on a substrate (e.g., screened on an array). In alternative embodiments, two or more cells may be screened against a single drug on a substrate (e.g., screened on an array). In other embodiments, two or more drugs may be screened against two or more cell types on a substrate (e.g., screened on an array).

Methods for Predicting and/or Determining Responsiveness of a Subject to a DHFR Inhibitor

Methods for predicting and/or determining the responsiveness of a subject to a DHFR inhibitor such as an antifolate are provided by the present disclosure. Such methods may be used to select patients predicted or determined to be responsive to a DHFR inhibitor for treatment with a DHFR inhibitor, including, for a clinical trial testing the safety and/or efficacy of a DHFR inhibitor.

The present disclosure provides methods for predicting and/or determining responsiveness of a subject with a disease or disorder to treatment with a DHFR inhibitor (e.g., an antifolate such as Methotrexate or Pemetrexed (ALTIMA™)) by administering a DHFR inhibitor to the subject; obtaining a biological sample from the subject; assaying the biological sample for expression of one or more miRNAs; determining if expression of the one or more miRNAs in the biological sample is elevated or reduced compared to a control sample or above or below a threshold; and employing the determination of the expression of the one or more miRNAs in the biological sample to predict responsiveness of the subject to the DHFR inhibitor, wherein the biological sample comprises cells with mutated RAS and no amplification of the RAS gene. The subject may be predicted or determined to be responsive to the DHFR inhibitor where expression of the one or more miRNAs (e.g., miR-181c and/or miR143) in the biological sample is elevated as compared to the control sample or is greater than the threshold. The threshold may be set at a level of miRNA expression in a control sample above which a subject is known to respond to treatment with the DHFR inhibitor and below which a subject is known to not respond to treatment with the DHFR inhibitor. In some embodiments, the threshold is set at a level of miRNA expression in the control sample above which 50%, 60%, 70%, 80%, 90%, or 95% of subject(s) respond to treatment with the DHFR inhibitor. In some embodiments, the threshold is set at a level of miRNA expression in the control sample below which 50%, 60%, 70%, 80%, 90%, or 95% of subject(s) do not respond to treatment with the DHFR inhibitor. For example, a threshold may be set at the minimal amount of expression of one or more miRNAs in a biological sample obtained from a subject where the subject is responsive to treatment with a drug. Such a threshold may be an average or median obtained from two or more subjects. In some embodiments, the subject may be predicted or determined to be responsive to a drug where the level of expression one or more miRNAs in a biological sample (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the level of expression of one or more miRNAs detected in a control sample. In some embodiments, the subject may be predicted or determined to be responsive to a drug where the level of expression of one or more miRNAs in a biological sample (e.g., tumor cells in the biological sample) is 2 times, 3 times, 4 times, 5 times, 6 times, 7 times, 8 times, 9 times, 10 times or more than the level of expression of one or more miRNAs in a biological sample detected in a control sample. In some embodiments, the biological sample and control sample are from the same specimen. In some embodiments, the biological sample and control sample are from the different specimens.

The present disclosure also provides methods for treating a subject with a disease or disorder, the method comprising: administering a DHFR inhibitor (e.g., an antifolate such as Methotrexate or Pemetrexed (ALTIMA™)) to the subject; obtaining a biological sample from a subject; assaying the biological sample for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and administering to the subject a therapeutically effective amount of DHFR inhibitor where the subject is predicted to be responsive to the DHFR inhibitor, wherein the biological sample comprises cells with mutated RAS and no amplification of the RAS gene. The subject may be predicted or determined to be responsive to the DHFR inhibitor where expression of the one or more miRNAs (e.g., miR-181c and/or miR143) in the biological sample is elevated as compared to the control sample or is greater than the threshold. The threshold may be set at a level of miRNA expression in a control sample above which a subject is known to respond to treatment with the DHFR inhibitor and below which a subject is known to not respond to treatment with the DHFR inhibitor. In some embodiments, the threshold is set at a level of miRNA expression in the control sample above which 50%, 60%, 70%, 80%, 90%, or 95% of subject(s) respond to treatment with the DHFR inhibitor. In some embodiments, the threshold is set at a level of miRNA expression in the control sample below which 50%, 60%, 70%, 80%, 90%, or 95% of subject(s) do not respond to treatment with the DHFR inhibitor. For example, a threshold may be set at the minimal amount of expression of one or more miRNAs in a biological sample obtained from a subject where the subject is responsive to treatment with a drug. Such a threshold may be an average or median obtained from two or more subjects. In some embodiments, the subject may be predicted or determined to be responsive to a drug where the level of expression one or more miRNAs in a biological sample (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the level of expression of one or more miRNAs detected in a control sample. In some embodiments, the subject may be predicted or determined to be responsive to a drug where the level of expression of one or more miRNAs in a biological sample (e.g., tumor cells in the biological sample) is 2 times, 3 times, 4 times, 5 times, 6 times, 7 times, 8 times, 9 times, 10 times or more than the level of expression of one or more miRNAs in a biological sample detected in a control sample. In some embodiments, the biological sample and control sample are from the same specimen. In some embodiments, the biological sample and control sample are from the different specimens.

The present disclosure also provides methods for selecting subjects for a clinical trial for testing the efficacy or safety of a dihydrofolate reductase (DHFR) inhibitor (e.g., an antifolate such as Methotrexate or Pemetrexed (ALTIMA™)), the method comprising: administering a DHFR inhibitor to the subject; obtaining a biological sample from the subjects; assaying the biological samples obtained from the subjects for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and selecting subjects for inclusion in a clinical trial that are responsive to the DHFR inhibitor, wherein the biological sample comprises one or more cells with mutated RAS and no amplification of the RAS gene. The subject may be predicted or determined to be responsive to the DHFR inhibitor where expression of the one or more miRNAs (e.g., miR-181c and/or miR143) in the biological sample is elevated as compared to the control sample or is greater than the threshold. The threshold may be set at a level of miRNA expression in a control sample above which a subject is known to respond to treatment with the DHFR inhibitor and below which a subject is known to not respond to treatment with the DHFR inhibitor. In some embodiments, the threshold is set at a level of miRNA expression in the control sample above which 50%, 60%, 70%, 80%, 90%, or 95% of subject(s) respond to treatment with the DHFR inhibitor. In some embodiments, the threshold is set at a level of miRNA expression in the control sample below which 50%, 60%, 70%, 80%, 90%, or 95% of subject(s) do not respond to treatment with the DHFR inhibitor. For example, a threshold may be set at the minimal amount of expression of one or more miRNAs in a biological sample obtained from a subject where the subject is responsive to treatment with a drug. Such a threshold may be an average or median obtained from two or more subjects. In some embodiments, the subject may be predicted or determined to be responsive to a drug where the level of expression one or more miRNAs in a biological sample (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the level of expression of one or more miRNAs detected in a control sample. In some embodiments, the subject may be predicted or determined to be responsive to a drug where the level of expression of one or more miRNAs in a biological sample (e.g., tumor cells in the biological sample) is 2 times, 3 times, 4 times, 5 times, 6 times, 7 times, 8 times, 9 times, 10 times or more than the level of expression of one or more miRNAs in a biological sample detected in a control sample. In some embodiments, the biological sample and control sample are from the same specimen. In some embodiments, the biological sample and control sample are from the different specimens.

The present disclosure also provides methods for predicting and/or determining responsiveness of a subject with a disease or disorder to treatment with a drug (e.g., an antifolate such as a dihydrofolate reductase (DHFR); or an EGFR inhibitor) by obtaining a biological sample (e.g., a biological sample obtained from a cancer patient such as a formalin fixed paraffin embedded tissue) from the subject; assaying target cells obtained from the biological sample for one or more RAS mutations (e.g., one or more mutations in KRAS (SEQ ID NO: 1), NRAS (SEQ ID NO: 2) or HRAS (SEQ ID NO: 3); assaying target cells obtained from the biological sample for a RAS amplification; determining if one or more RAS mutations are present or absent in the target cells and determining if an amplification of the RAS gene is present or absent in the target cells; and employing the determination of the presence or absence of a RAS mutation and the presence or absence of an amplification of RAS in the target cells obtained from the biological sample to predict responsiveness of the subject to the drug. In some embodiments, the subject is predicted to be responsive to the drug where one or more RAS mutations are present in the target cells and an amplification of RAS is present in the target cells. In some embodiments, the subject is predicted or determined to be responsive to the drug where one or more RAS mutations are absent (e.g., RAS wild type) in the target cells and an amplification of RAS is present in the target cells. In some embodiments, the subject is predicted to be responsive to the drug where one or more RAS mutations are present in the target cells and an amplification of RAS is absent in the target cells. In some embodiments, the subject is predicted or determined to be responsive to the drug where one or more RAS mutations are absent (e.g., RAS wild type) in the target cells and an amplification of RAS is absent in the target cells. In some embodiments, the subject is predicted or determined to be non-responsive to the drug where one or more RAS mutations are present in the target cells and an amplification of RAS is present in the target cells. In some embodiments, the subject is predicted or determined to be non-responsive to the drug where one or more RAS mutations are absent (e.g., RAS wild type) in the target cells and an amplification of RAS is present in the target cells. In some embodiments, the subject is predicted or determined to be non-responsive to the drug where one or more RAS mutations are present in the target cells and an amplification of RAS is absent in the target cells. In some embodiments, the subject is predicted or determined to be non-responsive to the drug where one or more RAS mutations are absent (e.g., RAS wild type) in the target cells and an amplification of RAS is absent in the target cells.

The present disclosure also provides methods for treating a subject with a disease or disorder by obtaining a biological sample (e.g., a biological sample obtained from a cancer patient) from a subject; assaying target cells obtained from the biological sample for one or more RAS mutations (e.g., one or more mutations in KRAS (SEQ ID NO: 1), NRAS (SEQ ID NO: 2) or HRAS (SEQ ID NO: 3); assaying target cells obtained from the biological sample for a RAS amplification (e.g., an amplification of one or more of KRAS (SEQ ID NO: 4), NRAS (SEQ ID NO: 5) or HRAS (SEQ ID NO: 6)); determining if one or more RAS mutations are present or absent in the target cells and determining if an amplification of the RAS gene is present or absent in the target cells; employing the determination of the presence or absence of a RAS mutation and the presence or absence of an amplification of RAS in the target cells obtained from the biological sample to predict responsiveness of the subject to a drug (e.g., an antifolate such as a dihydrofolate reductase (DHFR); or an EGFR inhibitor); and administering to the subject a therapeutically effective amount of the drug where the subject is predicted to be responsive to the drug. In some embodiments, the subject is predicted to be responsive to the drug where one or more RAS mutations are present in the target cells and an amplification of RAS is present in the target cells. In some embodiments, the subject is predicted to be responsive to the drug where one or more RAS mutations are absent (e.g., RAS wild type) in the target cells and an amplification of RAS is present in the target cells. In some embodiments, the subject is predicted to be responsive to the drug where one or more RAS mutations are present in the target cells and an amplification of RAS is absent in the target cells. In some embodiments, the subject is predicted to be responsive to the drug where one or more RAS mutations are absent (e.g., RAS wild type) in the target cells and an amplification of RAS is absent in the target cells.

The present disclosure also provides methods for selecting subjects for inclusion in a clinical trial for testing the efficacy or safety of a drug (e.g., an antifolate such as a dihydrofolate reductase (DHFR); or an EGFR inhibitor) by obtaining a biological sample (e.g., a biological sample obtained from a cancer patient) comprising target cells from the subject; assaying target cells in the biological sample for one or more RAS mutations (e.g., one or more mutations in KRAS (SEQ ID NO: 1), NRAS (SEQ ID NO: 2) or HRAS (SEQ ID NO: 3); assaying target cells in the biological sample for an amplification of RAS (e.g., an amplification of one or more of KRAS (SEQ ID NO: 4), NRAS (SEQ ID NO: 5) or HRAS (SEQ ID NO: 6)); determining if one or more RAS mutations are present or absent in the target cells and determining if an amplification of the RAS gene is present or absent in the target cells; employing the determination of the presence or absence of a RAS mutation in the target cells and the presence or absence of an amplification of RAS in the target cells to predict sensitivity of the target cells to the drug; and selecting subjects for inclusion in the clinical that are predicted to be responsive to the drug. In some embodiments, subjects are selected for the clinical trial that have one or more RAS mutations present in target cells from their biological sample and that have an amplification of RAS present in target cells from their biological sample. In some embodiments, subjects are selected for the clinical trial that have one or more RAS mutations absent (e.g., RAS wild type) in target cells from their biological sample and that have an amplification of RAS present in target cells from their biological sample. In some embodiments, subjects are selected for the clinical trial that have one or more RAS mutations present in target cells from their biological sample and that have an amplification of RAS absent in target cells from their biological sample. In some embodiments, subjects are selected for the clinical trial that have one or more RAS mutations absent (e.g., RAS wild type) in target cells from their biological sample and that have an amplification of RAS absent in target cells from their biological sample.

The subject may be predicted to be responsive to the drug where the number of RAS mutations and/or number of RAS amplifications in the biological sample is elevated as compared to the control sample or is greater than a threshold. Alternatively, the subject may be predicted to not be responsive to the drug where the number of RAS mutations and/or number of RAS amplifications in the biological sample is reduced as compared to the control sample or is less than the threshold. The threshold may be set at a number of RAS mutations and/or number of RAS amplifications above which the control sample is known to respond to treatment with the drug and below which a control sample is known to not respond to treatment with the drug. In some embodiments, the threshold may be set at the number of RAS mutations and/or number of RAS amplifications above which 50%, 60%, 70%, 80%, 90%, or 95% of control samples respond to treatment with a drug and/or at a number of RAS mutations and/or number of RAS amplifications below which 50%, 60%, 70%, 80%, 90%, or 95% of control samples do not respond to treatment with a drug. Alternatively, a subject may be predicted to be responsive to a drug where the expression (e.g., amount or level) of mutant RAS and/or the number of RAS amplifications detected in the biological sample is above or below a set threshold. For example, a threshold may be set at the maximum amount of expression of mutated RAS and/or number of RAS amplifications in a biological sample obtained from a subject where the subject is responsive to treatment with a drug. Such a threshold may be an average or median obtained from two or more subjects.

In some embodiments, the subject may be predicted to be responsive to a drug where the number of RAS mutations and/or level of mutant RAS expression and/or number of RAS amplifications in a biological sample (e.g., tumor cells in the biological sample) is 5%, 10%, 15%, 20%, 25%, 30%, 35%, 40%, 45%, 50%, 55%, 60%, 65%, 70%, 75%, 80%, 85%, 90%, 95%, 100%, 200% or more than the number of RAS mutations and/or level of mutant RAS expression and/or the number of RAS amplifications detected in a control sample. In some embodiments, the subject may be predicted to be responsive to a drug where the number of RAS mutations and/or level of mutant RAS expression and/or number of RAS amplifications in a biological sample (e.g., tumor cells in the biological sample) is 2 times, 3 times, 4 times, 5 times, 6 times, 7 times, 8 times, 9 times, 10 times or more than the number of RAS mutations and/or level of mutant RAS expression and/or number of RAS amplifications in a biological sample detected in a control sample. In some embodiments, the biological sample and control sample are from the same specimen. In some embodiments, the biological sample and control sample are from the different specimens.

In some embodiments, the threshold may be set at a number of RAS mutations and/or level of expression of mutated RAS and/or number of RAS amplifications above which a control cell is known to be sensitive to treatment with the drug and below which the control cell is known to not be sensitive to treatment with the drug.

In some embodiments, the threshold is set at a number of RAS mutations and/or level of expression of mutated RAS and/or number of RAS amplifications above which 50%, 60%, 70%, 80%, 90%, or 95% of control cells respond to treatment with the drug.

In some embodiments, the threshold is set at a number of RAS mutations and/or level of expression of mutated RAS and/or number of RAS amplifications below which 50%, 60%, 70%, 80%, 90%, or 95% of control cells do not respond to treatment with the drug.

The present disclosure also provides methods for modulating the responsiveness of a subject to an EGFR targeted therapy including, for example, a DHFR inhibitor such as Methotrexate by obtaining a biological sample comprising target cells from the subject, determining if the cells have one or more RAS mutations; determining if the cells have a RAS amplification, and where it is determined that the subject has a RAS mutation and does not have a RAS amplification; administering to the subject one or more agents that increase expression of RAS (e.g., KRAS).

### Pharmaceutical Formulations

Pharmaceutical formulations comprising one or more drugs including, for example, chemotherapeutic agents are provided. Such agents may include an antifolate including, for example, a dihydrofolate reductase (DHFR) inhibitor such as Methotrexate or Pemetrexed. Such agents may additionally or alternatively include a tyrosine kinase inhibitor that targets HER1 (EGFR), HER2/neu, HER3, or any combination thereof such as cetuximab (Erbitux), panitumumab, zalutumumab, nimotuzumab or matuzumab.

The drug can be administered as an active ingredient in admixture with suitable pharmaceutical diluents, excipients, or carriers (collectively referred to herein as “carrier” materials) suitably selected with respect to the intended form of administration, that is, oral tablets, capsules, elixirs, syrups and the like, and consistent with conventional pharmaceutical practices.

For example, in one embodiment, the pharmaceutical composition comprises a drug solution with L-arginine. To prepare this composition, a 10 g quantity of L-arginine was added to a vessel containing approximately 70 mL of Water-For-Injections BP. The mixture was stirred with a magnetic stirrer until the arginine had dissolved. A 5 g quantity of PXD-101 was added, and the mixture stirred at 25° C. until the PXD-101 had dissolved. The solution was diluted to a final volume of 100 mL using Water-For-Injections BP. The resulting solution had a pH of 9.2-9.4 and an osmolality of approximately 430 mOSmol/kg. The solution was filtered through a suitable 0.2 sterilizing (e.g., PVDF) membrane. The filtered solution was placed in vials or ampoules, which were sealed by heat, or with a suitable stopper and cap. The solutions were stored at ambient temperature, or, more preferably, under refrigeration (e.g., 2-8° C.) in order to reduced degradation of the drug.

In one embodiment, the drug can be administered orally. Oral administration can be in the form of a tablet or capsule. The drug can be combined with an oral, non-toxic, pharmaceutically acceptable, inert carrier such as lactose, starch, sucrose, glucose, methyl cellulose, microcrystalline cellulose, sodium croscarmellose, magnesium stearate, dicalcium phosphate, calcium sulfate, mannitol, sorbitol and the like or a combination thereof. For oral administration in liquid form, the drug can be combined with any oral, non-toxic, pharmaceutically acceptable inert carrier such as ethanol, glycerol, water and the like. Moreover, when desired or necessary, suitable binders, lubricants, disintegrating agents and coloring agents can also be incorporated into the mixture. Suitable binders include starch, gelatin, natural sugars such as glucose or beta-lactose, corn-sweeteners, natural and synthetic gums such as acacia, tragacanth or sodium alginate, carboxymethylcellulose, microcrystalline cellulose, sodium croscarmellose, polyethylene glycol, waxes and the like. Lubricants suitable for use in these dosage forms include sodium oleate, sodium stearate, magnesium stearate, sodium benzoate, sodium acetate, sodium chloride, and the like. Disintegrators suitable for use in these dosage forms include starch methyl cellulose, agar, bentonite, xanthan gum and the like.

Suitable pharmaceutically acceptable salts of the drugs described herein, and suitable for use in the method of the invention, are conventional non-toxic salts and can include a salt with a base or an acid addition salt such as a salt with an inorganic base, for example, an alkali metal salt (e.g., lithium salt, sodium salt, potassium salt, etc.), an alkaline earth metal salt (e.g., calcium salt, magnesium salt, etc.), an ammonium salt; a salt with an organic base, for example, an organic amine salt (e.g., triethylamine salt, pyridine salt, picoline salt, ethanolamine salt, triethanolamine salt, dicyclohexylamine salt, N1N′-dibenzylethylenediamine salt, etc.) etc.; an inorganic acid addition salt (e.g., hydrochloride, hydrobromide, sulfate, phosphate, etc.); an organic carboxylic or sulfonic acid addition salt (e.g., formate, acetate, trifluoroacetate, maleate, tartrate, methanesulfonate, benzenesulfonate, p-toluenesulfonate, etc.); a salt with a basic or acidic amino acid (e.g., arginine, aspartic acid, glutamic acid, etc.) and the like.

Various further aspects and embodiments of the present invention will be apparent to those skilled in the art in view of the present disclosure. All documents and database entries mentioned in this specification are incorporated herein by reference in their entirety. “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The drug can be administered in an oral form, for example, as tablets, capsules (each of which includes sustained release or timed release formulations), pills, powders, granules, elixirs, tinctures, suspensions, syrups, and emulsions, all well known to those of ordinary skill in the pharmaceutical arts. Likewise, the drug can be administered in intravenous (bolus or infusion), intraperitoneal, subcutaneous, or intramuscular form, well known to those of ordinary skill in the pharmaceutical arts.

The drug can be administered in the form of a depot injection or implant preparation that can be formulated in such a manner as to permit a sustained release of the active ingredient. The active ingredient can be compressed into pellets or small cylinders and implanted subcutaneously or intramuscularly as depot injections or implants. Implants can employ inert materials such as biodegradable polymers or synthetic silicones, for example, Silastic, silicone rubber or other polymers manufactured by the Dow-Corning Corporation.

The drug can also be administered in the form of liposome delivery systems, such as small unilamellar vesicles, large unilamellar vesicles and multilamellar vesicles. Liposomes can be formed from a variety of phospholipids, such as cholesterol, stearylamine, or phosphatidylcholines.

The drug can also be delivered by the use of monoclonal antibodies as individual carriers to which the compound molecules are coupled.

The drug can also be prepared with soluble polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues.

Furthermore, the drug can be prepared with biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates, and cross linked or amphipathic block copolymers of hydrogels. The dosage regimen utilizing the drug can be selected in accordance with a variety of factors including type, species, age, weight, sex and the type of cancer being treated; the severity (i.e., stage) of the cancer to be treated; the route of administration; the renal and hepatic function of the subject; and the particular compound or salt thereof employed. An ordinarily skilled physician or veterinarian can readily determine and prescribe the effective amount of the drug required to treat, for example, to prevent, inhibit (fully or partially) or arrest the progress of the disease.

Oral dosages of the drug, when used to treat the desired cancer can range between about 2 mg to about 6000 mg per day, such as from about 20 mg to about 6000 mg per day, such as from about 200 mg to about 6000 mg per day. For example, oral dosages can be about 2, about 20, about 200, about 400, about 800, about 1200, about 1600, about 2000, about 4000, about 5000 or about 6000 mg per day. It is understood that the total amount per day can be administered in a single dose or can be administered in multiple dosing such as twice, three or four times per day.

For example, a subject can receive between about 2 mg/day to about 2000 mg/day, for example, from about 20 to about 2000 mg/day, such as from about 200 to about 2000 mg/day, for example from about 400 mg/day to about 1200 mg/day. A suitably prepared medicament for once a day administration can thus contain between about 2 mg and about 2000 mg, such as from about 20 mg to about 2000 mg, such as from about 200 mg to about 1200 mg, such as from about 400 mg/day to about 1200 mg/day. The drug can be administered in a single dose or in divided doses of two, three, or four times daily. For administration twice a day, a suitably prepared medicament would therefore contain half of the needed daily dose.

Intravenously or subcutaneously, the subject would receive the drug in quantities sufficient to deliver between about 3-1500 mg/m2 per day, for example, about 3, 30, 60, 90, 180, 300, 600, 900, 1000, 1200, or 1500 mg/m2 per day. Such quantities can be administered in a number of suitable ways, e.g., large volumes of low concentrations of drug during one extended period of time or several times a day. The quantities can be administered for one or more consecutive days, intermittent days, or a combination thereof per week (7 day period). Alternatively, low volumes of high concentrations of drug during a short period of time, e.g., once a day for one or more days either consecutively, intermittently, or a combination thereof per week (7 day period). For example, a dose of 300 mg/m2 per day can be administered for 5 consecutive days for a total of 1500 mg/m2 per treatment. In another dosing regimen, the number of consecutive days can also be 5, with treatment lasting for 2 or 3 consecutive weeks for a total of 3000 mg/m2 and 4500 mg/m2 total treatment.

Typically, an intravenous formulation can be prepared which contains a concentration of drug of from about 1.0 mg/mL to about 10 mg/mL, e.g., 2.0 mg/mL, 3.0 mg/mL, 4.0 mg/mL, 5.0 mg/mL, 6.0 mg/mL, 7.0 mg/mL, 8.0 mg/mL, 9.0 mg/mL, or 10 mg/mL, and administered in amounts to achieve the doses described above. In one example, a sufficient volume of intravenous formulation can be administered to a subject in a day such that the total dose for the day is between about 300 and about 1200 mg/m2.

In a preferred embodiment, 1000 mg/m2 of PXD-101 is administered intravenously once daily by 30-minute infusion every 24 hours for at least five consecutive days.

In one embodiment, PXD-101 is administered in a total daily dose of up to 1500 mg/m2. In one embodiment, PXD-101 is administered intravenously in a total daily dose of 1000 mg/m2, or 1400 mg/m2 or 1500 mg/m2, for example, once daily, continuously (every day), or intermittently. In one embodiment, PXD-101 is administered every day on days 1 to 5 every three weeks.

Glucuronic acid, L-lactic acid, acetic acid, citric acid, or any pharmaceutically acceptable acid/conjugate base with reasonable buffering capacity in the pH range acceptable for intravenous administration of the drug can be used as buffers. Sodium chloride solution wherein the pH has been adjusted to the desired range with either acid or base, for example, hydrochloric acid or sodium hydroxide, can also be employed. Typically, a pH range for the intravenous formulation can be in the range of from about 5 to about 12. A preferred pH range for intravenous formulation wherein the drug has a hydroxamic acid moiety (e.g., as in PXD-101), can be about 9 to about 12. Consideration should be given to the solubility and chemical compatibility of the drug in choosing an appropriate excipient.

Subcutaneous formulations, preferably prepared according to procedures well known in the art at a pH in the range between about 5 and about 12, also include suitable buffers and isotonicity agents. They can be formulated to deliver a daily dose of drug in one or more daily subcutaneous administrations, e.g., one, two or three times each day. The choice of appropriate buffer and pH of a formulation, depending on solubility of the drug to be administered, is readily made by a person having ordinary skill in the art. Sodium chloride solution wherein the pH has been adjusted to the desired range with either acid or base, for example, hydrochloric acid or sodium hydroxide, can also be employed in the subcutaneous formulation. Typically, a pH range for the subcutaneous formulation can be in the range of from about 5 to about 12. A preferred pH range for subcutaneous formulation wherein the drug has a hydroxamic acid moiety is about 9 to about 12. Consideration should be given to the solubility and chemical compatibility of the drug in choosing an appropriate excipient.

The drug can also be administered in intranasal form via topical use of suitable intranasal vehicles, or via transdermal routes, using those forms of transdermal skin patches well known to those of ordinary skill in that art. To be administered in the form of a transdermal delivery system, the administration will likely be continuous rather than intermittent throughout the dosage regime.

The further chemotherapeutic agent (or agents, if more than one is employed) may be administered using conventional methods and protocols well known to those of skill in the art. For example, a typical dosage rate for 5-fluorouracil (5-FU) is 750-1000 mg/m2 in a 24 hour period, administered for 4-5 days every 3 weeks. A typical dose rate for capecitabine is 1000 to 1250 mg/m2 orally, when administered twice daily on days 1 to 14 of every 3rd week.

In another embodiment of the disclosure, an article of manufacture containing materials useful for the treatment of the diseases or disorders described above is provided. The article of manufacture may comprise a container and a label or package insert on or associated with the container. Suitable containers include, for example, bottles, vials or syringes. The containers may be formed from a variety of materials such as glass or plastic. The container holds a composition that may be effective for treating the condition and may have a sterile access port (e.g., the container may be an intravenous solution bag or a vial having a stopper pierceable by a hypodermic injection needle). At least two active agents in the composition may be one or more methyltransferase inhibitors, such as Methotrexate and one or more tyrosine kinase inhibitors. The label or package insert may indicate that the composition may be used for treating the condition of choice, such as cancer.

Moreover, the article of manufacture may comprise (a) a first container with a composition contained therein, wherein the composition comprises one or more methyltransferase inhibitors, such as Methotrexate, and (b) a second container with a composition contained therein, wherein the composition comprises one or more receptor tyrosine kinase inhibitors. The article of manufacture in this embodiment of the disclosure may further comprise a package insert indicating that the first and second compositions can be used in combination to treat a disease or disorder including, for example, cancer. Additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

### Example 1

### Materials and Methods

The following materials and methods were employed throughout the Examples. However, it is to be understood that modification of these materials and methods in addition to the use of alternative materials and methods are contemplated within the scope of this disclosure.

### Cell Culture

All cell lines were purchased from American Type Culture Collection (Manassas, Va.) except COR-L23 (Sigma-Aldrich, St. Louis, Mo.). A549 cells were cultured in IDMEM medium; Calu6 cells were cultured in EMEM medium; H460, H2122, H358, H1792, H1734, H661, H2126, H1993,H1299, H1395, H23, H1975, H727, H2009, COR-L23 and Calu3 cells were cultured in RPMI. All media were purchased from Mediatech and supplemented with 10% Fetal Bovine Serum (Life Technologies, Carlsbad, Calif.) and antibiotics (Mediatech, Manassas, Va.). Cells were cultured in a 37° C., 5% CO2 humidified environment.

### Cell Proliferation Assay

Cells were seeded into 96 well culture plates and left to attach overnight. Overnight culture media was removed and cells were treated with 0-10 uM of either Methotrexate (Sigma-Aldrich, St. Louis, Mo.) or Pemetrexed (LC Laboratories, Woburn, Mass.) or the appropriate vehicle in 100 uL of treatment media. Cells were incubated for 72 hours. Cyquant® Direct Cell Proliferation Assay (Life Technologies, Carlsbad, Calif.) was utilized to assess cell proliferation as per the manufacturer's instructions. Plates were read on a Bio-Tek Synergy 2 plate reader.

### RNA Isolation

Following treatment, total RNA was isolated utilizing the miRNEasy kit (Qiagen Inc, Valencia, Calif.) according to the manufacturer's instructions. Quality and concentration were assessed using a Nanodrop ND-1000 Spectrophotometer (Thermo Fisher, Waltham, Mass.).

### Gene Expression

Gene Expression levels were assessed on isolated RNA by real time RT-PCR analysis using the High Capacity cDNA Reverse Transcription kit and TaqMan Gene Expression Master Mix (both Life Technologies, Carlsbad, Calif.). Relative expression was calculated using the ΔΔCT method on an ABI 7900HT Fast Real Time PCR System (Life Technologies, Carlsbad, Calif.). Taqman Gene Expression assays from Life Technologies, (Carlsbad, Calif.) included: KRAS (Hs000364284_g1), DHFR (Hs0075828_s1), Human β-Actin (ABI#4326315E) and Human β-2-Microglobulin (normalization control).

### Western Blot Analysis

Following treatment, cells were harvested for lysates using Cell Lysis Buffer (Cell Signaling Technologies, Danvers, Mass.) supplemented with 1 mM Phenylmethanesulfonyl fluoride (PMSF) and a 1:10 dilution of PI Cocktail (both from Sigma-Aldrich, St. Louis, Mo.) as well as a 1:200 dilution of each Phosphoguard A and Phosphoguard B (Therapak, Atlanta, Ga.). 25-30 ug of total cell lysate was resolved on a 4-15% TGX acrylamide gel (Bio-Rad Laboratories, Hercules, Calif.) for 35 minutes at 200 volts and transferred to Imobilon®-FL (EMD Millipore, Billerica, Mass.) membrane in Tris/Glycine/Methanol transfer buffer for 2 hours at 50 volts. Immunoblotting was performed with primary antibody overnight at 4° C. Thymidylate Synthase (TS) Antibody from Cell Signaling Technologies (Danvers, Mass.). KRAS antibody was from Abgent Inc. (San Diego, Calif.) and DHFR antibody was from Sigma-Aldrich (St. Louis, Mo.). Beta-Actin (Sigma-Aldrich, St. Louis, Mo.) was used as a loading control. Secondary antibodies: anti Mouse IRDye800 and anti Rabbit IRDye680 were from LI-COR Biosciences (Lincoln, Nebr.). Membranes were scanned on a LICOR Odyssey Infrared Imager.

### KRAS Over Expression

K-RAS2 2XMYC-tagged vector (cloneID: RASK20MN00) (a gift from Dr. David Solit's lab) or pcDNA3.1+ vector alone (Life Technologies, Carlsbad, Calif.) was transfected into the cells using Lipofectamine 200011 (Life Technologies, Carlsbad, Calif.) as per the manufacturer's instructions. Cells were harvested after 48 hours for either protein lysates or gene expression analysis as described above.

KRAS siRNA Knockdown

KRAS levels were knocked down using siRNA against KRAS, Dharmacon ON-TARGET SMARTpool, Human KRAS; (Thermo Fisher, Waltham, Mass.) or a Scrambled Control (Dharmacon ON-TARGETplus Nontargeting siRNA#1; (Thermo Fisher, Waltham, Mass.) with DharmaFECT1 siRNA Transfection Reagent (Thermo Fisher, Waltham, Mass.) as per the manufacturer's instructions. Cells were harvested after 48 hours for either protein lysates or gene expression analysis as described above.

### Cell Cycle Analysis

Following treatment cells were lifted from the plate, washed with cold Phosphate Buffered Saline (PBS) and fixed in absolute ethanol overnight at −20° C. Cells were then washed twice in cold PBS and resuspended in a 200 ug/mL Propidium Iodide (Life Technologies, Carlsbad, Calif.) Buffer containing 0.1% (v/v) Triton x-100 (Sigma-Aldrich, St. Louis, Mo.), 0.2 mg/mL RNAse A (Sigma-Aldrich, St. Louis, Mo.) in PBS. Cells were incubated for 30 minutes at room temperature in the dark and transferred to ice until analyzed. Cells were analyzed on a FACSCantoll Flow Cytometer (BD Biosciences, San Jose, Calif.) with FCS Express Multicycle Software (Phoenix Flow Systems, San Diego, Calif.).

### KRAS Mutation Testing

KRAS exon 2 was amplified from DNA extracted from patient DNA and sequenced using an ABI model 3730 capillary gel sequencer. Mutations were identified by visual inspection of the resulting chromatograms and automated scanning using Mutation Surveyor v3.24.

### Example 2

### One Carbon Pool by Folate Pathway is Upregulated in KRASmut Versus KRASwt NSCLC Cells

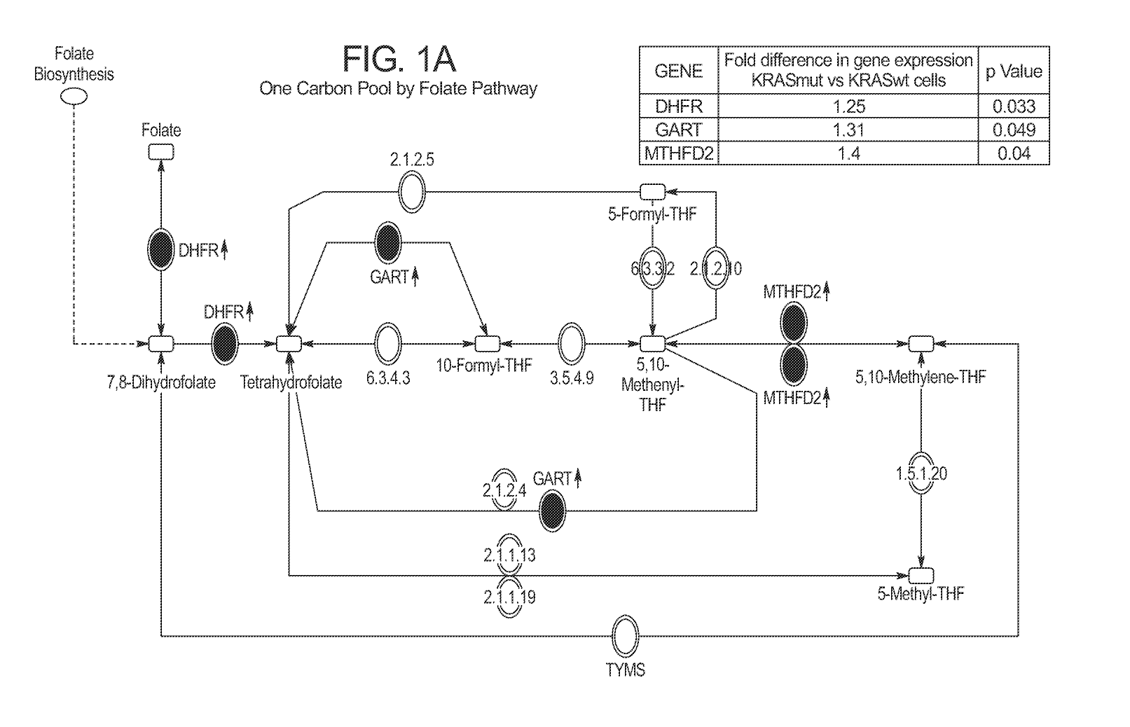

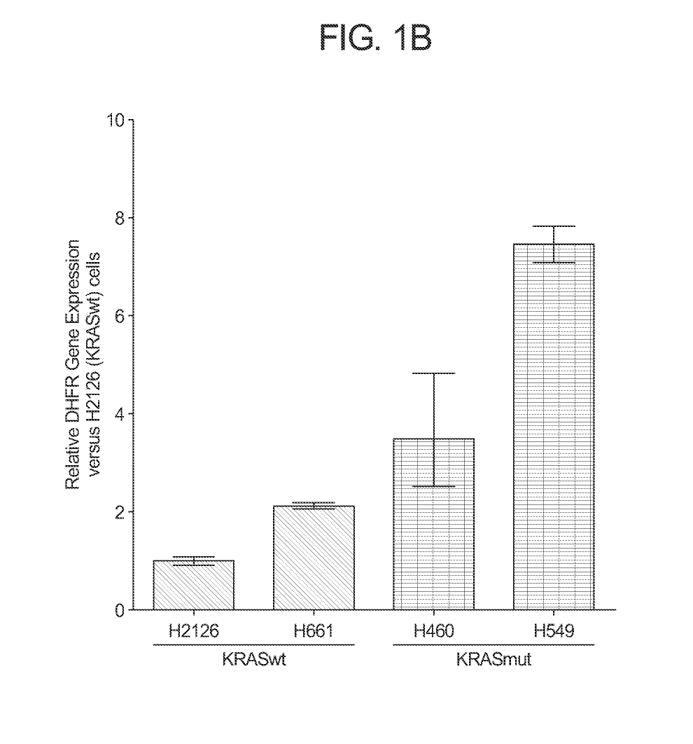

Microarray gene expression data for NSCLC cell lines from a NCI-60 dataset were compiled and grouped according to KRAS mutation status. Genes showing significant differences (p<0.05) in expression between KRASmut and KRASwt cells were used for Ingenuity Pathway Analysis which assigns genes into biological pathways and networks. Analysis indicated several biological pathways that differed between these two groups. Interestingly, the one carbon pool by folate pathway was moderately but significantly higher in KRASmut cells (FIG. 1A). One carbon pool by folate pathway describes the activity of enzymes involved in metabolism of folate and transfer of one carbon units needed for molecular biosynthesis in cells.

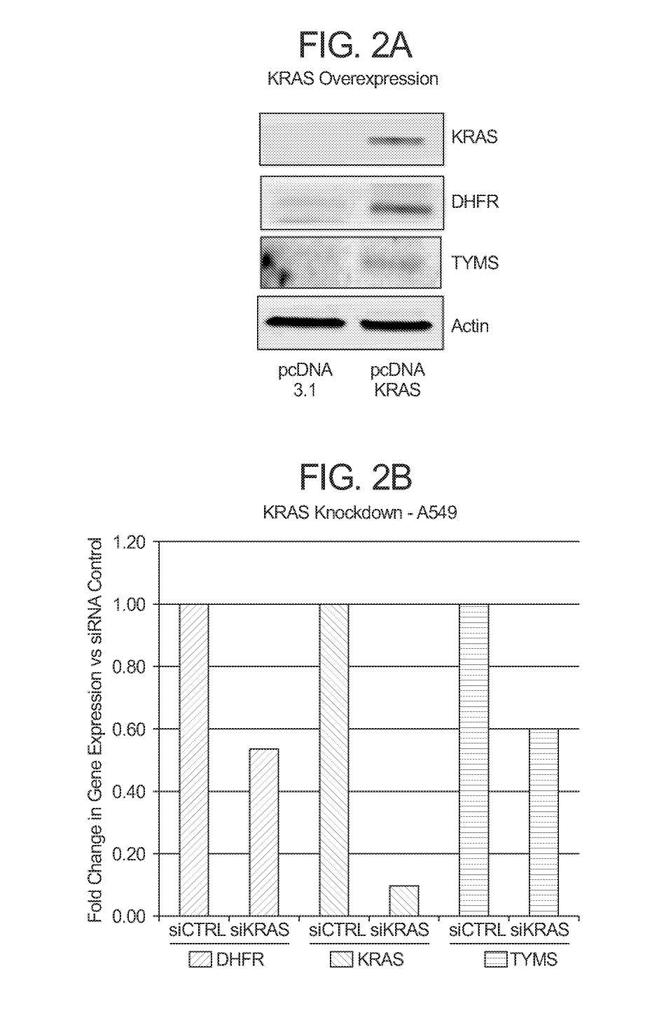

In order to validate these findings, RT-PCR for DHFR (a gene which showed significant differences between these groups in microarray data) was performed on representative KRASmut and KRASwt cells. KRASmut representative cells had significantly higher expression of DHFR versus their wildtype counterparts by RT-PCR analysis (FIG. 1B). In order to investigate this relationship further, A549 (KRASmut) cells were transfected with siRNA targeting KRAS (siKRAS) or control siRNA (siCTRL). Knockdown of KRAS significantly decreased DHFR and TYMS expression as assessed by qRT-PCR (FIG. 2A). Conversely, transfection of A549 cells with a KRAS expression vector upregulated both TYMS and DHFR protein expression (FIG. 2B).

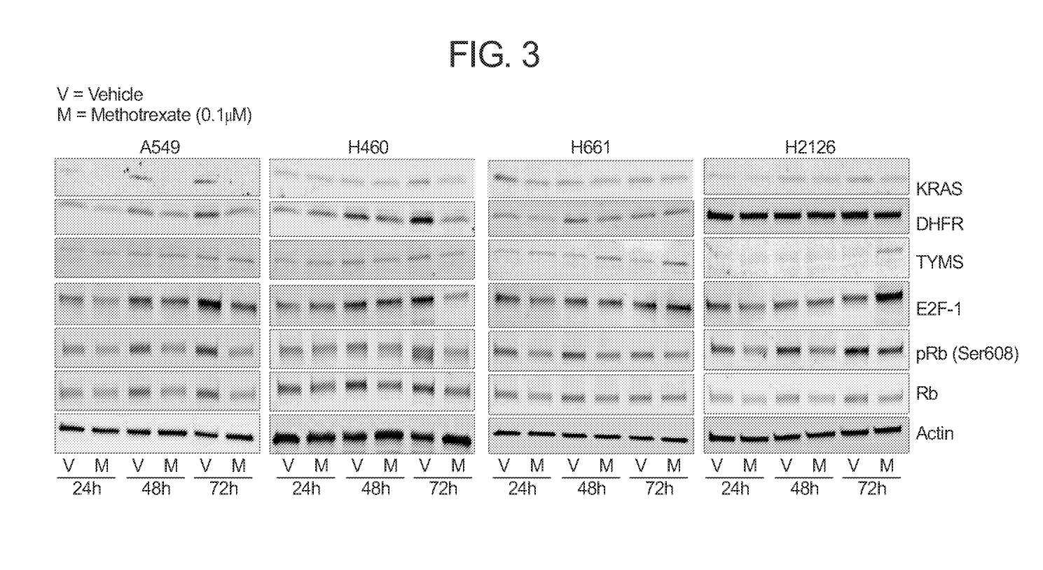

KRAS was also tested for its ability to regulate gene expression in KRAS mutant NSCLC cell lines. In an exemplary method, A549 (KRAS mutant) cells were transfected with an expression vector containing KRAS ORF or an empty vector control (pcDNA3.1). Next, lysates were collected 48 hours after transfection and were subsequently analyzed by immunoblotting using antibodies specific to KRAS, DHFR, E2F-1, and pRb (Ser608). β-Actin was used as loading control. In the tested cells, KRAS drove the expression of DHFR, E2F-1 and the phosphorylation of Rb (see, FIG. 3). Additionally, A549 cells were transfected with siRNA targeting KRAS (siKRAS) or non-targeting control (siCTRL). Next, RNA was harvested at 72 hours after transfection and subsequently analyzed by qPCR using primers specific to DHFR and KRAS on an ABI 7900HT RT-PCR system. Gene expression was normalized to internal β-2-Microglobulin control and expressed as fold change versus siCTRL. DHFR gene expression decreased in those cells treated with siKRAS indicating that KRAS drives expression of DHFR (see, FIG. 3).

Collectively, these data suggest a relationship between mutant KRAS and folate driven pathways in NSCLC cells.

### Example 3

### Sensitivity of KRAS Mutant NSCLC Cell Lines to Antifolates

Cells with a different status of KRAS (KRAS mutant or KRAS wild type) were tested for their sensitivity to an antifolate such as Methotrexate or Pemetrexed. In an exemplary method, KRAS mutant (H1734, H460, Calu6, H358, H1792, H2122, A549 and H23) and KRAS wild-type (H1299, H1993, H1395, Calu3, H1975, H661 and H2126) NSCLC cell lines (see, Table 1) were treated with multiple doses (0-10 μM) of Methotrexate or Pemetrexed for 72 hours and were subsequently assayed for proliferation using the Cyquant Direct (Invitrogen) proliferation assay. Mutation and amplification status of each cell line was determined from the Sanger Catalogue of Somatic Mutations in Cancer database (COSMIC). Cells harboring greater than 7 copies of KRAS were considered for the purposes of this study to be amplified for KRAS as specified in the criteria of the COSMIC copy number database.

TABLE 1

| Cell | KRAS Copy | | |
|---|---|---|---|
| Line | Number | KRAS | NRAS |
| A549 | 3 | G12S (c.34G > A) | WT |
| H460 | 2 | Q61H (c.183A > T) | WT |
| H2122 | 2 | G12C (c.34G > T) | WT |
| H358 | 6 | G12C (c.34G > T) | WT |
| H1792 | 2 | G12C (c.34G > T) | WT |
| H1734 | 5 | G13C (c.37G > T) | WT |
| Calu6 | 5 | Q61K (c.180_181TC > | WT |
| | | CA) | |
| H23 | 4 | G12C (c.34G > T) | WT |
| H2009 | 9 | G12A (c.35G > C) | WT |
| | | (amplified) | |
| H727 | 8 | G12V (c.35G > T) | WT |
| | | (amplified) | |
| COR-L23 | 14 | G12V (c.35G > T) | WT |
| | | (amplified) | |
| H1395 | 1 | WT | WT |
| H1299 | 1 | WT | Q61K (c.181C > A) |
| H1975 | 2 | WT | WT |
| Calu3 | 1 | WT | WT |
| H2126 | 3 | WT | WT |
| H1993 | 1 | WT | WT |
| H661 | 10 | WT (amplified) | WT |

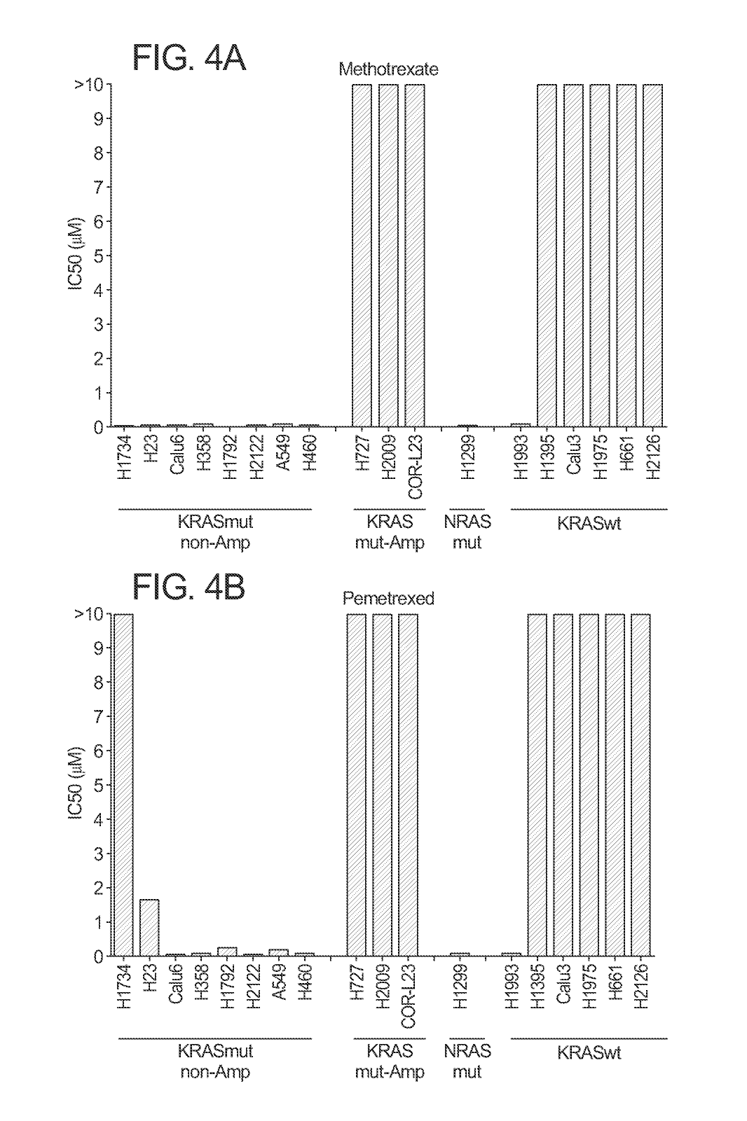

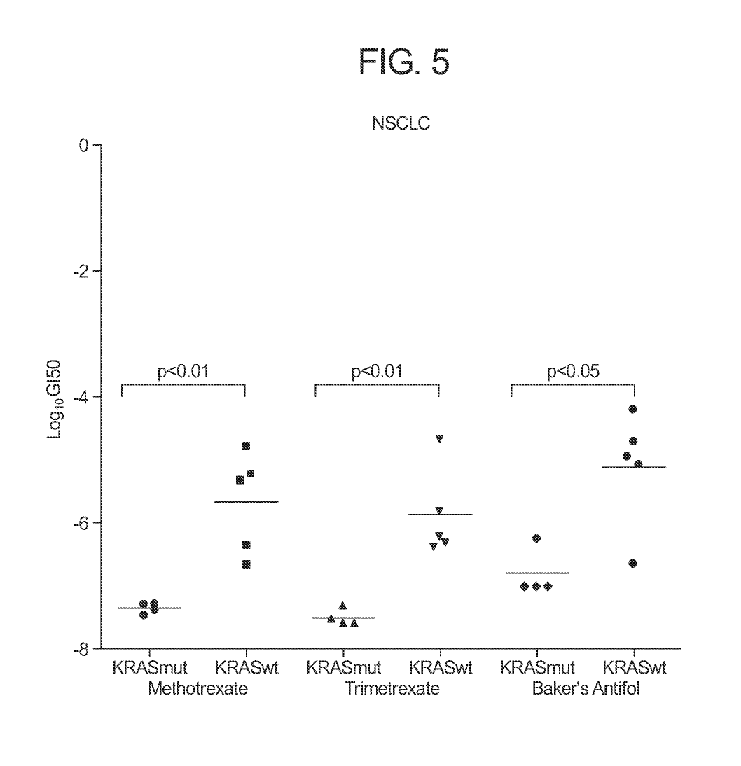

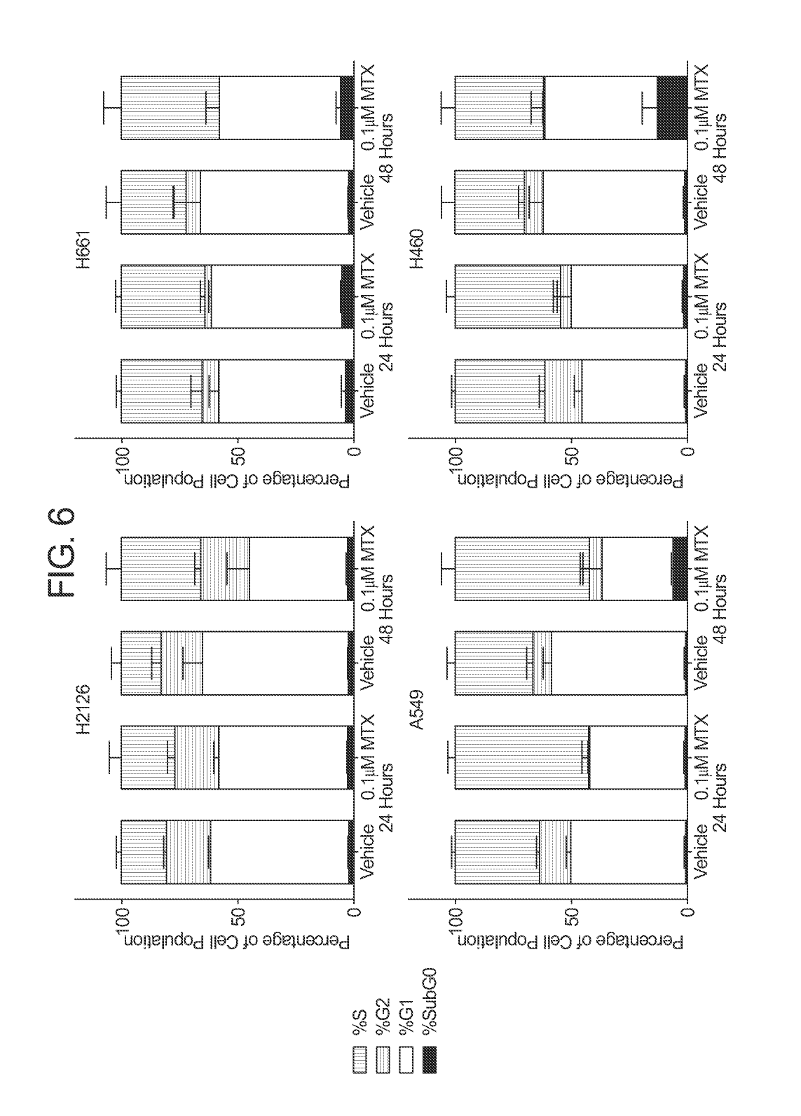

KRASwt cell lines were relatively resistant to MTX and PEM with average IC50 values greater than 10 μM (FIGS. 4A & 4B). Conversely, KRASmut non-amplified cell lines were significantly more sensitive to MTX and PEM with average IC50 values below 0.3 μM. Furthermore, an NRAS mutant (Q61K) cell line (H1299) was sensitive to both MTX and PEM. Interestingly, KRASmut amplified cells did not respond to MTX or PEM (IC50>10 μM). To evaluate this further in an independent dataset, the NCI Developmental Therapeutics Program cancer drug screen database was interrogated to identify compounds that selectively inhibit the proliferation of KRASmut NSCLC cell lines. Data from this study indicated that MTX and other antifolates (Trimetrexate & Soluble Baker's Antifol) have significantly higher GI50 values in KRASmut versus KRASwt NSCLC cells (FIG. 5). Finally, examination of the effects of MTX on cell cycle progression in KRASmut versus KRASwt cells indicated a more prominent S phase arrest and greater accumulation in sub-G populations in KRASmut versus KRASwt cells (FIG. 6).

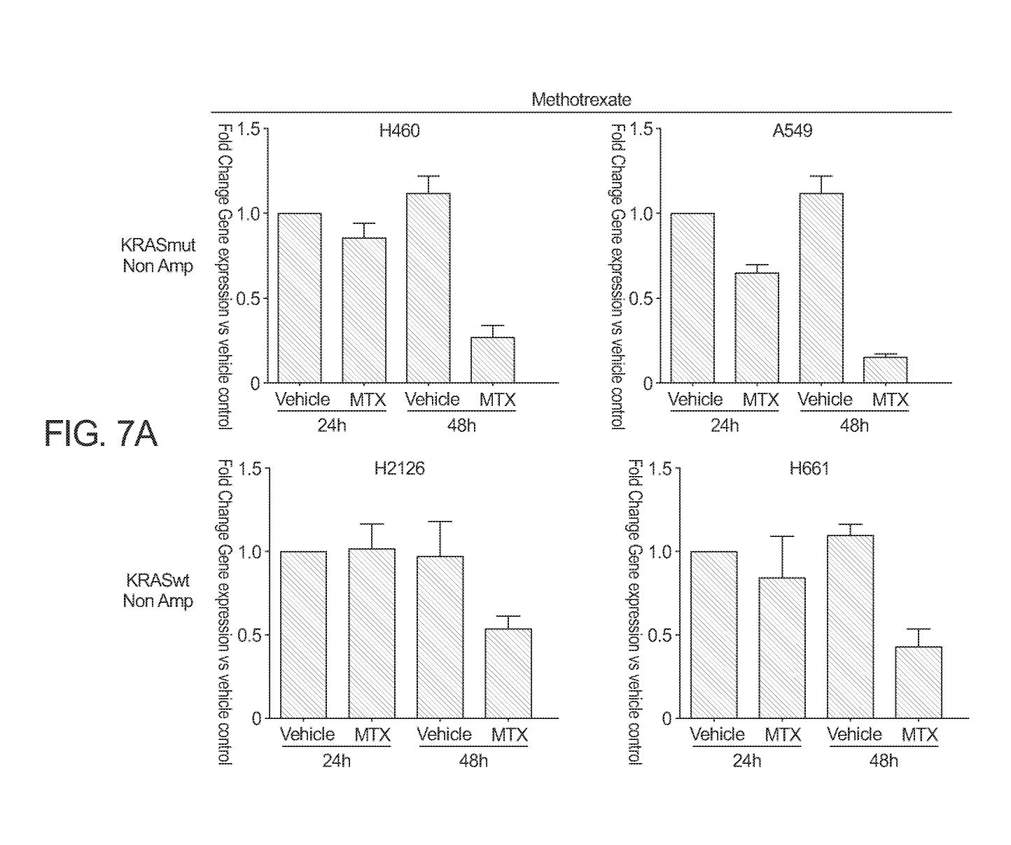

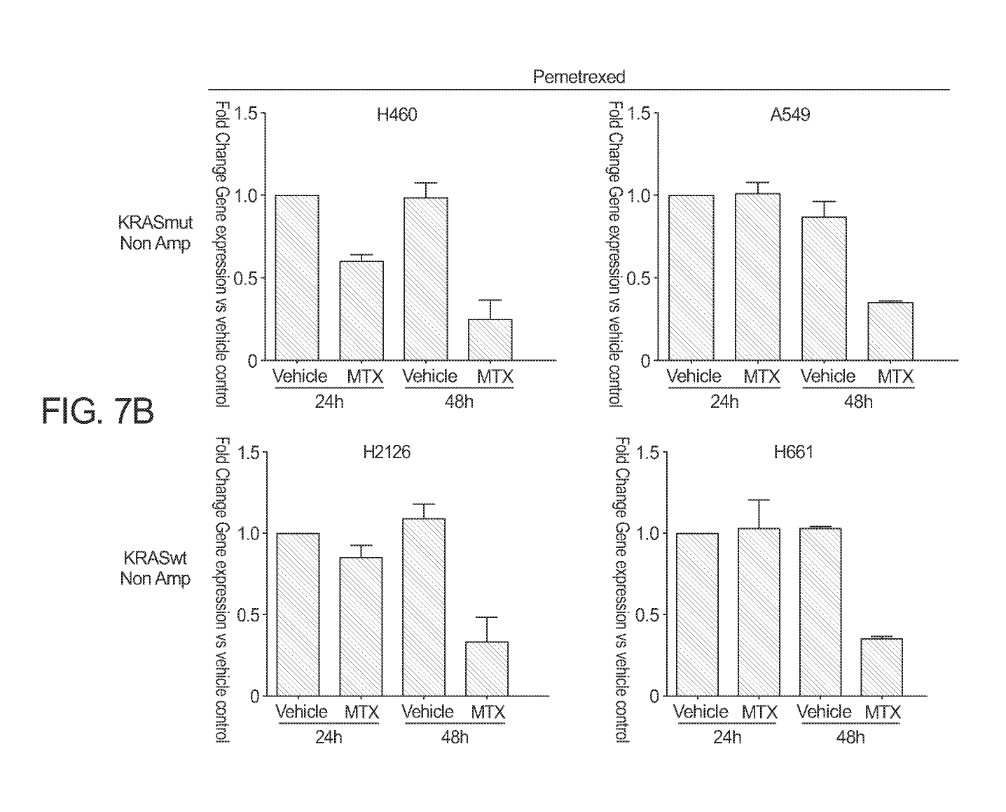

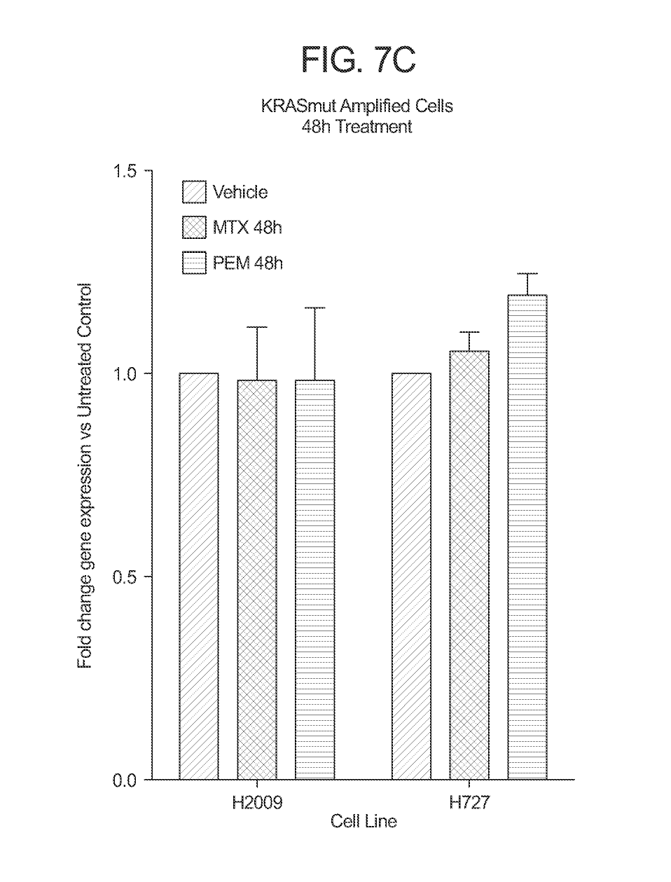

KRAS gene expression was next examined in the presence of antifolates. Representative KRASmut non-amplified, KRASwt and KRASmut amplified NSCLC cell lines were treated with MTX (0.1 μM), PEM (0.1 μM) or vehicle (DMSO) as control. RNA was harvested at 24 h and 48 h after treatment and was analyzed by qRT-PCR using primers specific to KRAS. Both PEM and MTX significantly reduced KRAS mRNA expression level in KRASmut non-amplified and KRASwt cell lines (FIG. 7A, 7B). KRAS expression was not altered by antifolate treatment in KRASmut amplified cells (FIG. 7C). Antifolates did not change the expression of unrelated genes such as β-Actin in our studies indicating that these decreases in KRAS gene expression do not reflect a shutdown of global cellular transcriptional activity (data not shown). Thus decreases in KRAS gene expression could account for the higher efficacy of antifolates in KRASmut cells since these cells are typically more dependent on the presence of active KRAS for survival and growth than wildtype cells.

### Example 4

### Antifolate Mediated Upregulation of miRNAs that Target KRAS in KRAS Mutant NSCLC Cells

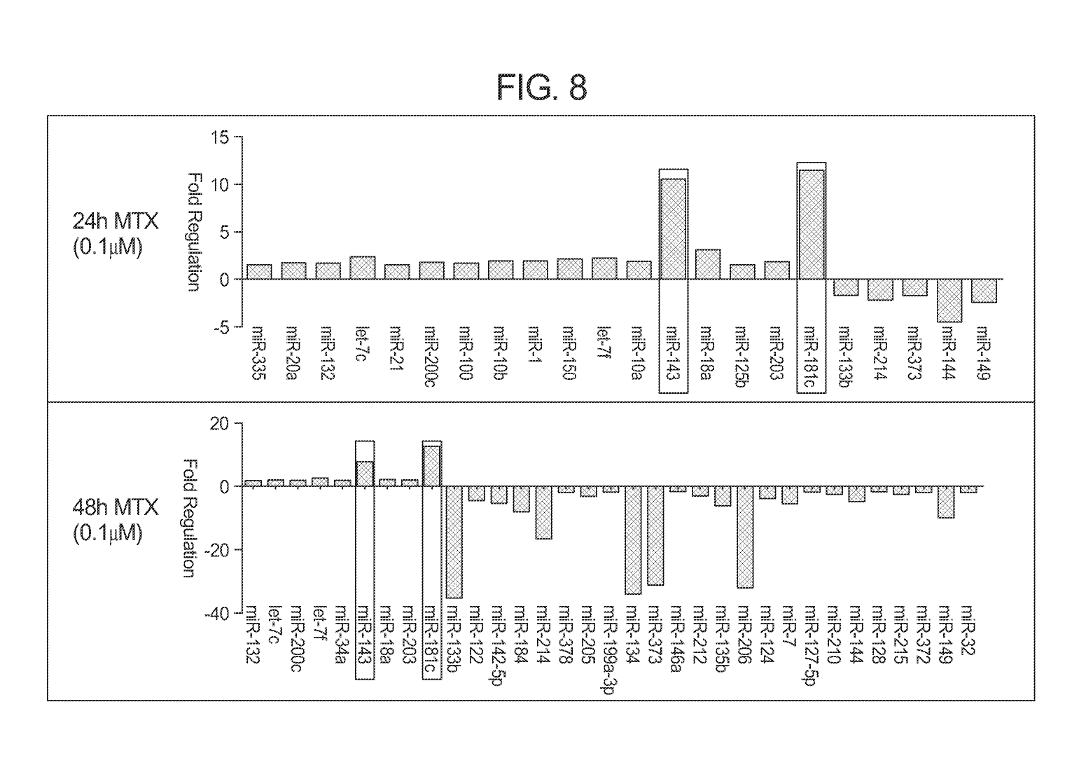

Cells with a different status of KRAS (KRAS mutant or KRAS wild type) were tested for their sensitivity to a drug such as an antifolate including, for example, a DHFR inhibitor. In an exemplary method, representative KRAS mutant (H460) NSCLC cell line was treated with Methotrexate (0.1 μM) or DMSO as control. Next, RNA was harvested at 24 hours and 48 hours after treatment and subsequently analyzed using cancer microRNA PCR arrays (Qiagen) on an ABI 7900HT RT-PCR system. Gene expression was then normalized to housekeeping gene controls and expressed as fold regulation versus vehicle Control (24 hours). Methotrexate strongly upregulated the expression of miR-143 and miR-181 in the KRAS mutant NSCLC cells (FIG. 8).

### Example 5

### Sensitivity of KRAS Mutant Non-Amplified, KRAS Mutant Amplified, and KRAS Wild-Type Cells to Treatment with an Antifolate

Cells with a different status of KRAS (KRAS mutant or KRAS wild type) may be tested for their sensitivity to a drug such as an antifolate including, for example, a DHFR inhibitor.

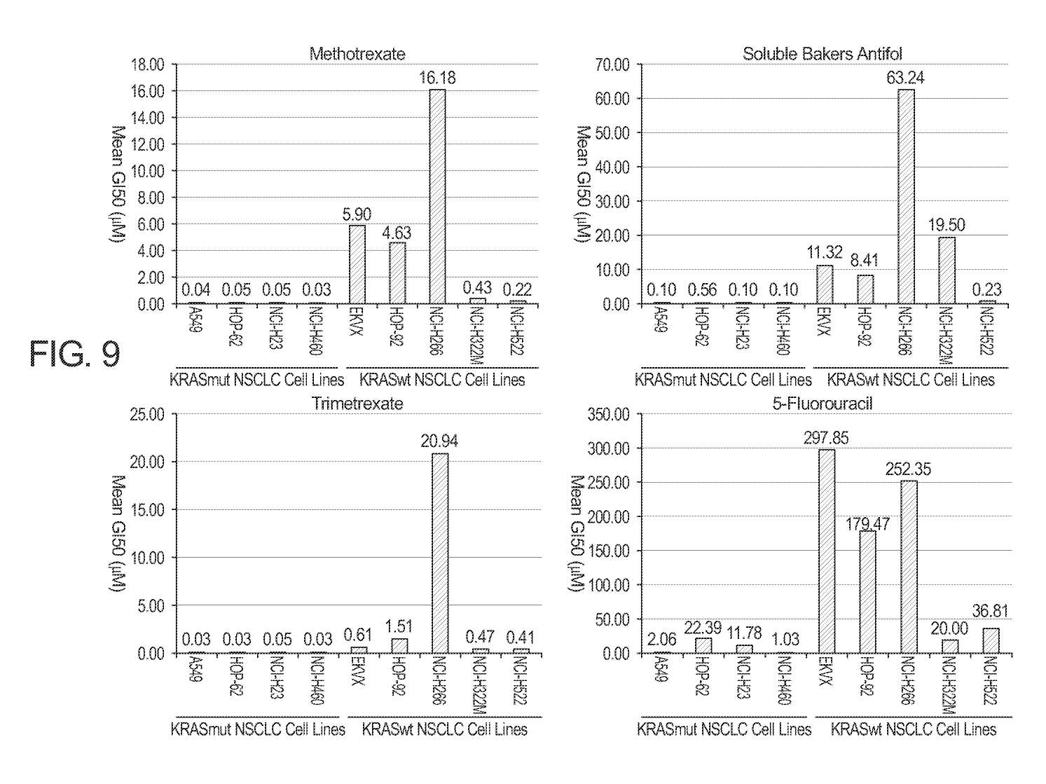

In an exemplary method, the NCI Developmental Therapeutics Program cancer drug screen database was also interrogated for association between KRAS mutation status and drug efficacy in NCI60 NSCLC cell lines. This database compiles results from multiple experiments in which the NCI-60 bank of cell lines were treated with 5 doses of each drug and assayed for proliferation 48 hours later. Analysis of this data demonstrates lower GI₅₀ values for antifolates in KRAS mutant versus KRAS wild-type NSCLC cell lines. As such, this database revealed increased efficacy of antifolates in KRAS mutant versus KRAS wild-type NCI-60 NSCLC cell lines (FIG. 9). Additionally, a similar specificity was revealed for other anti-folate therapies in the NCI cell screen.

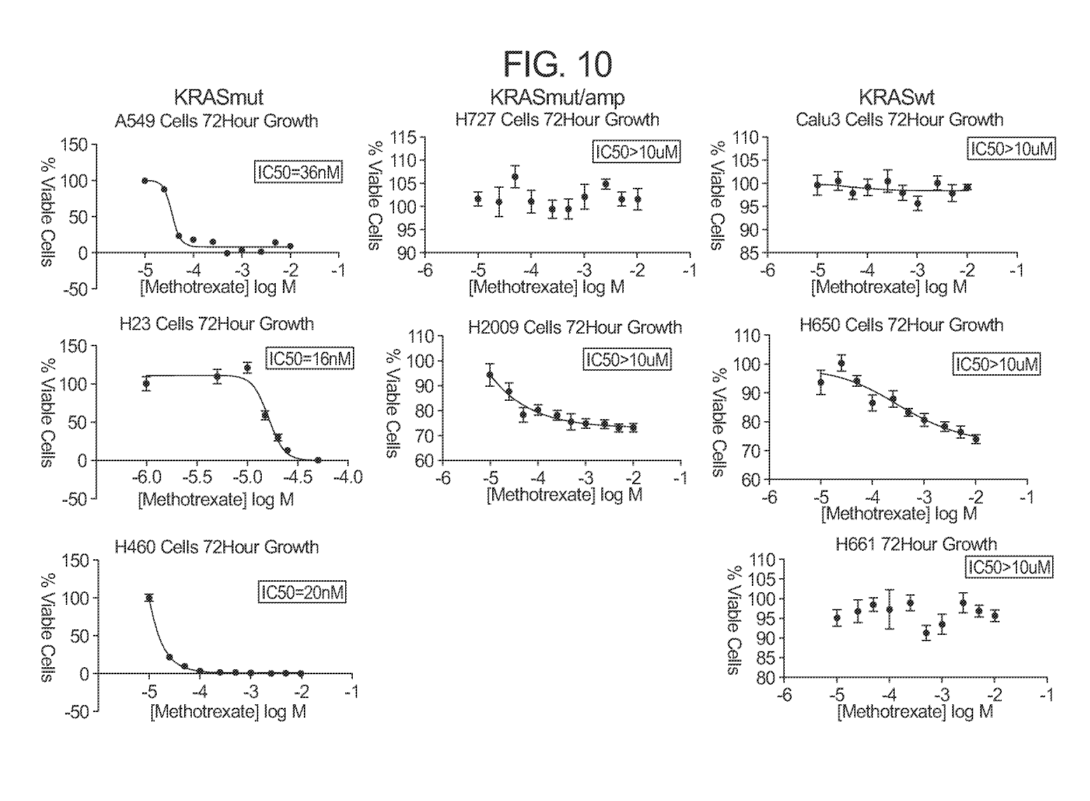

Additionally, a variety of NSCLC cell lines that were KRAS mutant (A549, NCI-H460 & NCI-H23), KRAS mutant/amplified (NCI-H727 & NCI-H2009) and KRAS wild-type (Calu-3, NCI-H650 & NC-H661) NSCLC cells were plated in 96 well plates treated and treated 24 hours later with multiple concentrations of Methotrexate (0-10 μM). After an additional 72 hours cells were assayed for proliferation using the Invitrogen Cyquant Direct™ proliferation assay. IC₅₀ (inhibitory concentration that kills 50% of cells) was determined using graphpad software. Cells were treated in triplicate and cell numbers were calculated as percent untreated control. KRAS mutant (A549, NCI-H460 & NCI-H23) were sensitive to Methotrexate while KRAS mutant/amplified (NCI-H727 & NCI-H2009) cells and KRAS wild-type (Calu-3, NCI-H650 & NC-H661) cells were not sensitive to Methotrexate (FIG. 10).

Collectively, these studies highlight increased sensitivity to an antifolate in KRAS mutant non-amplified NSCLC cells. Without being bound to a theory of the invention, it is believed that mutant KRAS drives expression and release of E2F-1 which may in turn lead to increased expression of DHFR/TS and potential dependency on these pathways.

### Example 6

### KRAS Expression Levels in KRAS Mutant Amplified and KRAS Mutant Non-Amplified NSCLC Cell Lines

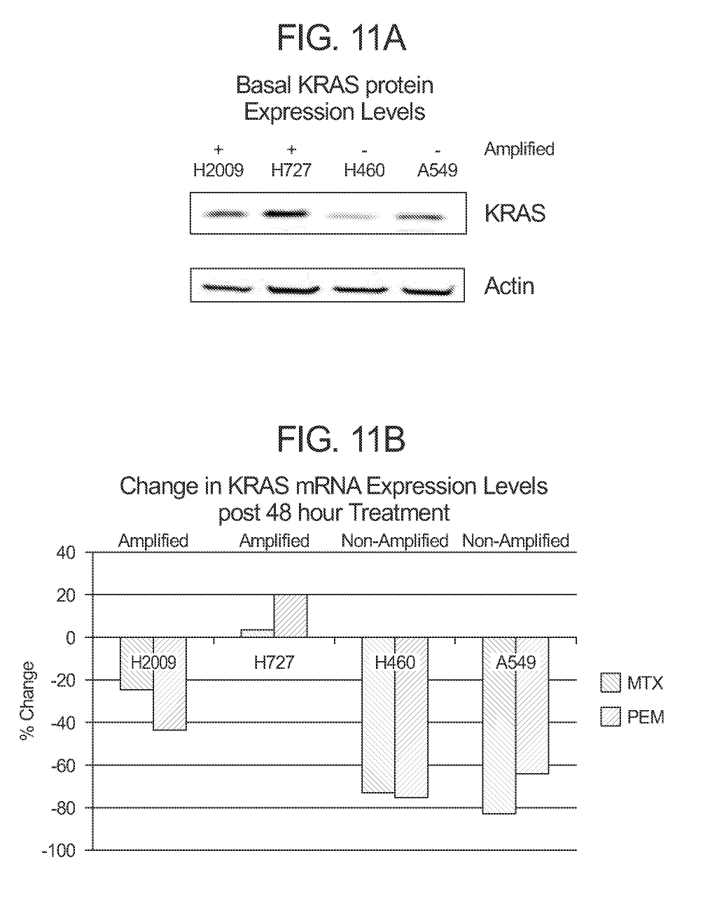

KRAS expression levels were determined in KRAS mutant NSCLC cell lines that harbor an amplification of the KRAS gene or do not harbor an amplification of the KRAS gene. In an exemplary method, H2009, H727, H460 and A549 cells were incubated for 24 hours and subsequently lysed to create protein samples. Next, protein concentrations were equalized and lysates were immunoblotted for KRAS and actin as a loading control. Basal expression of KRAS was higher in KRAS mutant amplified (H2009 and H727) versus KRAS mutant non-amplified (H460 and A549) cells (see, FIG. 11A). KRAS expression levels were then tested in these same cells after treatment of the cells with Methotrexate (MTX) or Pemetrexed (PEM). Briefly, H2009, H727, H460 and A549 cells were incubated for 24 hours prior to treatment with 0.1 uM of MTX or PEM. Next, 48 hours later cells were lysed, RNA was prepared and subsequently analyzed by qRT-PCR using primers specific to KRAS. Methotrexate and Pemetrexed reduced KRAS mRNA expression to a greater extent in KRAS mutant non amplified cells versus KRAS mutant amplified cells (see, FIG. 11B).

### Example 7

### Responsiveness of KRAS Mutant Tumors to Methotrexate Treatment in Vivo

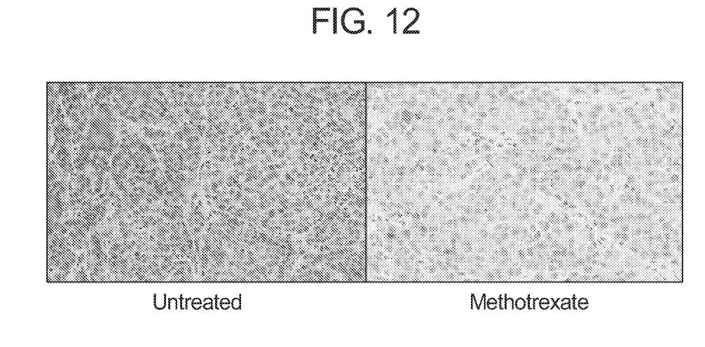

Tumors with a different status of KRAS (KRAS mutant or KRAS wild type) were tested in vivo for their sensitivity to a drug. In an exemplary method, H460 cells (KRASmut non-amplified) determined to be sensitive to Methotrexate were implanted in mice and grown to approximately 500 mg before treatment with 130 mg/kg Methotrexate Q4Dx3 IP. Tumors were then harvested 10 days after treatment, fixed in formalin and stained for cleaved caspase-3. Next, bright-field pictures were taken at 40× (see, FIG. 12). Tumors with KRAS mutant cells were shown to be sensitive (e.g., responsive) to Methotrexate.

### Example 8

### Clinical Case Reports

Pemetrexed is approved for NSCLC treatment. The following section describes two clinical case reports detailing dramatic and durable responses to pemetrexed in KRAS mutant patients.

### Case 1

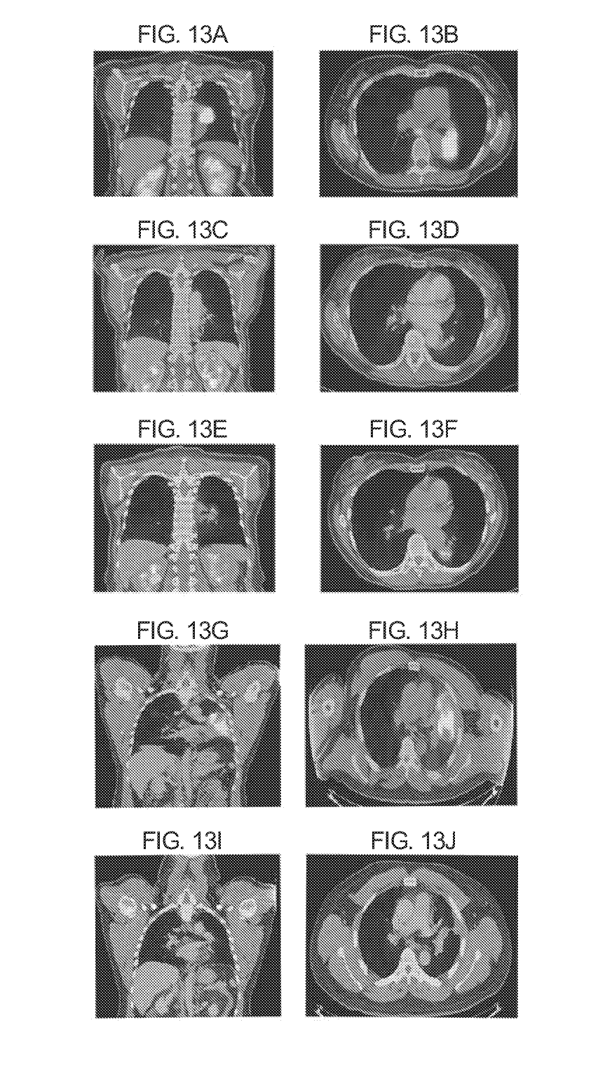

A 52 year old Caucasian woman with an ongoing smoking history presented in October 2008 with persistent headaches. A brain MRI revealed 2 left sided intracerebral lesions. A CT scan of the chest and abdomen revealed a dominant left lower lobe mass with associated subcarinal, precarinal and paratracheal lymphadenopathy. Bronchoscopy revealed a partially obstructing left lower lobe lesion with biopsies demonstrating a poorly differentiated Thyroid Transcription Factor-1 (TTF-1) positive adenocarcinoma of the lung. The brain lesions were resected, showing pathology consistent with the lung primary. A staging PET/CT scan revealed multiple hypermetabolic foci involving lymph nodes and a dominant left perihilar mass measuring 2.5×4 cm (FIG. 13A, 13B). Molecular testing revealed the tumor as bearing a G12C mutation in KRAS. Patient tissue was not available for FISH testing. The patient commenced chemotherapy with carboplatin and pemetrexed. The first CT scan performed after 2 cycles of treatment showed a reduction in the longest diameter of the left hilar mass to 2.2 cm with commensurate reduction in the size of the malignant lymphadenopathy. After 6 cycles of carboplatin and pemetrexed the patient proceeded directly to pemetrexed monotherapy. After 4 cycles of pemetrexed monotherapy, a repeat PET/CT scan showed no metabolic evidence of active disease (FIG. 13C, 13D). After 12 cycles of pemetrexed monotherapy, both CT and subsequent PET/CT showed isolated growth in two areas within the left hilar region, without re-emergence of activity elsewhere (FIG. 13E, 13F). The active areas were treated with stereotactic body radiation therapy. Following the radiation, therapy she restarted pemetrexed monotherapy. Subsequent CT scans demonstrated complete resolution of these foci. After 25 additional cycles of pemetrexed monotherapy, the patient developed 3 asymptomatic CNS metastases detected on surveillance MRI requiring stereotactic radiosurgery. Given that she had no evidence of systemic recurrence, the patient continued on pemetrexed monotherapy for 5 additional cycles, requiring stereotatic radiosurgery to 3 additional asymptomatic CNS metastases in October 2011. Again, there was no evidence of systemic recurrence on PET/CT, and the patient continued on pemetrexed monotherapy for 6 additional cycles when a 1.6 cm FDG avid right hilar lymph node was discovered on routine PET/CT surveillance. A fine needle aspirate was obtained that confirmed recurrent adenocarcinoma, but did not provide enough malignant cells for repeat molecular analysis. As this was her only site of progression, the patient elected to continue single agent pemetrexed after completion of 4000 cGy in 10 fractions to the area of disease. As of July 2012, she has received an additional 8 cycles of pemetrexed monotherapy with a total of 60 cycles of pemetrexed with no further evidence of recurrent or residual disease by either MRI or PET/CT. While the patient's progression free survival (PFS) on pemetrexed was 17 months, she has remained on pemetrexed therapy for a total of 46 months with intermittent oligometastatic disease that has been well controlled with radiotherapy. She continues to tolerate systemic chemotherapy without dose limiting toxicity or decline in her overall performance status.

### Case 2

A 45 year old male never smoker presented with persistent cough and noncardiac chest pain in June 2009. During the initial evaluation, a chest CT scan revealed a left upper lobe mass 7.6×5.9 cm in size with invasion of the left pulmonary hilum and associated subcarinal lymphadenopathy. CT of the abdomen and pelvis revealed a 4.8×2.1 cm lesion in the right iliac region. Pathology from transbronchial biopsy with pathology confirmed a moderately differentiated TTF-1 positive lung adenocarcinoma. Molecular testing revealed the patient's sample as having a G12V mutation in KRAS. Patient tissue was not available for FISH testing. PET/CT scan after initial consultation demonstrated the known primary, in addition to metastatic disease located in bilateral tracheal lymph nodes, right hilar lymph node, a left scalp lesion, and multiple foci including the left acromion, medial right clavicle, left superior pubic ramus, and the posterior right iliac bone (FIG. 13G, 13H). The patient began chemotherapy consisting of carboplatin, pemetrexed and zoledronic acid. Following 6 cycles, CT scan revealed stable disease with a 2.4×2.1 cm remaining left lung primary tumor. After 4 additional cycles of pemetrexed and zoledronic acid, PET/CT displayed no metabolically active disease (FIG. 13I, 13J) with the patient electing to continue pemetrexed monotherapy. Multiple subsequent staging CT examinations revealed a persistent 2.2×2.2 cm left lung primary and right hilar lymph node and another transbronchial biopsy was obtained. While the biopsy was nondiagnostic for evidence of tumor, the patient continued with an excellent performance status (ECOG 0) and elected to continue with additional cycles of pemetrexed monotherapy in absence of overt disease progression. In October 2011 the patient underwent stereotactic body radiation to the growing left lung primary. As this remained the only area of active progression, the patient elected to resume pemetrexed monotherapy upon completion of radiation. As of July 2012, there have been no additional areas of progression on surveillance CT and MRI. Six cycles of pemetrexed-platinum doublet therapy followed by 44 cycles of single agent pemetrexed maintenance has resulted in a progression free survival of 27 months in this patient (36 months of pemetrexed treatment in total).

### Example 9

### Predicting/Determining Responsiveness of a Mammalian Subject to an Antifolate

The success of therapeutics in medicine and especially in a complex disease such as cancer depends on the correct diagnosis choice of patients treated with a drug. This process requires knowledge of the specific patient markers that can be used to predict how the patient will respond to a given drug or class of drugs that share a common mechanism of action. The inventors of the instant application have shown that cells which harbor a RAS mutation and no amplification of Ras are responsive to an antifolate such as a DHFR inhibitor. A mammalian tumor likely to be responsive to a DHFR inhibitor may be identified as follows.

In an exemplary method, a biological sample was removed from subjects prior to treatment with an antifolate such as Methotrexate and analyzed for expression of one or more RAS mutations (e.g., one or more mutations in KRAS (SEQ ID NO: 1). The patient sample consisted of a tumor biopsy. The biological sample was then analyzed for the presence or absence of one or more KRAS mutations and KRAS amplification (e.g., three or more copies KRAS including seven or more copies of KRAS). Patient samples which exhibited a KRAS mutation (e.g., expression of mutated KRAS) and no amplification of KRAS were predicted to be responsive to treatment with the antifolate. Conversely, patient samples which did not exhibit a KRAS mutation (e.g., wild-type KRAS), irrespective of KRAS amplification, were predicted to not be responsive to treatment with antifolate. Patients predicted to be responsive to the antifolate, were then treated with the antifolate. After treatment of the patients with the antifolate, a biological sample (e.g., serum) was removed from the patients and tested for expression of one or more microRNAs (e.g., one or more miR-143 and miR-181 gene family members). Those patients which exhibited increased expression of such microRNAs were determined to be responding to treatment with the antifolate. Those patients that did not exhibit an increased expression of such microRNAs were determined to not be responding to treatment with the antifolate.

Aspects, including embodiments, of the present subject matter described above may be beneficial alone or in combination, with one or more other aspects or embodiments. Without limiting the foregoing description, in accordance with one aspect of the subject matter herein, there is provided a method for predicting and/or determining sensitivity of a test cell to a dihydrofolate reductase (DHFR) inhibitor, the method comprising: obtaining a test cell; contacting the test cell with a DHFR inhibitor; assaying the test cell for expression of one or more miRNAs; determining if expression of the one or more miRNAs in the test cell is elevated or reduced compared to a control cell or above or below a threshold; and employing the determination of the expression of the one or more miRNAs in the test cell to predict or determine sensitivity of the test cell to the DHFR inhibitor, wherein the test cell has mutated RAS and no amplification of the RAS gene.

In accordance with another aspect which may be used or combined with any of the preceding or following aspects, the miRNA is from the miR181 family (e.g., miR181c) and/or the miR143 family.

In accordance with another aspect which may be used or combined with any of the preceding or following aspects, the DHFR inhibitor is an antifolate such as Methotrexate or Pemetrexed.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the test cell is from a tumor biopsy.

In accordance with another aspect which may be used or combined with any of the preceding or following aspects, the test cell is from an aspirate, blood, or serum.

In accordance with another aspect which may be used or combined with any of the preceding or following aspects, the test cell is from a cancer patient.

In accordance with another aspect which may be used or combined with any of the preceding or following aspects, the test cell is predicted and/or determined to be sensitive to the DHFR inhibitor where expression of the one or more miRNAs in the test cell is elevated as compared to expression of the one or more miRNAs in the control cell or is above the threshold.

In accordance with another aspect which may be used or combined with any of the preceding or following aspects, the step of assaying the test cell for expression of one or more miRNAs is performed by in situ hybridization (ISH), Northern blot, qRT-PCR or microarray analysis.

In accordance with another aspect which may be used or combined with any of the preceding or following aspects, the threshold is set at a level of miRNA expression above which the control cell is known to be sensitive to treatment with the DHFR inhibitor and below which the control cell is known to not be sensitive to treatment with the DHFR inhibitor.

In accordance with another aspect which may be used or combined with any of the preceding or following aspects, the control cell is the same cell type as the test cell. In some embodiments, the control cell is a different cell type than the test cell.

In accordance with another aspect which may be used or combined with any of the preceding or following aspects, the threshold is set at a level of miRNA expression above which 50%, 60%, 70%, 80%, 90%, or 95% of control cells respond to treatment with the DHFR inhibitor.

In accordance with another aspect which may be used or combined with any of the preceding or following aspects, the threshold is set at a level of miRNA expression below which 50%, 60%, 70%, 80%, 90%, or 95% of control cells do not respond to treatment with the DHFR inhibitor.

Without limiting the foregoing description, in accordance with another aspect of the subject matter herein, there is provided a method for predicting and/or determining responsiveness of a subject with a disease or disorder to a dihydrofolate reductase (DHFR) inhibitor, the method comprising: administering a DHFR inhibitor to the subject; obtaining a biological sample from the subject; assaying the biological sample for expression of one or more miRNAs; determining if expression of the one or more miRNAs in the biological sample is elevated or reduced compared to a control sample or above or below a threshold; and employing the determination of the expression of the one or more miRNAs in the biological sample to predict and/or determine responsiveness of the subject to the DHFR inhibitor, wherein the biological sample comprises cells with mutated RAS and no amplification of the RAS gene.

In accordance with another aspect which may be used or combined with any of the preceding or following aspects, the miRNA is from the miR181 family (e.g., miR181c) and/or the miR143 family.

In accordance with another aspect which may be used or combined with any of the preceding or following aspects, the DHFR inhibitor is an antifolate such as Methotrexate or Pemetrexed.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the biological sample is from a tumor biopsy or blood sample.

In accordance with another aspect which may be used or combined with any of the preceding or following aspects, the biological sample is from an aspirate. In some embodiments, the biological sample is from a cancer patient.

In accordance with another aspect which may be used or combined with any of the preceding or following aspects, the subject is predicted and/or determined to be sensitive to the DHFR inhibitor where expression of the one or more miRNAs in the biological sample is elevated as compared to expression of the one or more miRNAs in the control sample or is above the threshold.

In accordance with another aspect which may be used or combined with any of the preceding or following aspects, the step of assaying the biological sample for expression of one or more miRNAs is performed by in situ hybridization (ISH), Northern blot, qRT-PCR or microarray analysis.

In accordance with another aspect which may be used or combined with any of the preceding or following aspects, the threshold is set at a level of miRNA expression in the control sample above which a subject is known to be sensitive to treatment with the DHFR inhibitor and below which a subject is known to not be sensitive to treatment with the DHFR inhibitor.

In accordance with another aspect which may be used or combined with any of the preceding or following aspects, the threshold is set at a level of miRNA expression above which 50%, 60%, 70%, 80%, 90%, or 95% of subject(s) respond to treatment with the DHFR inhibitor.

In accordance with another aspect which may be used or combined with any of the preceding or following aspects, the threshold is set at a level of miRNA expression below which 50%, 60%, 70%, 80%, 90%, or 95% of subject(s) do not respond to treatment with the DHFR inhibitor.

In accordance with another aspect which may be used or combined with any of the preceding or following aspects, the disease or disorder is cancer. In some embodiments, the cancer is selected from the group consisting of gastrointestinal, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

Without limiting the foregoing description, in accordance with another aspect of the subject matter herein, there is provided a method for treating a subject with a disease or disorder, the method comprising: administering a DHFR inhibitor to the subject; obtaining a biological sample from a subject; assaying the biological sample for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and administering to the subject a therapeutically effective amount of DHFR inhibitor where the subject is predicted and/or determined to be responsive to the DHFR inhibitor, wherein the biological sample comprises cells with mutated RAS and no amplification of the RAS gene.

In accordance with another aspect which may be used or combined with any of the preceding or following aspects, the miRNA is from the miR181 family (e.g., miR181c) and/or the miR143 family.

In accordance with another aspect which may be used or combined with any of the preceding or following aspects, the DHFR inhibitor is an antifolate such as Methotrexate or Pemetrexed.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the biological sample is from a tumor biopsy or blood sample.

In accordance with another aspect which may be used or combined with any of the preceding or following aspects, the biological sample is from an aspirate. In some embodiments, the biological sample is from a cancer patient.

In accordance with another aspect which may be used or combined with any of the preceding or following aspects, the subject is predicted and/or determined to be sensitive to the DHFR inhibitor where expression of the one or more miRNAs in the biological sample is elevated as compared to expression of the one or more miRNAs in the control sample or is above the threshold.

In accordance with another aspect which may be used or combined with any of the preceding or following aspects, the step of assaying the test cell for expression of one or more miRNAs is performed by in situ hybridization (ISH), Northern blot, qRT-PCR or microarray analysis.

In accordance with another aspect which may be used or combined with any of the preceding or following aspects, the threshold is set at a level of miRNA expression in a control sample above which a subject is known to be sensitive to treatment with the DHFR inhibitor and below which a subject is known to not be sensitive to treatment with the DHFR inhibitor.

In accordance with another aspect which may be used or combined with any of the preceding or following aspects, the threshold is set at a level of miRNA expression above which 50%, 60%, 70%, 80%, 90%, or 95% of subject(s) respond to treatment with the DHFR inhibitor.

In accordance with another aspect which may be used or combined with any of the preceding or following aspects, the threshold is set at a level of miRNA expression below which 50%, 60%, 70%, 80%, 90%, or 95% of subject(s) do not respond to treatment with the DHFR inhibitor.

In accordance with another aspect which may be used or combined with any of the preceding or following aspects, the disease or disorder is cancer.

In accordance with another aspect which may be used or combined with any of the preceding or following aspects, the cancer is selected from the group consisting of gastrointestinal, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the subject is a cancer patient.

In accordance with another aspect which may be used or combined with any of the preceding or following aspects, the therapeutically effective amount of one or more DHFR inhibitors are optionally adapted for a co-treatment with radiotherapy or radio-immunotherapy.

Without limiting the foregoing description, in accordance with another aspect of the subject matter herein, there is provided a method for selecting subjects for a clinical trial for testing the efficacy or safety of a dihydrofolate reductase (DHFR) inhibitor, the method comprising: administering a DHFR inhibitor to the subject; obtaining a biological sample from the subjects; assaying the biological samples obtained from the subjects for expression of one or more miRNAs; determining if expression of the one or more miRNAs in the biological sample is elevated or reduced compared to a control sample or above or below a threshold; employing the determination of the expression of the one or more miRNAs in the biological sample to predict and/or determine responsiveness of the subject to the DHFR inhibitor; and selecting subjects for inclusion in a clinical trial that are predicted and/or determined to be responsive to the DHFR inhibitor, wherein the biological sample comprises one or more cells with mutated RAS and no amplification of the RAS gene.

In accordance with another aspect which may be used or combined with any of the preceding or following aspects, the miRNA is from the miR181 family (e.g., miR181c) and/or the miR143 family.

In accordance with another aspect which may be used or combined with any of the preceding or following aspects, the DHFR inhibitor is an antifolate such as Methotrexate or Pemetrexed.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the biological sample is from a tumor biopsy or blood sample.

In accordance with another aspect which may be used or combined with any of the preceding or following aspects, the biological sample is from an aspirate.

In accordance with another aspect which may be used or combined with any of the preceding or following aspects, the biological sample is from a cancer patient.

In accordance with another aspect which may be used or combined with any of the preceding or following aspects, the subject is predicted and/or determined to be sensitive to the DHFR inhibitor where expression of the one or more miRNAs in the biological sample is elevated as compared to expression of the one or more miRNAs in the control sample or is above the threshold.

In accordance with another aspect which may be used or combined with any of the preceding or following aspects, the step of assaying the test cell for expression of one or more miRNAs is performed by in situ hybridization (ISH), Northern blot, qRT-PCR or microarray analysis.

In accordance with another aspect which may be used or combined with any of the preceding or following aspects, the threshold is set at a level of miRNA expression in a control sample above which a subject is known to be sensitive to treatment with the DHFR inhibitor and below which a subject is known to not be sensitive to treatment with the DHFR inhibitor.

In accordance with another aspect which may be used or combined with any of the preceding or following aspects, the threshold is set at a level of miRNA expression above which 50%, 60%, 70%, 80%, 90%, or 95% of subject(s) respond to treatment with the DHFR inhibitor.

In accordance with another aspect which may be used or combined with any of the preceding or following aspects, the threshold is set at a level of miRNA expression below which 50%, 60%, 70%, 80%, 90%, or 95% of subject(s) do not respond to treatment with the DHFR inhibitor.

In accordance with another aspect which may be used or combined with any of the preceding or following aspects, the disease or disorder is cancer. In some embodiments, the cancer is selected from the group consisting of gastrointestinal, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the subject is a cancer patient.

In accordance with another aspect which may be used or combined with any of the preceding or following aspects, the method may further comprise seeking regulatory approval for the DHFR inhibitor.

In accordance with another aspect which may be used or combined with any of the preceding or following aspects, the clinical trial is a phase I, phase II, phase III or phase IV clinical trial.

Without limiting the foregoing description, in accordance with another aspect of the subject matter herein, there is provided a method for predicting sensitivity of a test cell to a DHFR inhibitor, by obtaining a test cell; assaying the test cell for one or more RAS mutations; determining if one or more RAS mutations are present or absent in the test cell; and employing the determination of the presence or absence of a RAS mutation in the test cell to predict sensitivity of the test cell to the drug.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the DHFR inhibitor is Methotrexate or Pemetrexed.

In accordance with another aspect which may be used or combined with any of the preceding or following aspects, the test cell is obtained from a subject that has a disease or disorder.

In accordance with another aspect which may be used or combined with any of the preceding or following aspects, the disease or disorder is cancer.

In accordance with another aspect which may be used or combined with any of the preceding or following aspects, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the subject is a cancer patient.

In accordance with another aspect which may be used or combined with any of the preceding or following aspects, the test cell is assayed for one or more RAS mutations by analyzing nucleic acid obtained from the test cell.

In accordance with another aspect which may be used or combined with any of the preceding or following aspects, the test cell is assayed for one or more RAS mutations by analyzing proteins obtained from the test cell.

In accordance with another aspect which may be used or combined with any of the preceding or following aspects, test cell is obtained from a tumor biopsy.

In accordance with another aspect which may be used or combined with any of the preceding or following aspects, the test cell is obtained from an aspirate, blood or serum.

In accordance with another aspect which may be used or combined with any of the preceding or following aspects, the test cell is predicted to be sensitive to the DHFR inhibitor where one or more RAS mutations are determined to be present in the test cell.

In accordance with another aspect which may be used or combined with any of the preceding or following aspects, the test cell is predicted to be sensitive to the DHFR inhibitor where RAS mutations are determined to be absent in the test cell.

In accordance with another aspect which may be used or combined with any of the preceding or following aspects, the test cell is predicted to be insensitive to the DHFR inhibitor where one or more RAS mutations are determined to be present in the test cell.

In accordance with another aspect which may be used or combined with any of the preceding or following aspects, the test cell is predicted to be insensitive to the DHFR inhibitor where one or more RAS mutations are determined to be absent in the test cell.

In accordance with another aspect which may be used or combined with any of the preceding or following aspects, the step of assaying the test cell for one or more RAS mutations is performed by in situ hybridization (ISH), Northern blot, qRT-PCR or microarray analysis.

Without limiting the foregoing description, in accordance with another aspect of the subject matter herein, there is provided a method for selecting subjects for inclusion in a clinical trial for testing the efficacy or safety of a DHFR inhibitor by obtaining a biological sample comprising target cells from the subject; assaying target cells in the biological sample for one or more RAS mutations; determining if one or more RAS mutations are present or absent in the target cells; employing the determination of the presence or absence of a RAS mutation in the target cells to predict sensitivity of the target cells to the DHFR inhibitor; and selecting subjects for inclusion in the clinical that are predicted to be responsive to the DHFR inhibitor.

In accordance with another aspect which may be used or combined with any of the preceding or following aspects, subjects are selected for the clinical trial that have one or more RAS mutations present in target cells from their biological sample.

In accordance with another aspect which may be used or combined with any of the preceding or following aspects, subjects are selected for the clinical trial that have one or more RAS mutations absent in target cells from their biological sample.

Without limiting the foregoing description, in accordance with another aspect of the subject matter herein, there is provided a method for predicting responsiveness of a subject with a disease or disorder to treatment with a DHFR inhibitor by obtaining a biological sample from the subject; assaying target cells obtained from the biological sample for one or more RAS mutations; determining if one or more RAS mutations are present or absent in the target cells; and employing the determination of the presence or absence of a RAS mutation in the target cells obtained from the biological sample to predict responsiveness of the subject to the DHFR inhibitor.

In accordance with another aspect which may be used or combined with any of the preceding or following aspects, the subject is predicted to be responsive to the DHFR inhibitor where one or more RAS mutations are present in the target cells.

In accordance with another aspect which may be used or combined with any of the preceding or following aspects, the subject is predicted to be responsive to the DHFR inhibitor where one or more RAS mutations are absent in the target cells.

In accordance with another aspect which may be used or combined with any of the preceding or following aspects, the subject is predicted to be non-responsive to the DHFR inhibitor where one or more RAS mutations are present in the target cells.

In accordance with another aspect which may be used or combined with any of the preceding or following aspects, the subject is predicted to be non-responsive to the DHFR inhibitor where one or more RAS mutations are absent in the target.

Without limiting the foregoing description, in accordance with another aspect of the subject matter herein, there is provided a method for treating a subject with a disease or disorder by obtaining a biological sample from a subject; assaying target cells obtained from the biological sample for one or more RAS mutations; determining if one or more RAS mutations are present or absent in the target cells; employing the determination of the presence or absence of a RAS mutation in the target cells obtained from the biological sample to predict responsiveness of the subject to a DHFR inhibitor; and administering to the subject a therapeutically effective amount of the DHFR inhibitor where the subject is predicted to be responsive to the DHFR inhibitor.

In accordance with another aspect which may be used or combined with any of the preceding or following aspects, the subject is predicted to be responsive to the DHFR inhibitor where one or more RAS mutations are present in the target cells.

In accordance with another aspect which may be used or combined with any of the preceding or following aspects, the subject is predicted to be responsive to the drug where one or more RAS mutations are absent in the target cells.

Without limiting the foregoing description, in accordance with another aspect of the subject matter herein, there is provided a method for predicting sensitivity of a test cell to an DHFR inhibitor by obtaining a test cell; assaying the test cell for one or more RAS mutations; determining if the test cell has one or more RAS mutations; and employing the determination of the presence of absence of a RAS mutation in the test cell to predict sensitivity of the test cell to the DHFR inhibitor, wherein the presence of a RAS mutation predicts that the test cell will be sensitive to the DHFR inhibitor, the absence of a RAS mutation predicts that the test cell will be sensitive to the DHFR inhibitor, the presence of a RAS mutation predicts that the test cell will be insensitive to the DHFR inhibitor, or the absence of a RAS mutation predicts that the test cell will be insensitive to the DHFR inhibitor.

Without limiting the foregoing description, in accordance with another aspect of the subject matter herein, there is provided a method for predicting sensitivity of a test cell to a drug by obtaining a test cell; assaying the test cell for one or more RAS mutations; assaying the test cell for amplification of a RAS gene; determining if one or more RAS mutations are present or absent in the test cell and determining if an amplification of the RAS gene is present or absent in the test cell; and employing the determination of the presence or absence of a RAS mutation in the test cell and the presence or absence of an amplification of RAS in the test cell to predict sensitivity of the test cell to the drug.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the RAS amplification is one or more of an amplification of KRAS (SEQ ID NO: 4), NRAS (SEQ ID NO: 5) or HRAS (SEQ ID NO: 6).

In accordance with another aspect which may be used or combined with any of the preceding or following aspects, the drug is a chemotherapeutic agent.

In accordance with another aspect which may be used or combined with any of the preceding or following aspects, the drug is an EGFR targeted therapy.

In accordance with another aspect which may be used or combined with any of the preceding or following aspects, the drug is an antifolate such as a dihydrofolate reductase (DHFR) inhibitor.

In accordance with another aspect which may be used or combined with any of the preceding or following aspects, the DHFR inhibitor is Methotrexate or Pemetrexed.

In accordance with another aspect which may be used or combined with any of the preceding or following aspects, the EGFR targeted therapy is a tyrosine kinase inhibitor that targets HER1 (EGFR), HER2/neu, HER3, or any combination thereof.

In accordance with another aspect which may be used or combined with any of the preceding or following aspects, the tyrosine kinase inhibitor is an antibody.

In accordance with another aspect which may be used or combined with any of the preceding or following aspects, the antibody is a monoclonal antibody.

In accordance with another aspect which may be used or combined with any of the preceding or following aspects, the monoclonal antibody is cetuximab (Erbitux), panitumumab, zalutumumab, nimotuzumab or matuzumab. In some embodiments, the tyrosine kinase inhibitor is a small molecule inhibitor. In some embodiments, the small molecule inhibitor is gefitinib, erlotinib or lapatinib.

In accordance with another aspect which may be used or combined with any of the preceding or following aspects, the test cell is obtained from a subject that has a disease or disorder.

In accordance with another aspect which may be used or combined with any of the preceding or following aspects, the subject is a cancer patient.

In accordance with another aspect which may be used or combined with any of the preceding or following aspects, the disease or disorder is cancer.

In accordance with another aspect which may be used or combined with any of the preceding or following aspects, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the test cell is assayed for one or more RAS mutations and an amplification of RAS by analyzing nucleic acid obtained from the test cell. In accordance with another aspect which may be used or combined with any of the preceding or following aspects, the test cell is assayed for one or more RAS mutations by analyzing proteins obtained from the test cell.

In accordance with another aspect which may be used or combined with any of the preceding or following aspects, the test cell is obtained from a tumor biopsy.

In accordance with another aspect which may be used or combined with any of the preceding or following aspects, the test cell is obtained from an aspirate, blood or serum.

In accordance with another aspect which may be used or combined with any of the preceding or following aspects, the test cell is predicted to be sensitive to the drug where one or more RAS mutations are determined to be present in the test cell and an amplification of RAS is determined to be present in the test cell.

In accordance with another aspect which may be used or combined with any of the preceding or following aspects, the test cell is predicted to be sensitive to the drug where one or more RAS mutations are determined to be present in the test cell and amplification of RAS is determined to be absent in the test cell.

In accordance with another aspect which may be used or combined with any of the preceding or following aspects, the test cell is predicted to be sensitive to the drug where RAS mutations are determined to be absent in the test cell and an amplification of RAS is determined to be present in the test cell.

In accordance with another aspect which may be used or combined with any of the preceding or following aspects, the test cell is predicted to be sensitive to the drug where RAS mutations are determined to be absent in the test cell and amplification of RAS is determined to be absent in the test cell.

In some embodiments, In accordance with another aspect which may be used or combined with any of the preceding or following aspects, the test cell is predicted to be insensitive to the drug where one or more RAS mutations are determined to be present in the test cell and an amplification of RAS is determined to be present in the test cell.

In accordance with another aspect which may be used or combined with any of the preceding or following aspects, the test cell is predicted to be insensitive to the drug where one or more RAS mutations are determined to be present in the test cell and amplification of RAS is determined to be absent in the test cell.

In accordance with another aspect which may be used or combined with any of the preceding or following aspects, the test cell is predicted to be insensitive to the drug where RAS mutations are determined to be absent in the test cell and an amplification of RAS is determined to be present in the test cell.

In accordance with another aspect which may be used or combined with any of the preceding or following aspects, the test cell is predicted to be insensitive to the drug where RAS mutations are determined to be absent in the test cell and amplification of RAS is determined to be absent in the test cell.

In accordance with another aspect which may be used or combined with any of the preceding or following aspects, the step of assaying the test cell for one or more RAS mutations and amplification of RAS is performed by in situ hybridization (ISH), Northern blot, qRT-PCR or microarray analysis.

Without limiting the foregoing description, in accordance with another aspect of the subject matter herein, there is provided a method for selecting subjects for inclusion in a clinical trial including, a clinical trial for testing the efficacy or safety of a drug, by obtaining a biological sample comprising target cells from the subject; assaying target cells in the biological sample for one or more RAS mutations; assaying target cells in the biological sample for an amplification RAS; determining if one or more RAS mutations are present or absent in the target cells and determining if an amplification of the RAS gene is present or absent in the target cells; employing the determination of the presence or absence of a RAS mutation in the target cells and the presence or absence of an amplification of RAS in the target cells to predict sensitivity of the target cells to the drug; and selecting subjects for inclusion in the clinical that are predicted to be responsive to the drug.

Without limiting the foregoing description, in accordance with another aspect of the subject matter herein, there is provided a method for selecting subjects for inclusion in a clinical trial for testing the efficacy or safety of a drug by obtaining a biological sample comprising target cells from the subject; determining if the cells have one or more RAS mutations; determining if the cells have a RAS amplification; and selecting subjects for inclusion in the clinical with the drug based upon the determination of whether the target cells have one or more RAS mutations and a RAS amplification.

In accordance with another aspect which may be used or combined with any of the preceding or following aspects, the subjects that have one or more RAS mutations and a RAS amplification are selected for inclusion in the clinical trial. In another embodiment, the subjects that have one or more RAS mutations and do not have a RAS amplification are selected for inclusion in the clinical trial. In yet another embodiment, the subjects that do not have one or more RAS mutations and have a RAS amplification are selected for inclusion in the clinical trial. In another embodiment, the subjects that do not have one or more RAS mutations and do not have a RAS amplification are selected for inclusion in the clinical trial.

In accordance with another aspect which may be used or combined with any of the preceding or following aspects, subjects are selected for the clinical trial that have one or more RAS mutations present in target cells from their biological sample and that have an amplification of RAS present in target cells from their biological sample.

In accordance with another aspect which may be used or combined with any of the preceding or following aspects, subjects are selected for the clinical trial that have one or more RAS mutations absent in target cells from their biological sample and that have an amplification of RAS present in target cells from their biological sample.

In accordance with another aspect which may be used or combined with any of the preceding or following aspects, subjects are selected for the clinical trial that have one or more RAS mutations present in target cells from their biological sample and that have an amplification of RAS absent in target cells from their biological sample.

In accordance with another aspect which may be used or combined with any of the preceding or following aspects, subjects are selected for the clinical trial that have one or more RAS mutations absent in target cells from their biological sample and that have an amplification of RAS absent in target cells from their biological sample.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the RAS amplification is one or more of an amplification of KRAS (SEQ ID NO: 4), NRAS (SEQ ID NO: 5) or HRAS (SEQ ID NO: 6).

In accordance with another aspect which may be used or combined with any of the preceding or following aspects, the drug is a chemotherapeutic agent.

In accordance with another aspect which may be used or combined with any of the preceding or following aspects, the drug is an EGFR targeted therapy.

In accordance with another aspect which may be used or combined with any of the preceding or following aspects, the drug is an antifolate such as a dihydrofolate reductase (DHFR) inhibitor.

In accordance with another aspect which may be used or combined with any of the preceding or following aspects, the DHFR inhibitor is Methotrexate or Pemetrexed.

In accordance with another aspect which may be used or combined with any of the preceding or following aspects, the EGFR targeted therapy is a tyrosine kinase inhibitor that targets HER1 (EGFR), HER2/neu, HER3, or any combination thereof.

In accordance with another aspect which may be used or combined with any of the preceding or following aspects, the tyrosine kinase inhibitor is an antibody.

In accordance with another aspect which may be used or combined with any of the preceding or following aspects, the antibody is a monoclonal antibody.

In accordance with another aspect which may be used or combined with any of the preceding or following aspects, the monoclonal antibody is cetuximab (Erbitux), panitumumab, zalutumumab, nimotuzumab or matuzumab.

In accordance with another aspect which may be used or combined with any of the preceding or following aspects, the tyrosine kinase inhibitor is a small molecule inhibitor.

In accordance with another aspect which may be used or combined with any of the preceding or following aspects, the small molecule inhibitor is gefitinib, erlotinib or lapatinib.

In accordance with another aspect which may be used or combined with any of the preceding or following aspects, the biological sample is obtained from a subject that has a disease or disorder.

In accordance with another aspect which may be used or combined with any of the preceding or following aspects, the subject is a cancer patient.

In accordance with another aspect which may be used or combined with any of the preceding or following aspects, the disease or disorder is cancer.

In accordance with another aspect which may be used or combined with any of the preceding or following aspects, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the biological sample (e.g., one or more cells in the biological sample) is assayed for one or more RAS mutations and an amplification of RAS by analyzing nucleic acid obtained from the test cell.

In accordance with another aspect which may be used or combined with any of the preceding or following aspects, the biological sample (e.g., one or more cells in the biological sample) is assayed for one or more RAS mutations by analyzing proteins obtained from the test cell.

In accordance with another aspect which may be used or combined with any of the preceding or following aspects, the biological sample is obtained from a tumor biopsy.

In accordance with another aspect which may be used or combined with any of the preceding or following aspects, the biological sample is obtained from an aspirate, blood or serum.

In accordance with another aspect which may be used or combined with any of the preceding or following aspects, the step of assaying target cells in the biological sample for one or more RAS mutations and amplification of RAS is performed by in situ hybridization (ISH), Northern blot, qRT-PCR or microarray analysis.

Without limiting the foregoing description, in accordance with another aspect of the subject matter herein, there is provided a method for predicting responsiveness of a subject with a disease or disorder to treatment with a drug by obtaining a biological sample from the subject; assaying target cells obtained from the biological sample for one or more RAS mutations; assaying target cells obtained from the biological sample for a RAS amplification; determining if one or more RAS mutations are present or absent in the target cells and determining if an amplification of the RAS gene is present or absent in the target cells; and employing the determination of the presence or absence of a RAS mutation and the presence or absence of an amplification of RAS in the target cells obtained from the biological sample to predict responsiveness of the subject to the drug.

In accordance with another aspect which may be used or combined with any of the preceding or following aspects, the subject is predicted to be responsive to the drug where one or more RAS mutations are present in the target cells and an amplification of RAS is present in the target cells.

In accordance with another aspect which may be used or combined with any of the preceding or following aspects, the subject is predicted to be responsive to the drug where one or more RAS mutations are absent in the target cells and an amplification of RAS is present in the target cells.

In accordance with another aspect which may be used or combined with any of the preceding or following aspects, the subject is predicted to be responsive to the drug where one or more RAS mutations are present in the target cells and an amplification of RAS is absent in the target cells.

In accordance with another aspect which may be used or combined with any of the preceding or following aspects, the subject is predicted to be responsive to the drug where one or more RAS mutations are absent in the target cells and an amplification of RAS is absent in the target cells.

In accordance with another aspect which may be used or combined with any of the preceding or following aspects, the subject is predicted to be non-responsive to the drug where one or more RAS mutations are present in the target cells and an amplification of RAS is present in the target cells.

In accordance with another aspect which may be used or combined with any of the preceding or following aspects, the subject is predicted to be non-responsive to the drug where one or more RAS mutations are absent in the target cells and an amplification of RAS is present in the target cells.

In accordance with another aspect which may be used or combined with any of the preceding or following aspects, the subject is predicted to be non-responsive to the drug where one or more RAS mutations are present in the target cells and an amplification of RAS is absent in the target cells.

In accordance with another aspect which may be used or combined with any of the preceding or following aspects, the subject is predicted to be non-responsive to the drug where one or more RAS mutations are absent in the target cells and an amplification of RAS is absent in the target cells.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the RAS amplification is one or more of an amplification of KRAS (SEQ ID NO: 4), NRAS (SEQ ID NO: 5) or HRAS (SEQ ID NO: 6).

In accordance with another aspect which may be used or combined with any of the preceding or following aspects, the drug is a chemotherapeutic agent.

In accordance with another aspect which may be used or combined with any of the preceding or following aspects, the drug is an EGFR targeted therapy.

In accordance with another aspect which may be used or combined with any of the preceding or following aspects, the drug is an antifolate such as a dihydrofolate reductase (DHFR) inhibitor.

In accordance with another aspect which may be used or combined with any of the preceding or following aspects, the DHFR inhibitor is Methotrexate or Pemetrexed.

In accordance with another aspect which may be used or combined with any of the preceding or following aspects, the EGFR targeted therapy is a tyrosine kinase inhibitor that targets HER1 (EGFR), HER2/neu, HER3, or any combination thereof.

In accordance with another aspect which may be used or combined with any of the preceding or following aspects, the tyrosine kinase inhibitor is an antibody.

In accordance with another aspect which may be used or combined with any of the preceding or following aspects, the antibody is a monoclonal antibody.

In accordance with another aspect which may be used or combined with any of the preceding or following aspects, the monoclonal antibody is cetuximab (Erbitux), panitumumab, zalutumumab, nimotuzumab or matuzumab.

In accordance with another aspect which may be used or combined with any of the preceding or following aspects, the tyrosine kinase inhibitor is a small molecule inhibitor.

In accordance with another aspect which may be used or combined with any of the preceding or following aspects, the small molecule inhibitor is gefitinib, erlotinib or lapatinib.

In accordance with another aspect which may be used or combined with any of the preceding or following aspects, the biological sample is obtained from a subject that has a disease or disorder.

In accordance with another aspect which may be used or combined with any of the preceding or following aspects, the subject is a cancer patient.

In accordance with another aspect which may be used or combined with any of the preceding or following aspects, the disease or disorder is cancer.

In accordance with another aspect which may be used or combined with any of the preceding or following aspects, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the biological sample (e.g., one or more cells in the biological sample) is assayed for one or more RAS mutations and an amplification of RAS by analyzing nucleic acid obtained from the test cell.

In accordance with another aspect which may be used or combined with any of the preceding or following aspects, the biological sample (e.g., one or more cells in the biological sample) is assayed for one or more RAS mutations by analyzing proteins obtained from the test cell.

In accordance with another aspect which may be used or combined with any of the preceding or following aspects, the biological sample is obtained from a tumor biopsy.

In accordance with another aspect which may be used or combined with any of the preceding or following aspects, the biological sample is obtained from an aspirate, blood or serum.

In accordance with another aspect which may be used or combined with any of the preceding or following aspects, the step of assaying target cells in the biological sample for one or more RAS mutations and amplification of RAS is performed by in situ hybridization (ISH), Northern blot, qRT-PCR or microarray analysis.

Without limiting the foregoing description, in accordance with another aspect of the subject matter herein, there is provided a method for treating a subject with a disease or disorder by obtaining a biological sample from a subject; assaying target cells obtained from the biological sample for one or more RAS mutations; assaying target cells obtained from the biological sample for a RAS amplification; determining if one or more RAS mutations are present or absent in the target cells and determining if an amplification of the RAS gene is present or absent in the target cells; employing the determination of the presence or absence of a RAS mutation and the presence or absence of an amplification of RAS in the target cells obtained from the biological sample to predict responsiveness of the subject to a drug; and administering to the subject a therapeutically effective amount of the drug where the subject is predicted to be responsive to the drug.

In accordance with another aspect which may be used or combined with any of the preceding or following aspects, the subject is predicted to be responsive to the drug where one or more RAS mutations are present in the target cells and an amplification of RAS is present in the target cells.

In accordance with another aspect which may be used or combined with any of the preceding or following aspects, the subject is predicted to be responsive to the drug where one or more RAS mutations are absent in the target cells and an amplification of RAS is present in the target cells.

In accordance with another aspect which may be used or combined with any of the preceding or following aspects, the subject is predicted to be responsive to the drug where one or more RAS mutations are present in the target cells and an amplification of RAS is absent in the target cells.

In accordance with another aspect which may be used or combined with any of the preceding or following aspects, the subject is predicted to be responsive to the drug where one or more RAS mutations are absent in the target cells and an amplification of RAS is absent in the target cells.

In accordance with another aspect which may be used or combined with any of the preceding or following aspects, RAS is KRAS (SEQ ID NO: 1), NRAS (SEQ ID NO: 2) or HRAS (SEQ ID NO: 3).

In accordance with another aspect which may be used or combined with any of the preceding or following aspects, the KRAS mutations are at one or more of positions 12, 13 or 61.

In accordance with another aspect which may be used or combined with any of the preceding or following aspects, the KRAS mutations are selected from the group consisting of: G12A, G12N, G12R, G12C, G12S, G12V, G13N and Q61H.

In accordance with another aspect which may be used or combined with any of the preceding or following aspects, the HRAS or NRAS mutations are at one or more of positions 12, 13 or 61.

In accordance with another aspect which may be used or combined with any of the preceding or following aspects, the RAS amplification is one or more of an amplification of KRAS (SEQ ID NO: 4), NRAS (SEQ ID NO: 5) or HRAS (SEQ ID NO: 6).

In accordance with another aspect which may be used or combined with any of the preceding or following aspects, the drug is a chemotherapeutic agent.

In accordance with another aspect which may be used or combined with any of the preceding or following aspects, the drug is an EGFR targeted therapy.

In accordance with another aspect which may be used or combined with any of the preceding or following aspects, the drug is an antifolate such as a dihydrofolate reductase (DHFR) inhibitor.

In accordance with another aspect which may be used or combined with any of the preceding or following aspects, the DHFR inhibitor is Methotrexate or Pemetrexed.

In accordance with another aspect which may be used or combined with any of the preceding or following aspects, the EGFR targeted therapy is a tyrosine kinase inhibitor that targets HER1 (EGFR), HER2/neu, HER3, or any combination thereof.

In accordance with another aspect which may be used or combined with any of the preceding or following aspects, the tyrosine kinase inhibitor is an antibody.

In accordance with another aspect which may be used or combined with any of the preceding or following aspects, the antibody is a monoclonal antibody.

In accordance with another aspect which may be used or combined with any of the preceding or following aspects, the monoclonal antibody is cetuximab (Erbitux), panitumumab, zalutumumab, nimotuzumab or matuzumab.

In accordance with another aspect which may be used or combined with any of the preceding or following aspects, the tyrosine kinase inhibitor is a small molecule inhibitor.

In accordance with another aspect which may be used or combined with any of the preceding or following aspects, the small molecule inhibitor is gefitinib, erlotinib or lapatinib.

In accordance with another aspect which may be used or combined with any of the preceding or following aspects, the biological sample is obtained from a subject that has a disease or disorder.

In accordance with another aspect which may be used or combined with any of the preceding or following aspects, the subject is a cancer patient.

In accordance with another aspect which may be used or combined with any of the preceding or following aspects, the disease or disorder is cancer.

In accordance with another aspect which may be used or combined with any of the preceding or following aspects, the cancer is selected from the group consisting of gastrointestinal cancer, prostate cancer, ovarian cancer, breast cancer, head and neck cancer, lung cancer, non-small cell lung cancer, cancer of the nervous system, kidney cancer, retina cancer, skin cancer, liver cancer, pancreatic cancer, genital-urinary cancer and bladder cancer.

In accordance with another aspect which may be used or combined with any of the preceding or following aspects, the biological sample (e.g., one or more cells in the biological sample) is assayed for one or more RAS mutations and an amplification of RAS by analyzing nucleic acid obtained from the test cell.

In accordance with another aspect which may be used or combined with any of the preceding or following aspects, the biological sample (e.g., one or more cells in the biological sample) is assayed for one or more RAS mutations by analyzing proteins obtained from the test cell.

In accordance with another aspect which may be used or combined with any of the preceding or following aspects, the biological sample is obtained from a tumor biopsy.

In accordance with another aspect which may be used or combined with any of the preceding or following aspects, the biological sample is obtained from an aspirate, blood or serum.

In accordance with another aspect which may be used or combined with any of the preceding or following aspects, the step of assaying target cells in the biological sample for one or more RAS mutations and amplification of RAS is performed by in situ hybridization (ISH), Northern blot, qRT-PCR or microarray analysis.

Without limiting the foregoing description, in accordance with another aspect of the subject matter herein, there is provided a method for predicting sensitivity of a test cell to a DHFR inhibitor by obtaining a test cell; assaying the test cell for one or more RAS mutations; assaying the test cell for amplification of a RAS gene; determining if the test cell has one or more RAS mutations and an amplification of the RAS gene; and employing the determination of the presence of absence of a RAS mutation and amplification of RAS in the test cell to predict sensitivity of the test cell to the drug, wherein the presence of a RAS mutation and the presence of an amplification of RAS predicts that the test cell will be insensitive to the DHFR inhibitor, the presence of a RAS mutation and the absence of a RAS amplification predicts that the test cell will be insensitive to the DHFR inhibitor, the absence of a RAS mutation and the presence of an amplification of RAS predicts that the test cell will be sensitive to the DHFR inhibitor or the absence of a RAS mutation and the absence of an amplification of RAS predicts that the test cell will be insensitive to the DHFR inhibitor.

While the present disclosure has been described and illustrated herein by references to various specific materials, procedures and examples, it is understood that the disclosure is not restricted to the particular combinations of materials and procedures selected for that purpose. Numerous variations of such details can be implied as will be appreciated by those skilled in the art. It is intended that the specification and examples be considered as exemplary, only, with the true scope and spirit of the disclosure being indicated by the following claims. All references, patents, and patent applications referred to in this application are herein incorporated by reference in their entirety.

1. A method for determining sensitivity of a test cell to a dihydrofolate reductase (DHFR) inhibitor, the method comprising:
a. contacting a test cell with a DHFR inhibitor;
b. assaying the test cell for expression of one or more miRNAs;
c. determining if expression of the one or more miRNAs in the test cell is elevated or reduced compared to a control cell or above or below a threshold; and
d. employing the determination of the expression of the one or more miRNAs in the test cell to determine sensitivity of the test cell to the DHFR inhibitor,
wherein the test cell has mutated RAS and no amplification of the RAS gene.

2. The method of claim 1, wherein the miRNA is from the miR-181 family and/or the miR143 family.

3. The method of claim 1, wherein the DHFR inhibitor is an antifolate.

4. The method of claim 3, wherein the antifolate is Methotrexate or Pemetrexed.

5. The method of claim 4, wherein RAS is KRAS (SEQ ID NO: 1).

6. The method of claim 5, wherein the KRAS mutations are at one or more of positions 12, 13 or 61.

7. The method of claim 5, wherein the KRAS mutations are selected from the group consisting of: G12A, G12N, G12R, G12C, G12S, G12V, G13N and Q61H.

8. The method of claim 1, wherein the test cell is from a tumor biopsy.

9. The method of claim 1, wherein the test cell is from an aspirate, blood, or serum.

10. The method of claim 1, wherein the test cell is from a cancer patient.

11. The method of claim 1, wherein the test cell is determined to be sensitive to the DHFR inhibitor where expression of the one or more miRNAs in the test cell is elevated as compared to expression of the one or more miRNAs in the control cell or is above the threshold.

12. The method of claim 1, wherein the step of assaying the test cell for expression of one or more miRNAs is performed by in situ hybridization (ISH), Northern blot, qRT-PCR or microarray analysis.

13. The method of claim 1, wherein the threshold is set at a level of miRNA expression above which the control cell is known to be sensitive to treatment with the DHFR inhibitor and below which the control cell is known to not be sensitive to treatment with the DHFR inhibitor.

14. The method of claim 11, wherein the control cell is the same cell type as the test cell.

15. The method of claim 11, wherein the control cell is a different cell type than the test cell.

16. The method of claim 11, wherein the threshold is set at a level of miRNA expression above which 50%, 60%, 70%, 80%, 90%, or 95% of control cells respond to treatment with the DHFR inhibitor.

17. The method of claim 11, wherein the threshold is set at a level of miRNA expression below which 50%, 60%, 70%, 80%, 90%, or 95% of control cells do not respond to treatment with the DHFR inhibitor.

18. A method for screening one or more dihydrofolate reductase (DHFR) inhibitors for a pharmacological activity, the method comprising:
a. contacting a test cell with the one or more DHFR inhibitors;
b. assaying the contacted test cell for expression of one or more miRNAs;
c. determining if expression of the one or more miRNAs in the test cell is elevated or reduced compared to a control cell or above or below a threshold; and
d. employing the determination of the expression of the one or more miRNAs in the test cell to determine a pharmacological activity of the DHFR inhibitor,
wherein the test cell has mutated RAS and no amplification of the RAS gene.

19-35. (canceled)

36. A method for determining responsiveness of a subject with a disease or disorder to a dihydrofolate reductase (DHFR) inhibitor, the method comprising:
a. administering a DHFR inhibitor to the subject;
b. obtaining a biological sample from the subject;
c. assaying the biological sample for expression of one or more miRNAs;
d. determining if expression of the one or more miRNAs in the biological sample is elevated or reduced compared to a control sample or above or below a threshold; and
e. employing the determination of the expression of the one or more miRNAs in the biological sample to determine responsiveness of the subject to the DHFR inhibitor,
wherein the biological sample comprises cells with mutated RAS and no amplification of the RAS gene.

37-56. (canceled)
